SEQUENCE LISTING (121 sequences)

SEQ ID NO: 1 - DNA, 23 bp, synthetic construct
  source 1..23: mol_type = other DNA
cgtttttttgtttttcgagtgcg

SEQ ID NO: 2 - DNA, 23 bp, synthetic construct
  source 1..23: mol_type = other DNA
tcaataactaaactcaccgcgtc

SEQ ID NO: 3 - DNA, 33 bp, synthetic construct
  source 1..33: mol_type = other DNA
aggccacggacggcggatttatcgggttatagt

SEQ ID NO: 4 - DNA, 32 bp, synthetic construct
  source 1..32: mol_type = other DNA
agccggttttccggctgagacgtccgtggcct

SEQ ID NO: 5 - DNA, 17 bp, synthetic construct
  source 1..17: mol_type = other DNA
gcggtaggagggttcgg

SEQ ID NO: 6 - DNA, 23 bp, synthetic construct
  source 1..23: mol_type = other DNA
ctcgacctcactcctattatcgc

SEQ ID NO: 7 - DNA, 25 bp, synthetic construct
  source 1..25: mol_type = other DNA
cgcgccgagggcgggagtttacgcg

SEQ ID NO: 8 - DNA, 30 bp, synthetic construct
  source 1..30: mol_type = other DNA
agccggttttccggctgagacctcggcgcg

SEQ ID NO: 9 - DNA, 28 bp, synthetic construct
  source 1..28: mol_type = other DNA
ggtttattttggttttttgagttttcgg

SEQ ID NO: 10 - DNA, 24 bp, synthetic construct
  source 1..24: mol_type = other DNA
tccaacctactatatttacgcgaa

SEQ ID NO: 11 - DNA, 35 bp, synthetic construct
  source 1..35: mol_type = other DNA
acggacgcggaggcggatttagggtatttaaggag

SEQ ID NO: 12 - DNA, 32 bp, synthetic construct; identical to SEQ ID NO: 4
  source 1..32: mol_type = other DNA

SEQ ID NO: 13 - DNA, 21 bp, synthetic construct
  source 1..21: mol_type = other DNA
gtatagcgcgatttcgtagcg

SEQ ID NO: 14 - DNA, 21 bp, synthetic construct
  source 1..21: mol_type = other DNA
aattacctacgctatccgccc

SEQ ID NO: 15 - DNA, 31 bp, synthetic construct
  source 1..31: mol_type = other DNA
aggccacggacgcgaacatcctcccgataat

SEQ ID NO: 16 - DNA, 18 bp, synthetic construct
  source 1..18: mol_type = other DNA
gttgtcgggagcggtagg

SEQ ID NO: 17 - DNA, 21 bp, synthetic construct
  source 1..21: mol_type = other DNA
ccaatatcccgaaacgcgtct

SEQ ID NO: 18 - DNA, 24 bp, synthetic construct
  source 1..24: mol_type = other DNA
cgcgccgagggcgtttcgatcggg

SEQ ID NO: 19 - DNA, 19 bp, synthetic construct
  source 1..19: mol_type = other DNA
tttgtttcggtttttggcg

SEQ ID NO: 20 - DNA, 17 bp, synthetic construct
  source 1..17: mol_type = other DNA
cgccaccataaacgacc

SEQ ID NO: 21 - DNA, 32 bp, synthetic construct
  source 1..32: mol_type = other DNA
aggccacggacggcgggaggattttcgatttc

SEQ ID NO: 22 - DNA, 18 bp, synthetic construct
  source 1..18: mol_type = other DNA
aaagggagttttcgggcg

SEQ ID NO: 23 - DNA, 19 bp, synthetic construct
  source 1..19: mol_type = other DNA
acccgatacccccattacc

SEQ ID NO: 24 - DNA, 32 bp, synthetic construct
  source 1..32: mol_type = other DNA
cgcgccgaggcgtacgcaaacctaaaaaattc

SEQ ID NO: 25 - DNA, 21 bp, synthetic construct
  source 1..21: mol_type = other DNA
cggttttcgttcgttttttcg

SEQ ID NO: 26 - DNA, 21 bp, synthetic construct
  source 1..21: mol_type = other DNA
cgtaacttccgccttctacgc

SEQ ID NO: 27 - DNA, 35 bp, synthetic construct
  source 1..35: mol_type = other DNA
aggccacggacggttcgtttatcgggtattttagt

SEQ ID NO: 28 - DNA, 23 bp, synthetic construct
  source 1..23: mol_type = other DNA
cgttcgcgttatttatttcggcg

SEQ ID NO: 29 - DNA, 24 bp, synthetic construct
  source 1..24: mol_type = other DNA
gctcctaattctcatcaacgtcgt

SEQ ID NO: 30 - DNA, 24 bp, synthetic construct
  source 1..24: mol_type = other DNA
cgcgccgagggcggcgttttgcgc

SEQ ID NO: 31 - DNA, 21 bp, synthetic construct
  source 1..21: mol_type = other DNA
ggcgtcgcgttttttagagaa

SEQ ID NO: 32 - DNA, 27 bp, synthetic construct
  source 1..27: mol_type = other DNA
ttccttttcgttcgtataaaatttcgt

SEQ ID NO: 33 - DNA, 31 bp, synthetic construct
  source 1..31: mol_type = other DNA
acggacgcggagatcgggttttagcgatgtt

SEQ ID NO: 34 - DNA, 16 bp, synthetic construct
  source 1..16: mol_type = other DNA
tcgcgtcgtcgttcgt

SEQ ID NO: 35 - DNA, 20 bp, synthetic construct
  source 1..20: mol_type = other DNA
gacgccatctaaaaacgcga

SEQ ID NO: 36 - DNA, 29 bp, synthetic construct
  source 1..29: mol_type = other DNA
aggccacggacgtcgttcgtgtcggtttc

SEQ ID NO: 37 - DNA, 22 bp, synthetic construct
  source 1..22: mol_type = other DNA
gttcggcgtagagtttcgtaga

SEQ ID NO: 38 - DNA, 32 bp, synthetic construct
  source 1..32: mol_type = other DNA
gaaaataaaaatttaaaacttttcgaaacgcg

SEQ ID NO: 39 - DNA, 24 bp, synthetic construct
  source 1..24: mol_type = other DNA
cgcgccgagggtaccgcgacgtcc

SEQ ID NO: 40 - DNA, 24 bp, synthetic construct
  source 1..24: mol_type = other DNA
gttgttttatatatcggcgttcgg

SEQ ID NO: 41 - DNA, 24 bp, synthetic construct
  source 1..24: mol_type = other DNA
actacgactatacacgcttaaccg

SEQ ID NO: 42 - DNA, 29 bp, synthetic construct
  source 1..29: mol_type = other DNA
acggacgcggagggttatcgcgggtttcg

SEQ ID NO: 43 - DNA, 19 bp, synthetic construct
  source 1..19: mol_type = other DNA
gggttaggcgttgggtacg

SEQ ID NO: 44 - DNA, 18 bp, synthetic construct
  source 1..18: mol_type = other DNA
aactcatccgcgacgtcg

SEQ ID NO: 45 - DNA, 29 bp, synthetic construct
  source 1..29: mol_type = other DNA
aggccacggacggacgcccaacaaaaacg

SEQ ID NO: 46 - DNA, 19 bp, synthetic construct
  source 1..19: mol_type = other DNA
gtcgtcgttcgagagggta

SEQ ID NO: 47 - DNA, 23 bp, synthetic construct
  source 1..23: mol_type = other DNA
gaacaaaaacgaacgaacgaaca

SEQ ID NO: 48 - DNA, 28 bp, synthetic construct
  source 1..28: mol_type = other DNA
cgcgccgaggatcggtttcgttcgtttg

SEQ ID NO: 49 - DNA, 21 bp, synthetic construct
  source 1..21: mol_type = other DNA
cgaaacgtaaggatgtcgtcg

SEQ ID NO: 50 - DNA, 27 bp, synthetic construct
  source 1..27: mol_type = other DNA
aatttatatatacaccgcgtctccaac

SEQ ID NO: 51 - DNA, 30 bp, synthetic construct
  source 1..30: mol_type = other DNA
acggacgcggagcgatcccgaatccactac

SEQ ID NO: 52 - DNA, 20 bp, synthetic construct
  source 1..20: mol_type = other DNA
gcgcggaattttaggagtgc

SEQ ID NO: 53 - DNA, 18 bp, synthetic construct
  source 1..18: mol_type = other DNA
cgcgaccccacgataatc

SEQ ID NO: 54 - DNA, 30 bp, synthetic construct
  source 1..30: mol_type = other DNA
aggccacggacgcggggttcgagtgtaaat

SEQ ID NO: 55 - DNA, 16 bp, synthetic construct
  source 1..16: mol_type = other DNA
ttgcggaggacgttgc

SEQ ID NO: 56 - DNA, 21 bp, synthetic construct
  source 1..21: mol_type = other DNA
gaaaaaaaacgatacgccgcc

SEQ ID NO: 57 - DNA, 26 bp, synthetic construct
  source 1..26: mol_type = other DNA
cgcgccgaggcggattcgcgagttcg

SEQ ID NO: 58 - DNA, 25 bp, synthetic construct
  source 1..25: mol_type = other DNA
cgcgatagcgttttttattgtcgcg

SEQ ID NO: 59 - DNA, 21 bp, synthetic construct
  source 1..21: mol_type = other DNA
cgcacgaccgtaaaatactcg

SEQ ID NO: 60 - DNA, 30 bp, synthetic construct
  source 1..30: mol_type = other DNA
acggacgcggaggtcgaaatcgaaacgctc

SEQ ID NO: 61 - DNA, 17 bp, synthetic construct
  source 1..17: mol_type = other DNA
tgcgtatggtgggcgag

SEQ ID NO: 62 - DNA, 24 bp, synthetic construct
  source 1..24: mol_type = other DNA
cctaatttacacgtcaaccaatcg

SEQ ID NO: 63 - DNA, 24 bp, synthetic construct
  source 1..24: mol_type = other DNA
cgcgccgagggcgcgtgcgtttgg

SEQ ID NO: 64 - DNA, 26 bp, synthetic construct
  source 1..26: mol_type = other DNA
ttttcgttgattttattcgagtcgtc

SEQ ID NO: 65 - DNA, 22 bp, synthetic construct
  source 1..22: mol_type = other DNA
gaaccctcttcaaataaaccgc

SEQ ID NO: 66 - DNA, 30 bp, synthetic construct
  source 1..30: mol_type = other DNA
cgcgccgaggcgtttggcgtagatataagc

SEQ ID NO: 67 - DNA, 27 bp, synthetic construct
  source 1..27: mol_type = other DNA
tcgattatgtcgttttagacgttatcg

SEQ ID NO: 68 - DNA, 28 bp, synthetic construct
  source 1..28: mol_type = other DNA
tctacatcgacattctaaaacgactaac

SEQ ID NO: 69 - DNA, 30 bp, synthetic construct
  source 1..30: mol_type = other DNA
aggccacggacgcgcataccatcgacttca

SEQ ID NO: 70 - DNA, 19 bp, synthetic construct
  source 1..19: mol_type = other DNA
cgttgacgcgtagttttcg

SEQ ID NO: 71 - DNA, 18 bp, synthetic construct
  source 1..18: mol_type = other DNA
gtcgaccaaaaacgcgtc

SEQ ID NO: 72 - DNA, 26 bp, synthetic construct
  source 1..26: mol_type = other DNA
cgcgccgaggcgtcccgcaactacaa

SEQ ID NO: 73 - DNA, 20 bp, synthetic construct
  source 1..20: mol_type = other DNA
gttcgagtttaggggtagcg

SEQ ID NO: 74 - DNA, 17 bp, synthetic construct
  source 1..17: mol_type = other DNA
ccgcacaaaaaaccgca

SEQ ID NO: 75 - DNA, 26 bp, synthetic construct
  source 1..26: mol_type = other DNA
aggccacggacgatccgcaaacgccc

SEQ ID NO: 76 - DNA, 24 bp, synthetic construct
  source 1..24: mol_type = other DNA
cgggatagagtcgattaattaggc

SEQ ID NO: 77 - DNA, 18 bp, synthetic construct
  source 1..18: mol_type = other DNA
taacttcccctcgacccg

SEQ ID NO: 78 - DNA, 24 bp, synthetic construct
  source 1..24: mol_type = other DNA
cgcgccgaggcggttcggtagcgg

SEQ ID NO: 79 - DNA, 24 bp, synthetic construct
  source 1..24: mol_type = other DNA
cgttaatttgttagatagagggcg

SEQ ID NO: 80 - DNA, 17 bp, synthetic construct
  source 1..17: mol_type = other DNA
tccgaacaaccgcctac

SEQ ID NO: 81 - DNA, 27 bp, synthetic construct
  source 1..27: mol_type = other DNA
aggccacggacgcgaaaaatcccacgc

SEQ ID NO: 82 - DNA, 22 bp, synthetic construct
  source 1..22: mol_type = other DNA
gttagaagacgttttttcgggg

SEQ ID NO: 83 - DNA, 20 bp, synthetic construct
  source 1..20: mol_type = other DNA
aaaacaaaaatcgaccgcga

SEQ ID NO: 84 - DNA, 37 bp, synthetic construct
  source 1..37: mol_type = other DNA
cgcgccgagggcgttaggatttatttttttttttcga

SEQ ID NO: 85 - DNA, 19 bp, synthetic construct
  source 1..19: mol_type = other DNA
tggagttatcggaaggcga

SEQ ID NO: 86 - DNA, 18 bp, synthetic construct
  source 1..18: mol_type = other DNA
cgaactcccgaaacgacg

SEQ ID NO: 87 - DNA, 28 bp, synthetic construct
  source 1..28: mol_type = other DNA
acggacgcggaggcgcgaacacaaaacg

SEQ ID NO: 88 - DNA, 21 bp, synthetic construct
  source 1..21: mol_type = other DNA
ggaggtttcgcgtttcgatta

SEQ ID NO: 89 - DNA, 18 bp, synthetic construct
  source 1..18: mol_type = other DNA
cgaacgatccccgcctac

SEQ ID NO: 90 - DNA, 27 bp, synthetic construct
  source 1..27: mol_type = other DNA
aggccacggacgattcgcgttcgagcg

SEQ ID NO: 91 - DNA, 19 bp, synthetic construct
  source 1..19: mol_type = other DNA
tcggagtcgagtttagcgc

SEQ ID NO: 92 - DNA, 24 bp, synthetic construct
  source 1..24: mol_type = other DNA
cgaaatcgaaaaaacaaaaaccgc

SEQ ID NO: 93 - DNA, 24 bp, synthetic construct
  source 1..24: mol_type = other DNA
cgcgccgaggcggcgttcgcgatt

SEQ ID NO: 94 - DNA, 19 bp, synthetic construct
  source 1..19: mol_type = other DNA
ggttcgggagaaggttcgg

SEQ ID NO: 95 - DNA, 26 bp, synthetic construct
  source 1..26: mol_type = other DNA
cgaaaaacgacgaaaaacgcaaaaac

SEQ ID NO: 96 - DNA, 37 bp, synthetic construct
  source 1..37: mol_type = other DNA
acggacgcggagcatcgctcgaaaaaaacaaaaaaac

SEQ ID NO: 97 - DNA, 23 bp, synthetic construct
  source 1..23: mol_type = other DNA
agagttcggcgtttagagttagc

SEQ ID NO: 98 - DNA, 30 bp, synthetic construct
  source 1..30: mol_type = other DNA
ttaaatataaaatcgaaacctatatccgcg

SEQ ID NO: 99 - DNA, 38 bp, synthetic construct
  source 1..38: mol_type = other DNA
aggccacggacgcggtttttcgaggatttattattaag

SEQ ID NO: 100 - DNA, 24 bp, synthetic construct
  source 1..24: mol_type = other DNA
tgttatgggttagtgggattcgtc

SEQ ID NO: 101 - DNA, 20 bp, synthetic construct
  source 1..20: mol_type = other DNA
ccgaaaaccacaaatcccgc

SEQ ID NO: 102 - DNA, 26 bp, synthetic construct
  source 1..26: mol_type = other DNA
cgcgccgaggcggattcgcgagttcg

SEQ ID NO: 103 - DNA, 22 bp, synthetic construct
  source 1..22: mol_type = other DNA
agagttggcgagttggttgtac

SEQ ID NO: 104 - DNA, 28 bp, synthetic construct
  source 1..28: mol_type = other DNA
cgaattacaacaaaaccgaataacgcga

SEQ ID NO: 105 - DNA, 28 bp, synthetic construct
  source 1..28: mol_type = other DNA
acggacgcggagcgatacggaaaggcgt

SEQ ID NO: 106 - DNA, 17 bp, synthetic construct
  source 1..17: mol_type = other DNA
gcgggagtttggcgtag

SEQ ID NO: 107 - DNA, 21 bp, synthetic construct
  source 1..21: mol_type = other DNA
cgcgcaaataccgaataaacg

SEQ ID NO: 108 - DNA, 34 bp, synthetic construct
  source 1..34: mol_type = other DNA
aggccacggacggtcggtagatcgttagtagatg

SEQ ID NO: 109 - DNA, 26 bp, synthetic construct
  source 1..26: mol_type = other DNA
agtcgtttttttaggtagtttaggcg

SEQ ID NO: 110 - DNA, 19 bp, synthetic construct
  source 1..19: mol_type = other DNA
cgacctttacaatcgccgc

SEQ ID NO: 111 - DNA, 25 bp, synthetic construct
  source 1..25: mol_type = other DNA
cgcgccgaggggcggtagttgttgc

SEQ ID NO: 112 - DNA, 23 bp, synthetic construct
  source 1..23: mol_type = other DNA
ggagaagagtgcgtaggttagag

SEQ ID NO: 113 - DNA, 22 bp, synthetic construct
  source 1..22: mol_type = other DNA
catatcgcccgacgtaaaaacc

SEQ ID NO: 114 - DNA, 24 bp, synthetic construct
  source 1..24: mol_type = other DNA
cgcgccgaggctcgcgaaacgccg

SEQ ID NO: 115 - DNA, 29 bp, synthetic construct
  source 1..29: mol_type = other DNA
aggccacggacgggttatcgcgggtttcg

SEQ ID NO: 116 - DNA, 29 bp, synthetic construct
  source 1..29: mol_type = other DNA
cgcgccgaggatcgggttttagcgatgtt

SEQ ID NO: 117 - DNA, 35 bp, synthetic construct
  source 1..35: mol_type = other DNA
cgcgccgaggcatcgctcgaaaaaaacaaaaaaac

SEQ ID NO: 118 - DNA, 26 bp, synthetic construct
  source 1..26: mol_type = other DNA
cgcgccgagggcgcgaacacaaaacg

SEQ ID NO: 119 - DNA, 30 bp, synthetic construct
  source 1..30: mol_type = other DNA
aggccacggacggtcgaaatcgaaacgctc

SEQ ID NO: 120 - DNA, 26 bp, synthetic construct
  source 1..26: mol_type = other DNA
cgcgccgaggcgatacggaaaggcgt

SEQ ID NO: 121 - DNA, 28 bp, synthetic construct
  source 1..28: mol_type = other DNA
cgcgccgaggcgatcccgaatccactac

(19) United States
(12) Patent Application Publication
(10) Pub. No.: US 2025/0333781 A1
(43) Pub. Date: Oct. 30, 2025

(54) LOW-BIAS SEQUENTIAL MULTIPLEX AMPLIFICATION ASSAYS

(71) Applicant: Exact Sciences Corporation, Madison, WI (US)

(72) Inventors: Gracie Shea, Madison, WI (US); Abram M. Vaccaro, Rio, WI (US); Cynthia Moehlenkamp, Verona, WI (US); Hatim T. Allawi, Middleton, WI (US); Viatcheslav Katerov, Madison, WI (US); Michael W. Kaiser, Stoughton, WI (US)

(21) Appl. No.: 18/861,043

(22) PCT Filed: Apr. 27, 2023
(86) PCT No.: PCT/US2023/066278

§ 371 Date: Oct. 28, 2024

Related U.S. Application Data
(60) Provisional application No. 63/335,360, filed on Apr. 27, 2022.

(51) Int. Cl. C12Q 1/686

(52) U.S. Cl. CPC C12Q 1/686

(57) ABSTRACT

Provided herein is technology relating to multiplex preamplification of multiple different target nucleic acids from a sample, e.g., an environmental or biological sample, and particularly, but not exclusively, to methods, compositions, kits, and related uses for detecting and assessing mixtures of target nucleic acids, e.g.. The technology provides methods, compositions, and kits for using low-bias amplification conditions to perform highly-multiplexed preamplification followed by highly-multiplexed detection assays such as PCR assays, next generation sequencing assays, and PCR-flap assay for assaying samples that comprise multiple different target nucleic acids.

### SEQUENCE LISTING

The text of the computer readable sequence listing filed herewith, titled “39883-601_SEQUENCE_LISTING”, created Apr. 27, 2023, having a file size of 108,041 bytes, is hereby incorporated by reference in its entirety.

### FIELD OF THE INVENTION

Provided herein is technology relating to the amplification-based detection of nucleic acids and particularly, but not exclusively, to methods and compositions for serial steps of multiplex amplification to balance amplification from different target nucleic acids present in the reaction mixture. The technology further provides methods for using combined signal from serial multiplex amplification of multiple marker genes in a single fluorescence channel without differentiating signal from any single marker gene in the combination.

### BACKGROUND OF THE INVENTION

Methods for detection and quantification of nucleic acids are important in many areas of molecular biology and in particular for molecular diagnostics. At the DNA level, such methods are used, for example, to determine the presence or absence of variant alleles, the copy numbers of gene sequences amplified in a genome, and the amount, presence, or absence of methylation across genes or at specific loci within genes. Further, methods for the quantification of nucleic acids are used to determine mRNA quantities as a measure of gene expression.

Among the number of different analytical methods that detect and quantify nucleic acids or nucleic acid sequences, variants of the polymerase chain reaction (PCR) have become the most powerful and widespread technology, the principles of which are disclosed in U.S. Pat. Nos. 4,683,195; 4,683,202; and 4,965,188. Preamplification of target nucleic acids (e.g., genomic DNA, cDNA, etc.) in a low-target sample may be used to enrich the DNA in the sample prior to dividing the sample for further specific target analysis. For example, whole genome amplification using simple primers (e.g., random hexamers) has been used to increase the amounts of essentially all DNA in a sample, in a manner that is not specific to any particular target of interest. (Sigma-Aldrich's GenomePlex systems, Arneson, et al., Cold Spring Harb. Protoc.; 2008; doi:10.1101/pdb.prot4920).

Another approach is to amplify one or more regions of particular interest in a semi-targeted manner, to produce a mixture of amplified fragments (amplicons) that contains the different mutations or loci that will be further analyzed. Successive rounds of amplification using the same primers are prone to high background of non-specific amplification, and the production of artifacts, e.g., artificially recombined molecules, high non-specific background, and biased amplification of different intended targets. Thus, such preamplification PCR is typically carried out under special conditions e.g., a limited number of cycles, and/or using a low concentration of primers (e.g., 10 to 20-fold lower than in standard PCR) to avoid increases in non-specific background amplification, as use of concentrations over about 160 nM of each primer in multiplex preamplification has been shown to increase amplification background in negative control reactions (see, e.g., Andersson, et al., Expert Rev. Mol. Diagn. Early online, 1-16 (2015)).

After a first round of amplification in a multiplex PCR, preamplified DNA is typically diluted and aliquoted into new amplification reactions for quantitative or qualitative PCR analysis using conditions typical of standard PCR, e.g., higher concentrations of reagents and larger numbers of cycles, and the second amplification is generally carried out using different primer pairs, e.g., “nested” primers that anneal to sites within the preamplified fragments, rather than annealing to the original primer sites at the ends of the amplicons.

Some uses of amplification involve measurement or analysis of multiple mutations or marker nucleic acids in a sample. Multiplex amplification of a plurality of different specific target sequences is typically conducted using relatively standard PCR reagent mixtures, e.g., for Amplitaq DNA polymerase, mixtures comprising 50 mM KCl, 1.5 to 2.5 mM MgCl₂, and Tris-HCl buffer at about pH 8.5 are used. If a second amplification is to be performed, the primers are typically present in limited amounts (see Andersson, supra). For a subsequent assay, the amplified DNA is diluted or purified, and a small aliquot is then added to an additional amplification reaction.

Re-amplifying DNA segments previously amplified in a targeted manner, e.g., amplification of an aliquot or dilution of the amplicon product of a target-specific PCR, is known to be prone to undesirable artifacts, e.g., high background of undesired DNA products. Thus, analysis of target nucleic acids using sequential rounds of specific PCR is typically conducted under special conditions, e.g., using different primers pairs in the sequential reactions. For example, in “nested PCR” the first round of amplification is conducted to produce a first amplicon, and the second round of amplification is conducted using a primer pair in which one or both of the primers anneal to sites inside the regions defined by the initial primer pair, i.e., the second primer pair is considered to be “nested” within the first primer pair. In this way, background amplification products from the first PCR that do not contain the correct inner sequence are not further amplified in the second reaction. Other strategies to reduce undesirable effects include using very low concentrations of primers in the first amplification. A change in reaction conditions between a first amplification and a second amplification (or other detection assay) is often effected by either purifying the DNA from the first amplification reaction or by using sufficient dilution such that the amounts of reaction components carried into the follow-on reaction is negligible.

### SUMMARY OF THE INVENTION

In the course of development of methods described herein, it has been determined that complex combinations of marker nucleic acids may be both preamplified and then amplified for real-time detection without the need for individually optimizing concentrations of different individual primer pairs to bring amplification efficiencies into alignment. Conditions are provided that reduce amplification bias between different co-amplified targets in complexly multiplexed preamplification mixtures.

Surprisingly, use of preamplification reaction conditions that reduce amplification bias among the multiplexed targets allows further complex multiplexing in follow-on PCR assays, such as PCR-flap assay reactions, removing the need to use differently labeled probes or FRET cassettes to separately detect and measure each different target that is amplified in the follow-on detection reaction, and allowing, for example, analyses based on the composite signal without the need to measure each signal separately.

The technology does not require either whole-genome preamplification and or the use of nested primers or nested primer pairs in the PCR-flap assay reaction. Surprisingly, the targeted preamplification can be multiplexed using a combination of the same primer pairs that will be used in a second round of highly multiplexed amplification of the same set of targets (or a subset of the target loci). In preferred embodiments, follow-on multiplexed detection assays comprise PCR-flap assay reactions, e.g., QuARTS and LQAS/TELQAS flap assay reactions, which combine PCR target amplification and FEN-1-mediated flap cleavage for signal amplification.

The methods, compositions, systems, devices, and kits disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

The technology provides but is not limited to the enumerated embodiments below:

1. A method of analyzing a mixture comprising multiple target nucleic acids, comprising:
a) treating a sample suspected of comprising multiple different target nucleic acids in a preamplification reaction mixture to produce a multiplex preamplified mixture,
   wherein the preamplification reaction mixture comprises at least 4 different target-specific primer pairs for producing amplified regions from at least 4 different target nucleic acids, if present in the sample, and at least one reference primer pair for producing an amplified region from a reference nucleic acid;
   wherein each of the primers in the at least 4 different target-specific primer pairs and the at least one reference primer pair are in essentially the same concentrations;
b) adding a portion of the multiplex preamplified mixture to a multiplex PCR assay reaction mixture comprising:
   i) additional amounts of each of the at least 4 different target-specific primer pairs and the reference primer pair, wherein the primers in the additional amounts of the at least 4 different target-specific primer pairs and the at least one reference primer pair are added in essentially the same amounts;
   ii) at least 4 different target-specific probe oligonucleotides, wherein each target-specific probe oligonucleotide specifically hybridizes to a different one of the amplified regions from the at least 4 different target nucleic acids, if amplified in step a), wherein the at least 4 different target-specific probe oligonucleotides comprise a first label; and
   iii) a reference probe flap oligonucleotide that specifically hybridizes to the amplified region from the reference nucleic acid, wherein the reference probe flap oligonucleotide comprises a second label;
and
c) conducting a PCR assay with said multiplex PCR assay reaction mixture, wherein the reference nucleic acid region and each of said at least 4 different target regions, if amplified in step a), are amplified in the PCR assay reaction mixture, wherein target-specific probe oligonucleotides and the reference probe oligonucleotide specifically hybridize to target regions and the reference nucleic acid region amplified in the multiplex PCR assay reaction mixture and are cleaved to release first and second labels, wherein said released first and second labels are measured.

2. The method of embodiment 1, wherein the preamplification reaction mixture comprises a low-bias amplification buffer.

3. The method of embodiment 2, wherein the multiplex PCR assay reaction mixture comprises a low-bias amplification buffer, preferably the same low-bias amplification buffer used in the preamplification reaction mixture.

4. The method of any one of embodiments 1-3, wherein in step b) the at least four different target-specific probe oligonucleotides and the reference probe flap oligonucleotide are present in said multiplex PCR assay reaction mixture in essentially the same concentrations.

5. The method of any one of embodiments 1-4, wherein said first label comprises a first 5′ flap sequence, wherein the first 5′ flap sequence is not substantially complementary to any of the amplified regions from the at least 4 different target nucleic acids.

6. The method of embodiment 5, wherein said second label comprises a second 5′ flap sequence, wherein the second 5′ flap sequence is different than the first 5′ flap sequence and is not substantially complementary to the amplified region from the reference nucleic acid.

7. The method of embodiment 5 or embodiment 6, wherein the PCR assay reaction mixture further comprises a first FRET cassette labeled with a first fluorophore, the first FRET cassette comprising a sequence complementary to the first 5′ flap sequence, and/or a second FRET cassette labeled with a second fluorophore, the second FRET cassette comprising a sequence complementary to the second 5′ flap sequence.

8. The method of any one of embodiments 1-7, wherein the PCR assay reaction mixture further comprises a flap endonuclease, preferably a FEN-1 endonuclease, preferably an archaeal FEN-1 endonuclease.

9. The method of any one of embodiments 1-8, wherein said first label comprises a first FRET system comprising a first fluorophore, and wherein said second label comprises a second FRET system comprising a second fluorophore, and wherein fluorescence from the first fluorophore and the second fluorphore is measured during said PCR assay.

10. The method of any one of embodiments 1-9, wherein the at least 4 different target-specific primer pairs comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different target-specific primer pairs.

11. The method of any one of embodiments 1-10, wherein the at least 4 different target-specific probe oligonucleotides comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different target-specific probe oligonucleotides.

12. The method of any one of embodiments 1-11, wherein the concentrations of the at least four different target-specific primer pairs and the reference primer pair in the PCR assay reaction mixture are essentially the same as the concentrations of the at least four different target-specific primer pairs and the reference primer pair in the preamplification reaction mixture.

13. The method of any one of embodiments 1-12, wherein the low-bias amplification buffer comprises 3-(n-morpholino) propanesulfonic acid (MOPS) buffer and at least about 6 mM, preferably 6.1, 6.2, 6.5, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, or 11.0 mM Mg⁺⁺.

14. The method of embodiment 13, wherein the low-bias amplification buffer comprises about 7.5 mM Mg⁺⁺.

15. The method of any one of embodiments 1-14, wherein the preamplification reaction mixture comprises at least one additional target-specific primer pair for producing an amplified region from an additional target nucleic acid, if present in the sample, that is different from the at least four different target nucleic acids and from the reference nucleic acid, and wherein the multiplex PCR assay reaction mixture further comprises:
i) an additional amount of at the at least 1 additional target-specific primer pair in essentially the same amount as the additional amounts of the at least four different target-specific primer pairs; and
ii) at least 1 additional target-specific probe oligonucleotide that specifically hybridizes to an amplified region from the at least 1 additional target nucleic acid, if amplified in step a), the at least 1 additional target-specific probe oligonucleotide having a third label, wherein the third label is different than the first and the second label.

16. The method of embodiment 15, wherein said third label comprises a third 5′ flap sequence, wherein the third 5′ flap sequence is different than the first 5′ flap sequence and the second 5′ flap sequence and is not substantially complementary to the amplified region from the additional target nucleic acid.

17. The method of embodiment 16, wherein the PCR assay reaction mixture further comprises a third FRET cassette labeled with a third fluorophore, the third FRET cassette comprising a sequence complementary to the third 5′ flap sequence.

18. The method of any one of embodiments 14-17, wherein the at least 1 additional target-specific primer pair comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different additional target-specific primer pairs for producing amplified regions from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different additional target nucleic acids, if present in the sample; and
wherein the multiplex PCR assay reaction mixture further comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 additional target-specific probe oligonucleotides comprising the third label, wherein the additional target-specific probe oligonucleotides specifically hybridize to amplified regions from the 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different additional target nucleic acids, if amplified in step a).

19. A method of analyzing a sample for multiple target nucleic acids in a PCR-flap assay, comprising:
a) treating nucleic acid sample in a preamplification reaction mixture, to produce a multiplex preamplified mixture,
   wherein the preamplification reaction mixture comprises at least 4 different target-specific primer pairs for producing amplified regions from at least 4 different target nucleic acids, if present in the sample, and at least one reference primer pair for producing an amplified region from a reference nucleic acid;
   wherein each of the primers in the at least 4 different target-specific primer pairs and the at least one reference primer pair are in essentially the same concentrations;
b) adding a portion of the multiplex preamplified mixture to a multiplex PCR-flap assay reaction mixture comprising:
   i) additional amounts of each of the at least 4 different target-specific primer pairs and the reference primer pair, wherein the primers in the additional amounts of the at least 4 different target-specific primer pairs and the at least one reference primer pair are added in essentially the same amounts;
   ii) at least 4 different target-specific flap probe oligonucleotides, wherein each target-specific flap probe oligonucleotide specifically hybridizes to a different one of the amplified regions from the at least 4 different target nucleic acids, if amplified in step a), wherein each one of the at least 4 different target-specific flap probe oligonucleotides comprises a first 5′ flap sequence, wherein the first 5′ flap sequence is not substantially complementary to any of the amplified regions from the at least 4 different target nucleic acids;
   iii) a reference flap probe oligonucleotide that specifically hybridizes to the amplified region from the reference nucleic acid, the reference flap probe oligonucleotide having a second 5′ flap sequence, wherein the second 5′ flap sequence is different than the first 5′ flap sequence and is not substantially complementary to the amplified region from the reference nucleic acid;
   iv) a first FRET cassette labeled with a first fluorophore, the first FRET cassette comprising a sequence complementary to the first 5′ flap sequence;
   v) a second FRET cassette labeled with a second fluorophore, the second FRET cassette comprising a sequence complementary to the second 5′ flap sequence; and
   vi) a PCR-flap assay buffer;
and
c) conducting a PCR-flap assay with said multiplex PCR-flap assay reaction mixture, wherein the reference nucleic acid and each of said at least 4 different target regions, if amplified in step a), are amplified in the PCR-flap assay reaction mixture, and fluorescence from the first fluorophore and the second fluorphore are measured.

20. The method of embodiment 19, wherein the preamplification reaction mixture comprises a low-bias amplification buffer.

21. The method of embodiment 19 or embodiment 20, wherein in step b) the at least 4 different target-specific flap probe oligonucleotides and the reference flap probe oligonucleotide present in said multiplex PCR-flap assay reaction mixture are in essentially the same concentrations.

22. The method of any one of embodiments embodiment 1-21, wherein treating the nucleic acid in the preamplification reaction mixture comprises thermal cycling the preamplification reaction mixture for fewer than 20 thermal cycles, preferably fewer than 19, 18, 17, 16, 15, 14, 13, 12, 11, 10, 9, 8, 7, 6, or 5 thermal cycles.

23. The method of any one of embodiments 19-21, wherein the concentrations of the at least four different target-specific primer pairs and the reference primer pair in the PCR-flap assay reaction mixture are essentially the same as the concentrations of the at least four different target-specific primer pairs and the reference primer pair in the preamplification reaction mixture.

24. The method of any one of embodiments 1-23, further comprising diluting at least a portion of the multiplex preamplified mixture prior to step b).

25. The method of any one of embodiments 19-24, wherein the preamplification reaction mixture comprises at least one additional target-specific primer pair for producing an amplified region from an additional target nucleic acid, if present in the sample, that is different from the at least four different target nucleic acids and from the reference nucleic acid, and wherein the multiplex PCR-flap assay reaction mixture further comprises:
i) an additional amount of the at least 1 additional target-specific primer pair in essentially the same amount as the additional amounts of the at least four different target-specific primer pairs;
ii) at least 1 additional target-specific flap probe oligonucleotide that specifically hybridizes to an amplified region from the at least 1 additional target nucleic acid, if amplified in step a), the at least 1 additional target-specific flap probe oligonucleotide having a third 5′ flap sequence, wherein the third 5′ flap sequence is different than the first and the second 5′ flap sequences and is not substantially complementary to the amplified region from the at least 1 additional target nucleic acid; and
iii) a third FRET cassette labeled with a third fluorophore, the third FRET cassette comprising a sequence complementary to the third 5′ flap sequence.

26. The method of any one of embodiments 19-25, wherein the at least 1 additional target-specific primer pair comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different additional target-specific primer pairs for producing amplified regions from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different additional target nucleic acids, if present in the sample; and wherein the multiplex PCR-flap assay reaction mixture further comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 additional target-specific flap probe oligonucleotides comprising the third label, wherein the additional target-specific flap probe oligonucleotides specifically hybridize to amplified regions from the 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different additional target nucleic acids, if amplified in step a).

27. The method of any one of embodiments 1-26, wherein the sample suspected of comprising multiple different target nucleic acids is prepared from a sample comprising one or more of soil, water, plant material, an animal or human sample comprising one or more of stool, tissue, sputum, mucus, blood or a blood product selected from plasma, serum, whole blood, an organ excretion, and urine.

28. The method of embodiment 27, wherein the sample suspected of comprising multiple different target nucleic acids comprises cell-free DNA isolated from plasma.

29. The method of embodiment 28, wherein the sample suspected of comprising multiple different target nucleic acids comprises cDNA prepared from RNA isolated from plasma.

30. The method of embodiment 28 or embodiment 29, wherein the preamplification reaction mixture of step a) has a total volume, wherein the sample suspected of comprising multiple different target nucleic acids is prepared from at least one mL of plasma, and is at least 20 to 50% of the total volume of the preamplification reaction mixture of step a).

31. A method of analyzing a sample for at least 10 different target nucleic acids in a single PCR-flap assay reaction, the method comprising:
a) treating nucleic acid comprising multiple different target nucleic acids in a preamplification reaction mixture comprising a PCR-flap assay buffer, to produce a multiplex preamplified mixture,
   wherein the preamplification reaction mixture comprises at least 10 different target-specific primer pairs for producing amplified regions from at least 10 different target nucleic acids in the sample and at least one reference primer pair for producing an amplified region from a reference nucleic acid, wherein each of the primers in the at least 10 different target-specific primer pairs and the at least one reference primer pair are in essentially the same concentration in the preamplification reaction mixture;
b) adding a portion of the multiplex preamplified mixture to a multiplex PCR-flap assay reaction mixture comprising:
   i) additional amounts of each of the at least 10 different target-specific primer pairs and the reference primer pair, wherein the primers in the additional amounts of the at least 10 different target-specific primer pairs and the at least one reference primer pair are in essentially the same concentration in the multiplex PCR-flap assay reaction mixture;
   ii) at least 10 different target-specific flap oligonucleotides, wherein each target-specific flap oligonucleotide specifically hybridizes to a different one of the amplified regions from the at least 10 different target nucleic acids,
      wherein the at least 10 different target-specific flap oligonucleotides are divided into a first group and a second group, wherein each one of the flap oligonucleotides in the first group comprises a first 5′ flap sequence, and wherein each one of the flap oligonucleotides in the second group comprises a second 5′ flap sequence;
   iii) a reference flap oligonucleotide that specifically hybridizes to the amplified region from the reference nucleic acid, the reference flap oligonucleotide having a third 5′ flap sequence, wherein the third 5′ flap sequence is different than the first 5′ flap sequence and the second 5′ flap sequence;
   iv) a first FRET cassette labeled with a first fluorophore, the first FRET cassette comprising a sequence complementary to the first 5′ flap sequence;
   v) a second FRET cassette labeled with a second fluorophore, the second FRET cassette comprising a sequence complementary to the second 5′ flap sequence;
   vi) a third FRET cassette labeled with a third fluorophore, the third FRET cassette comprising a sequence complementary to the third 5′ flap sequence; and
   vi) PCR-flap assay buffer;
and
c) conducting a PCR-flap assay with said multiplex PCR-flap assay reaction mixture, wherein the reference nucleic acid and each of said at least 10 different target regions are amplified in the PCR-flap assay reaction mixture, and fluorescence from the first fluorophore, the second fluorphore, and the third fluorphore are measured.

32. The method of embodiment 31, wherein conducting the PCR-flap assay with said multiplex PCR-flap assay reaction mixture comprises thermal cycling the multiplex PCR-flap assay reaction mixture for at least 25 thermal cycles, preferably more than 30, 31, 32, 33, 34, 35, 36, 37, 38, 39, 40, 41, 42, 43, 44, or 45 thermal cycles.

33. The method of embodiment 31 or embodiment 32, wherein fluorescence from the first fluorophore, the second fluorphore, and the third fluorphore is measured during thermal cycling.

34. A composition comprising in a mixture:
a) a group of oligonucleotides comprising:
   i) a first set of at least four different target-specific primer pairs for producing amplified regions from a first group of at least four different target nucleic acids;
   ii) at least one reference primer pair for producing an amplified region from a reference nucleic acid;
   wherein each of the primers in the at least four different target-specific primer pairs and the at least one reference primer pair are in essentially the same concentration;
   iii) a first set of at least four different target-specific flap oligonucleotides, wherein each target-specific flap oligonucleotide specifically hybridizes to a different one of the amplified regions from the group of at least four different target nucleic acids,
   wherein each one of the at least four different flap oligonucleotides comprises a first 5′ flap sequence;
   iv) a reference flap oligonucleotide that specifically hybridizes to the amplified region from the reference nucleic acid, the reference flap oligonucleotide having a second 5′ flap sequence, wherein the second 5′ flap sequence is different than the first 5′ flap sequence;
   v) a first FRET cassette labeled with a first fluorophore, the first FRET cassette comprising a sequence complementary to the first 5′ flap sequence and not substantially complementary to the second 5′ flap sequence; and
   vi) a second FRET cassette labeled with a second fluorophore, the second FRET cassette comprising a sequence complementary to the second 5′ flap sequence and not substantially complementary to the first 5′ flap sequence; and
b) dNTPs.

35. The composition of embodiment 34, wherein the first set of at least four different target-specific flap oligonucleotides comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different flap oligonucleotides.

36. The composition of embodiment 34 or embodiment 35, wherein the first set of at least four different target-specific primer pairs comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different primer pairs.

37. The composition of any one of embodiments 34-36, further comprising:
vii) a second set of different target-specific primer pairs for producing amplified regions from a second group of different target nucleic acids;
wherein each of the primers in the second set of different target-specific primer pairs are in essentially the same amount or concentration as the amount or concentration of the primers in the first set of target-specific primer pairs;
viii) a second set of different target-specific flap oligonucleotides, wherein each target-specific flap oligonucleotide specifically hybridizes to a different one of the amplified regions from the second group of different target nucleic acids,
wherein each one of the flap oligonucleotides in the second set of target-specific flap oligonucleotides comprises a third 5′ flap sequence; and
ix) a third FRET cassette labeled with a third fluorophore, the third FRET cassette comprising a sequence complementary to the third 5′ flap sequence and not substantially complementary to the first 5′ flap sequence or the second 5′ flap sequence.

38. The composition of embodiment 37, wherein the second set of different target-specific flap oligonucleotides comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different flap oligonucleotides.

39. The composition of embodiment 37 or embodiment 38, wherein the second set of different target-specific primer pairs comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different primer pairs.

40. The composition of any one of embodiments 37-39, further comprising one or more of:
x) a DNA polymerase, preferably a thermostable DNA polymerase;
xi) a flap endonuclease, preferably a FEN-1 endonuclease, preferably an archaeal FEN-1 endonuclease; and
xii) a low-bias amplification buffer.

41. The composition of any one of embodiments 37-40, further comprising:
xiii) a portion of a multiplex preamplified mixture amplified in a low-bias amplification buffer using at least four different target-specific primer pairs and at least one reference primer pair in essentially the same concentrations, the multiplex preamplified mixture comprising amplified regions of the first group of at least four different target nucleic acids and the reference nucleic acid.

42. A kit comprising:
a) a mixture comprising a group of oligonucleotides comprising:
   i) a first set of at least four different target-specific primer pairs for producing amplified regions from a first group of at least four different target nucleic acids in the sample;
   ii) at least one reference primer pair for producing an amplified region from a reference nucleic acid in the sample;
   wherein each of the primers in the at least four different target-specific primer pairs and the at least one reference primer pair are in essentially the same amount or concentration;
   iii) a first set of at least four different target-specific flap oligonucleotides, wherein each target-specific flap oligonucleotide specifically hybridizes to a different one of the amplified regions from the group of at least four different target nucleic acids,
   wherein each one of the at least four different flap oligonucleotides comprises a first 5′ flap sequence;
   iv) a reference flap oligonucleotide that specifically hybridizes to the amplified region from the reference nucleic acid, the reference flap oligonucleotide having a second 5′ flap sequence,
   wherein the second 5′ flap sequence is different than the first 5′ flap sequence;
   v) a first FRET cassette labeled with a first fluorophore, the first FRET cassette comprising a sequence complementary to the first 5′ flap sequence and not substantially complementary to the second 5′ flap sequence; and
   vi) a second FRET cassette labeled with a second fluorophore, the second FRET cassette comprising a sequence complementary to the second 5′ flap sequence and not substantially complementary to the first 5′ flap sequence.

43. The kit of embodiment 42, wherein the first set of at least four different target-specific flap oligonucleotides comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different flap oligonucleotides.

44. The kit of embodiment 42 or embodiment 43, wherein the first set of at least four different target-specific primer pairs comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different primer pairs.

45. The kit of any one of embodiments 42-44, wherein the mixture further comprising:
vii) a second set of different target-specific primer pairs for producing amplified regions from a second group of different target nucleic acids in a sample;
wherein each of the primers in the second set of different target-specific primer pairs are in essentially the same amount or concentration as the amount or concentration of the primers in the first set of target-specific primer pairs;
viii) a second set of different target-specific flap oligonucleotides, wherein each target-specific flap oligonucleotide specifically hybridizes to a different one of the amplified regions from the second group of different target nucleic acids,
wherein each one of the flap oligonucleotides in the second set of target-specific flap oligonucleotides comprises a third 5′ flap sequence; and
ix) a third FRET cassette labeled with a third fluorophore, the third FRET cassette comprising a sequence complementary to the third 5′ flap sequence and not substantially complementary to the first 5′ flap sequence or the second 5′ flap sequence.

46. The kit of embodiment 45, wherein the second set of different target-specific flap oligonucleotides comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different flap oligonucleotides.

47. The kit of embodiment 45 or embodiment 46, wherein the second set of different target-specific primer pairs comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different primer pairs.

48. The kit of any one of embodiments 42-47, further comprising one or more of
b) a DNA polymerase, preferably a thermostable DNA polymerase;
c) a flap endonuclease, preferably a FEN-1 endonuclease, preferably an archaeal FEN-1 endonuclease;
d) a low-bias amplification buffer; and
e) dNTPs.

49. The kit of any one of embodiments 42-48, further comprising:
f) in a second mixture, portions of the group of oligonucleotides comprising:
   i) the first set of at least four different target-specific primer pairs for producing amplified regions from a first group of at least four different target nucleic acids in a sample;
   ii) the at least one reference primer pair for producing an amplified region from a reference nucleic acid in the sample;
   wherein each of the primers in the at least four different target-specific primer pairs and the at least one reference primer pair are in essentially the same amount or concentration.

50. The kit of any one of embodiments 42-49, wherein the mixture of a) is in dried form or in the form of a solution.

51. The kit of embodiment 49 or 50, wherein the mixture of f) is in dried form or in the form of a solution.

52. The kit of any one of embodiments 48-51, wherein the low-bias amplification buffer comprises 3-(n-morpholino) propanesulfonic acid (MOPS) buffer and a concentration of Mg⁺⁺ to provide a final concentration of Mg⁺⁺ in a PCR reaction mixture of at least about 6 mM, preferably 6.1, 6.2, 6.5, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, or 11.0 mM Mg⁺⁺.

53. The method of embodiment 52, wherein the low-bias amplification buffer comprises a concentration of Mg⁺⁺ to provide a final concentration of Mg⁺⁺ in a PCR reaction mixture of about 7.5 mM Mg⁺⁺.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The term “gene” refers to a nucleic acid (e.g., DNA or RNA) sequence that comprises coding sequences necessary for the production of an RNA, or of a polypeptide or its precursor. A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a “gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and poly adenylation.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “wild-type” when made in reference to a protein refers to a protein that has the characteristics of a naturally occurring protein. The term “naturally-occurring” as applied to an object refers to the fact that an object can be found in nature. For example, a polypeptide or polynucleotide sequence that is present in an organism (including viruses) that can be isolated from a source in nature, and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population, or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state”, “methylation profile”, and “methylation status” of a nucleic acid molecule refers to the presence or absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated. In some embodiments, a nucleic acid may be characterized as “unmethylated” if it is not methylated at a specific locus (e.g., the locus of a specific single CpG dinucleotide) or specific combination of loci, even if it is methylated at other loci in the same gene or molecule.

The methylation state of a particular nucleic acid sequence (e.g., a gene marker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs.

As used herein, the terms “marker gene,” “biomarker” and “marker” are used interchangeably to refer to DNA, RNA, or protein (or other sample components) that is associated with a condition of a subject or environment from which a sample is obtained. A biomarker may be indicative of, e.g., a strain of virus or bacteria present in an environment or a subject, a cancer or other gene-related disease, regardless of whether the marker region is in a coding region of DNA. Markers may include, e.g., regulatory regions, flanking regions, intergenic regions, etc. Similarly, the term “marker” used in reference to any component of a sample, e.g., protein, RNA, carbohydrate, small molecule, etc., refers to a component that can be assayed in a sample (e.g., measured or otherwise characterized) and that is associated with a condition of a subject, or of the sample from a subject. The term “methylation marker” refers to a gene or DNA in which the methylation state of the gene or DNA is associated with a condition, e.g., cancer.

The methylation state of a nucleotide locus in a nucleic acid molecule refers to the presence or absence of a methylated nucleotide at a particular locus in the nucleic acid molecule. For example, the methylation state of a cytosine at the 7th nucleotide in a nucleic acid molecule is methylated when the nucleotide present at the 7th nucleotide in the nucleic acid molecule is 5-methylcytosine. Similarly, the methylation state of a cytosine at the 7th nucleotide in a nucleic acid molecule is unmethylated when the nucleotide present at the 7th nucleotide in the nucleic acid molecule is cytosine (and not 5-methylcytosine).

The methylation status can optionally be represented or indicated by a “methylation value” (e.g., representing a methylation frequency, fraction, ratio, percent, etc.) A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, “methylation frequency” or “methylation percent (%)” refer to the number of instances in which a molecule or locus is methylated relative to the number of instances the molecule or locus is unmethylated.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “unmethylated”, “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides is the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who do not have recurrence. Differential methylation and specific levels or patterns of DNA methylation are prognostic and predictive biomarkers, e.g., once the correct cut-off or predictive characteristics have been defined.

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

As used herein a “nucleotide locus” refers to the location of a nucleotide in a nucleic acid molecule. A nucleotide locus of a methylated nucleotide refers to the location of a methylated nucleotide in a nucleic acid molecule.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g., Antequera, et al. (1990) Cell 62: 503-514).

As used herein, a “CpG island” refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55% and the ratio of observed CpG frequency over expected frequency is 0.65. The observed CpG frequency over expected frequency can be calculated according to the method provided in Gardiner-Garden et al (1987) J. Mol. Biol. 196: 261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see Ramsahoye (2000) Proc. Nat. Acad. Sci. USA 97: 5237-5242; Salmon and Kaye (1970) Biochim. Biophys. Acta. 204: 340-351; Grafstrom (1985) Nucleic Acids Res. 13: 2827-2842; Nyce (1986) Nucleic Acids Res. 14: 4353-4367; Woodcock (1987) Biochem. Biophys. Res. Commun. 145: 888-894).

As used herein, the terms “methyl cytosine,” “methyl C,” “methylated cytosine,” “methylated C,” and “meC” are used interchangeably and encompass both 5-methylcytosine (5mC) and 5-hydroxymethyl cytosine (5hmC).

As used herein, the term “modified cytosine” or “modified C” refers to a cytosine nucleobase in which the base portion has a side group or other modification as compared to a standard cytosine nucleotide. Modified cytosines include but are not limited to 5-methylcytosine (5mC), 5-hydroxymethylcytosine (5hmC), 5-carboxylcytosine (ScaC), and 5-formylcytosine (5fC).

As used herein, a “methylation-specific reagent” refers to a reagent that modifies a nucleotide of the nucleic acid molecule as a function of the methylation state of the nucleic acid molecule. In particular embodiments the term refers to a compound or composition or other agent or collection or sequence thereof that can change the nucleotide sequence of a nucleic acid molecule in a manner that reflects the methylation state of the nucleic acid molecule. Methods of treating a nucleic acid molecule with such reagents can include contacting the nucleic acid molecule with the reagent, coupled with additional steps, if desired, to accomplish the desired change of nucleotide sequence. Such methods can be applied in a manner in which unmethylated nucleotides (e.g., each unmethylated cytosine) is modified to a different nucleotide. For example, in some embodiments, such a reagent can deaminate unmethylated cytosine nucleotides to produce deoxy uracil residues. An exemplary reagent is a bisulfite reagent.

In addition, treatment with a “methylation-specific reagent” can be applied in a manner in which methylated nucleotides are modified to a different nucleotide. For example, methylated cytosines (including 5mC and 5hmC) in DNA can be converted by combining oxidation by ten-eleven translocation (TET) family dioxygenases with reduction by borane derivatives (e.g., pyridine borane and 2-picoline borane (pic-BH₃)), in a process referred to herein as TAPS (TET Assisted Pyridine borane Sequencing). See, e.g., the TAPS method combining oxidation by TET enzymes with reduction by borane derivatives, described, e.g., in US 2020/0370114 A1, application Ser. No. 16/960,510, filed Jul. 7, 2020, which is incorporated herein by reference for all purposes. In embodiments of the TAPS method, methylated cytosines are converted to dihydro uracil. Other methods of converting methylated Cs include, for example:

| | Cytosine | |
|---|---|---|
| Sequencing Method | Modification | Method of Analysis |
| TET-assisted bisulfite | 5 hmC | Enzymatic treatment with |
| sequencing (TAB-seq) | | T4-BGT then TET followed |
| | | by bisulfite treatment |
| Oxidative bisulfite | 5 mC | Treatment with an |
| sequencing (oxBS) | | oxidation reagent |
| | | followed by bisulfite |
| | | treatment |
| APOBEC-coupled | 5 hmC | Enzymatic treatment with |
| epigenetic sequencing | | T4-BGT and APOBEC3A |
| (ACE-seq) | | |

(Loise Williams, et al., Enzymatic Methyl-seq: The next generation of methylome analysis, New England Biolabs Expressions 2019. Feature Article.)

In preferred embodiments, methylation-specific reagents modify one nucleotide of the four typically-occurring nucleotides in a nucleic acid molecule (C, G, T, and A for DNA and C, G, U, and A for RNA), such that the reagent modifies the one nucleotide without modifying the other three nucleotides. The nucleotides resulting from conversion are not limited to the four typically occurring nucleotides listed above, and may include, for example, modified or variant forms of purine or pyrimidine structures, including, e.g., nucleobase analogs discussed herein. In preferred embodiments, the nucleotides produced by conversion are recognized by DNA modifying enzymes, e.g., DNA polymerases, as one of the typically occurring nucleotides listed above, and can serve as templates for strand replication. Conversion of nucleotides by any of the methods described herein may be detected by determining the sequence of a resulting strand, e.g., using standard sequencing methods, or by interrogating single or a few specific nucleotide locations to determine the identity of the nucleobase at the select locations.

As used herein, the term “converted” as used in reference to a nucleotide or DNA strand refers to a nucleotide or DNA strand that has been treated with a reagent or reagents under conditions in which some nucleotides are converted into other nucleotides. For example, in bisulfite conversion, cytosine bases in the DNA are typically deaminated, resulting in uracil bases at converted loci. While inefficient, bisulfite can also cause deamination of 5-methyl cytosine bases, resulting in thymine bases at converted loci. “Bisulfite-treated” and bisulfite-converted” are used interchangeably herein in reference to DNA or nucleotide loci that have been exposed to a bisulfite reagent under conditions in which unmethylated cytosine is typically converted to uracil. In the bisulfite-free TAPS process, methylated cytosines are selectively converted to dihydrouracil (DHU), while unmethylated Cs are not converted. The DHU nucleotides base pair with A nucleotides rather than G nucleotides, making them readily distinguishable from the unmethylated C bases in the converted DNA strands.

As used herein, the term “poorly converted,” as used in reference to conversion of a nucleotide upon treatment with reagent(s) and/or conditions under which some nucleotides are converted into other nucleotides, refers to a nucleotide having a reduced rate of conversion (preferably less than 10%, more preferably less than 1% of the rate of conversion) under the given treatment, as compared to the rate of conversion of a nucleotide expected to convert under the same treatment. For example, bisulfite-mediated deamination of cytosine is greatly slowed down by the presence of a 5-methyl group, such that the rate for the deamination of 5-methylcytosine to form thymine is about two orders of magnitude smaller than the rate for deamination of unmethylated cytosine to form uracil (see, e.g., Hayatsu et al., Biochemistry 18:4:632-37 (1979); Hayatsu, Proc. Jpn. Acad 84(8):321-330 (2008), each of which is incorporated herein by reference). Thus, 5-methylcytosine is said to be poorly converted compared to unmethylated cytosine under bisulfite treatment conditions typically used to convert unmethylated cytosine to uracil.

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715 and WO 2013/116375, each of which is incorporated by reference in its entirety). In some embodiments, bisulfite treatment is conducted in the presence of denaturing solvents such as but not limited to n-alkyleneglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8,-tetramethylchromane 2-carboxylic acid or trihydroxybenzone acid and derivatives thereof, e.g., Gallic acid (see: PCT/EP2004/011715, which is incorporated by reference in its entirety). In certain preferred embodiments, the bisulfite reaction comprises treatment with ammonium hydrogen sulfite, also referred to as ammonium bisulfite, e.g., as described in WO 2013/116375.

The term “methylation assay” refers to any assay for determining the methylation state of one or more CpG dinucleotide sequences within a sequence of a nucleic acid.

As used herein, the “sensitivity” of a given marker (or set of markers used together) refers to the percentage of samples that report a DNA methylation value above a threshold value that distinguishes between neoplastic and non-neoplastic samples. In some embodiments, a positive is defined as a histology-confirmed neoplasia that reports a DNA methylation value above a threshold value (e.g., the range associated with disease), and a false negative is defined as a histology-confirmed neoplasia that reports a DNA methylation value below the threshold value (e.g., the range associated with no disease). The value of sensitivity, therefore, reflects the probability that a DNA methylation measurement for a given marker obtained from a known diseased sample will be in the range of disease-associated measurements. As defined here, the clinical relevance of the calculated sensitivity value represents an estimation of the probability that a given marker would detect the presence of a clinical condition when applied to a subject with that condition.

As used herein, the “specificity” of a given marker (or set of markers used together) refers to the percentage of non-neoplastic samples that report a DNA methylation value below a threshold value that distinguishes between neoplastic and non-neoplastic samples. In some embodiments, a negative is defined as a histology-confirmed non-neoplastic sample that reports a DNA methylation value below the threshold value (e.g., the range associated with no disease) and a false positive is defined as a histology-confirmed non-neoplastic sample that reports a DNA methylation value above the threshold value (e.g., the range associated with disease). The value of specificity, therefore, reflects the probability that a DNA methylation measurement for a given marker obtained from a known non-neoplastic sample will be in the range of non-disease associated measurements. As defined here, the clinical relevance of the calculated specificity value represents an estimation of the probability that a given marker would detect the absence of a clinical condition when applied to a patient without that condition.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a nucleotide that is typically methylated and an unmethylated selected nucleotides refers specifically to a nucleotide that typically occurs in unmethylated form.

The term “methylation-specific restriction enzyme” refers to a restriction enzyme that selectively digests a nucleic acid dependent on the methylation state of its recognition site. In the case of a restriction enzyme that specifically cuts if the recognition site is not methylated or is hemi-methylated (a methylation-sensitive enzyme), the cut will not take place (or will take place with a significantly reduced efficiency) if the recognition site is methylated on one or both strands. In the case of a restriction enzyme that specifically cuts only if the recognition site is methylated (a methylation-dependent enzyme), the cut will not take place (or will take place with a significantly reduced efficiency) if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

The terms “selectively binds” and “specifically binds” (or selectively or specifically binding, bound, hybridizes, hybridizing, anneals, annealing, etc.) as used in reference to interaction between oligonucleotides or other nucleic acids are used herein interchangeably to refer to hybridization or base-pairing that is sufficiently sequence-selective that the oligonucleotide or nucleic acid will preferentially hybridize to a particular nucleic acid (e.g., a target nucleic acid having a particular nucleotide sequence) and will not substantially bind to non-target nucleic acids (e.g., having slightly or completely different nucleotide sequences than the target nucleic acid), under conditions in which selective or specific binding is conducted.

The term “primer” refers to an oligonucleotide, whether occurring naturally as, e.g., a nucleic acid fragment from a restriction digest, or produced synthetically, that is capable of acting as a point of initiation of synthesis when placed under conditions in which synthesis of a primer extension product that is complementary to a nucleic acid template strand is induced, (e.g., in the presence of nucleotides and an inducing agent such as a DNA polymerase, and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. Generally, the primer is sufficiently long to prime the synthesis of extension products in the presence of the inducing agent. The exact lengths of the primers will depend on many factors, including temperature, source of primer, and the use of the method.

The term “probe” refers to an oligonucleotide (e.g., a sequence of nucleotides), whether occurring naturally as in a purified restriction digest or produced synthetically, recombinantly, or by PCR amplification, that is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the present invention may, in some embodiments, be labeled with any “reporter molecule,” so that it is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “target,” as used herein refers to a nucleic acid sought to be sorted out from other nucleic acids, e.g., by probe binding, amplification, isolation, capture, etc. For example, when used in reference to the polymerase chain reaction, “target” refers to the region of nucleic acid bounded by the primers used for polymerase chain reaction, while when used in an assay in which target DNA is not amplified, e.g., in some embodiments of an invasive cleavage assay, a target comprises the site at which a probe and invasive oligonucleotides (e.g., INVADER oligonucleotide) bind to form an invasive cleavage structure, such that the presence of the target nucleic acid can be detected. A “segment” is defined as a region of nucleic acid within the target sequence. As used in reference to a double-stranded nucleic acid, the term “target” is not limited to a particular strand of the duplexed target, e.g., a coding strand, but may be used in reference to either one or both strands of, for example, a double-stranded gene or reference DNA.

Accordingly, as used herein, “non-target”, e.g., as it is used to describe a nucleic acid such as a DNA, refers to nucleic acid that may be present in a reaction, but that is not the subject of detection or characterization by the reaction. In some embodiments, non-target nucleic acid may refer to nucleic acid present in a sample that does not, e.g., contain a target sequence, while in some embodiments, non-target may refer to exogenous nucleic acid, i.e., nucleic acid that does not originate from a sample containing or suspected of containing a target nucleic acid, and that is added to a reaction, e.g., to normalize the activity of an enzyme (e.g., polymerase) to reduce variability in the performance of the enzyme in the reaction.

Nucleic acid may be isolated by any means, including the use of commercially available kits. Briefly, wherein the nucleic acid of interest is encapsulated by a cellular membrane, the biological sample can be disrupted and lysed by enzymatic, chemical or mechanical means. The nucleic acid solution may then be cleared of proteins and other contaminants, e.g., by digestion with proteinase K. The nucleic acid is then recovered from the solution. This may be carried out by means of a variety of methods including salting out, organic extraction, or binding of the nucleic acid to a solid phase support. The choice of method will be affected by several factors including time, expense, and required quantity of nucleic acid.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, be combined with other nucleic acids or molecules. For example, an isolated nucleic acid may be present in a host cell into which it has been placed, e.g., for heterologous expression.

The terms “purified” and “extracted” are used interchangeably herein and refer to molecules, either nucleic acids or polypeptides that are removed from their natural environment, isolated, or separated. An “isolated nucleic acid” may therefore be a purified nucleic acid. “Substantially purified” molecules are at least 60% free, preferably at least 75% free, and more preferably at least 90% free from other components with which they are naturally associated. As used herein, the terms “purified” or “to purify” also refer to the removal of contaminants from a sample.

As used herein, the terms “cell-free” and “circulating cell-free” as used in reference to nucleic acids from blood are used interchangeably and refer to nucleic acids, e.g., DNA and RNA species, that are found in blood but that are not within cells in the blood. The terms as used herein with respect to nucleic acid extracted from blood refer to the nature and location of the nucleic acid prior to collection of the sample from the subject and prior to extraction of the nucleic acid from the blood sample.

As used herein, the term “circulating tumor DNA” (or “ctDNA”) is tumor-derived DNA that is circulating in the peripheral blood of a patient. ctDNA is of tumor origin and originates directly from the tumor or from circulating tumor cells (CTCs), which are viable, intact tumor cells that shed from primary tumors and enter the bloodstream or lymphatic system.

The term “marker”, as used herein, refers to a substance (e.g., a nucleic acid, or a region of a nucleic acid, or a protein) that may be used to distinguish non-normal cells (e.g., cancer cells) from normal cells (non-cancerous cells), e.g., based on presence, absence, or status (e.g., methylation state) of the marker substance. As used herein “normal” methylation of a marker refers to a degree of methylation typically found in normal cells, e.g., in non-cancerous cells.

The term “neoplasm” as used herein refers to any new and abnormal growth of tissue, including but not limited to a cancer. Thus, a neoplasm can be a premalignant neoplasm or a malignant neoplasm.

The term “neoplasm-specific marker,” as used herein, refers to any biological material or element that can be used to indicate the presence of a neoplasm. Examples of biological materials include, without limitation, nucleic acids, polypeptides, carbohydrates, fatty acids, cellular components (e.g., cell membranes and mitochondria), and whole cells. In some instances, markers are particular nucleic acid regions (e.g., genes, intragenic regions, specific loci, etc.). Regions of nucleic acid that are markers may be referred to, e.g., as “marker genes,” “marker regions,” “marker sequences,” “marker loci,” etc.

The term “sample” is used in its broadest sense. In one sense it can refer to an animal cell or tissue or fluid. In another sense, it refers to a specimen or culture obtained from any source, as well as biological and environmental samples. Biological samples may be obtained from plants or animals (including humans) and encompass, e.g., fluids, solids, tissues, and gases. Human and animal samples include but are not limited to stool, tissue, sputum, mucus, blood or a blood product selected from plasma, serum, whole blood, an organ excretion such as pancreatic fluid, and urine. Environmental samples include environmental material such as surface matter, soil, water, and industrial samples. These examples are not to be construed as limiting the sample types applicable to the present invention. As used herein in reference to samples, the term “a sample” collected from a source or subject, e.g., from a patient, is not limited to a single physical specimen but also encompasses a sample that is collected in multiple portions, e.g., “a sample” of blood may be collected in two, three, four or more different blood collection tubes or other blood collection devices (e.g., bags), or combinations of different blood collection devices.

As used herein, the terms “suspected of comprising” or “suspected of containing” are used interchangeably to describe a feature that may or may not be present, e.g., a sample or subject, etc., that may or may not comprise or contain a particular feature, e.g., a marker nucleic acid, a target or a combination of targets (e.g., nucleic acids), or any other material or feature.

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present technology provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; pinnipeds; and horses. Thus, also provided is the diagnosis and treatment of livestock, including, but not limited to, domesticated swine, ruminants, ungulates, horses (including racehorses), and the like. The presently-disclosed subject matter further includes a system for diagnosing a cancer in a subject. The system can be provided, for example, as a commercial kit that can be used to screen for a risk of cancer or diagnose a cancer in a subject from whom a biological sample has been collected. An exemplary system provided in accordance with the present technology includes assessing the methylation state of a marker described herein.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), hot-start PCR (see, e.g., U.S. Pat. Nos. 5,773,258 and 5,338,671; each herein incorporated by reference in their entireties), intersequence-specific PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties). In some embodiments, a portion of a target nucleic acid is copied in the amplification, and in some embodiments, a non-target polynucleotide is amplified in response to the presence of a target nucleic acid, (e.g., a cleaved flap, ligation product, a rolling circle replication product, etc.)

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture of genomic or other DNA or RNA, without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (e.g., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (“PCR”). Because the desired amplified segments of the target sequence become the predominant sequences (in terms of concentration) in the mixture, they are said to be “PCR amplified” and are “PCR products” or “amplicons.” Those of skill in the art will understand the term “PCR” encompasses many variants of the originally described method using, e.g., real time PCR, nested PCR, reverse transcription PCR (RT-PCR), single primer and arbitrarily primed PCR, etc.

A “polymerase” is an enzyme generally for joining 3-OH 5-triphosphate nucleotides, oligomers, and their analogs. Polymerases include, but are not limited to, template-dependent DNA-dependent DNA polymerases, DNA-dependent RNA polymerases, RNA-dependent DNA polymerases, and RNA-dependent RNA polymerases. Polymerases include but are not limited to T7 DNA polymerase, T3 DNA polymerase, T4 DNA polymerase, T7 RNA polymerase, T3 RNA polymerase, SP6 RNA polymerase, DNA polymerase 1, Klenow fragment, Thermophilus aquaticus DNA polymerase, Tth DNA polymerase, Vent DNA polymerase (New England Biolabs), Deep Vent DNA polymerase (New England Biolabs), Bst DNA Polymerase Large Fragment, Stoeffel Fragment, 9° N DNA Polymerase, Pfu DNA Polymerase, Tfl DNA Polymerase, RepliPHI Phi29 Polymerase, Tli DNA polymerase, eukaryotic DNA polymerase beta, telomerase. Therminator polymerase (New England Biolabs), KOD HiFi DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting, and polymerases cited in US 2007/0048748, U.S. Pat. Nos. 6,329,178; 6,602,695; and U.S. Pat. No. 6,395,524 (incorporated by reference). These polymerases include wild-type, mutant isoforms, and genetically engineered variants.

A “DNA polymerase” is a polymerase that produces DNA from deoxynucleotide monomers (dNTPs). “Eubacterial DNA polymerase” as used herein refers to the Pol A type DNA polymerases (repair polymerases) from Eubacteria, including but not limited to DNA Polymerase I from E. coli, Taq DNA polymerase from Thermus aquaticus and DNA Pol I enzymes from other members of genus Thermus, and other eubacterial species etc.

As used herein, “preamplification reaction mixture” refers to a PCR amplification mixture for amplifying a particular target sequence in which the reaction mixture preferably does not contain reagents for directly detecting or measuring the amplified product, e.g., intercalating dyes or labeled probes such as FRET probes or FRET cassettes. In preferred embodiments, a preamplification reaction mixture is free of non-polymerase flap endonucleases (e.g., a eubacterial DNA polymerase comprising a 3′- or 5′-endo or exonuclease domain may be present but the preamplification mixture does not comprise a separate flap endonuclease, e.g., a FEN-1 endonuclease).

As used herein, “preamplifying” a target nucleic acid refers to amplifying the target nucleic acid to provide more copies of the target nucleic acids, e.g., prior to use of the amplified target material in a follow-own assay, e.g., a nucleic acid detection assay such as a PCR or PCR-flap assay, a sequencing assay, etc. In preferred embodiments, a target nucleic acid is preamplified in a preamplification reaction mixture, wherein the preamplification comprises thermal cycling the preamplification reaction mixture for fewer than 20 thermal cycles, preferably fewer than 19, 18, 17, 16, 15, 14, 13, 12, 11, 10, 9, 8, 7, 6, or 5 thermal cycles. Preferably, the number of thermal cycles is selected such that during the final thermal cycle, the amplification is in the exponential phase of the PCR.

As used herein, the term “primer annealing” refers to conditions that permit oligonucleotide primers to hybridize to template nucleic acid strands, preferably sufficiently to be extended by a DNA polymerase. Conditions for primer annealing vary with the length and sequence of the primer and are generally based upon the Tₘ that is determined or calculated for the primer. For example, an annealing step in an amplification method that involves thermocycling involves reducing the temperature after a heat denaturation step to a temperature based on the Tₘ of the primer sequence, for a time sufficient to permit such annealing.

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target.” In contrast, “background template” is used in reference to nucleic acid other than sample template that may or may not be present in a sample. The presence of background template is most often inadvertent. It may be the result of carryover, or it may be due to the presence of nucleic acid contaminants sought to be purified away from the sample. For example, nucleic acids from organisms other than those to be detected may be present as background in a test sample.

The term “next generation sequencing” or “NGS” refers to highly parallelized methods of performing nucleic acid sequencing and comprises the sequencing-by-synthesis or sequencing-by-ligation platforms (e.g., employed by Illumina, Life Technologies, Pacific Biosciences and Roche, etc.). Next generation sequencing methods may also include, but not be limited to, nanopore sequencing methods such as offered by Oxford Nanopore or electronic detection-based methods such as the Ion Torrent technology commercialized by Life Technologies. Nucleic acid sequencing techniques suitable for use with the present technology include, but are not limited to, sequencing by synthesis (see e.g., Meyer and Kircher, “Illumina sequencing library preparation for highly multiplexed target capture and sequencing,” Cold Spring Harbor Protocols 2010 (6)); single-molecule real-time sequencing (see e.g., Levene et al., “Zero-Mode Waveguides for Single-Molecule Analysis at High Concentrations,” Science. 299(5607): 682-6 (2003)); ion semiconductor sequencing (see e.g., Rusk, “Torrents of sequence,” Nat. Methods 8, 44 (2011)); pyrosequencing (see e.g., Wicker et al., “454 sequencing put to the test using the complex genome of barley,” BMC Genomics, 7:275, 2006); sequencing by ligation (SOLiD sequencing) (see e.g., Margulies et al., “Genome sequencing in microfabricated high-density picolitre reactors,” Nature, 437:376-80 (2005)); nanopore sequencing (see e.g., Goodwin et al., “Oxford Nanopore sequencing, hybrid error correction, and de novo assembly of a eukaryotic genome,” Genome Res., 25(11):1750-6 (2015)); chain termination sequencing (Sanger sequencing) (see e.g., Sanger et al., “DNA sequencing with chain-terminating inhibitors, “Proceedings of the National Academy of Sciences of the United States of America, 74 (12): 5463-5467 (1977)); and sequencing with mass spectrometry (see e.g., Edwards et al., “Mass-spectrometry DNA sequencing,” Mutation Research, 573(1-2): 3-12 (2005)).

As used herein, the term “nucleic acid detection assay” refers to any method of determining the presence or absence of, or amount of, or nucleotide composition of a nucleic acid of interest. Nucleic acid detection assays include but are not limited to, DNA sequencing methods including next generation sequencing methods, nucleic acid amplification methods, probe hybridization methods, structure specific cleavage assays (e.g., the INVADER assay, (Hologic, Inc.) and are described, e.g., in U.S. Pat. Nos. 5,846,717, 5,985,557, 5,994,069, 6,001,567, 6,090,543, and 6,872,816; Lyamichev et al., Nat. Biotech., 17:292 (1999), Hall et al., PNAS, USA, 97:8272 (2000), and U.S. Pat. No. 9,096,893, each of which is herein incorporated by reference in its entirety for all purposes); enzyme mismatch cleavage methods (e.g., Variagenics, U.S. Pat. Nos. 6,110,684, 5,958,692, 5,851,770, herein incorporated by reference in their entireties); polymerase chain reaction (PCR), described above; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884, 6,183,960 and 6,235,502, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (e.g., Baranay Proc. Natl. Acad. Sci USA 88, 189-93 (1991)); and sandwich hybridization methods (e.g., U.S. Pat. No. 5,288,609, herein incorporated by reference in its entirety).

In some embodiments, target nucleic acid is amplified (e.g., by PCR) and amplified nucleic acid is detected simultaneously using an invasive cleavage assay. Assays configured for performing a detection assay (e.g., invasive cleavage assay) in combination with an amplification assay are described in U.S. Pat. No. 9,096,893, incorporated herein by reference in its entirety for all purposes. Additional amplification plus invasive cleavage detection configurations, termed the QuARTS method, are described in, e.g., in U.S. Pat. Nos. 8,361,720; 8,715,937; 8,916,344; 9,212,392, and U.S. patent application Ser. No. 15/841,006 each of which is incorporated herein by reference for all purposes. The term “invasive cleavage structure” as used herein refers to a cleavage structure comprising i) a target nucleic acid, ii) an upstream nucleic acid (e.g., an invasive or “INVADER” oligonucleotide), and iii) a downstream nucleic acid (e.g., a probe), where the upstream and downstream nucleic acids anneal to contiguous regions of the target nucleic acid, and where an overlap forms between the 3′ portion of the upstream nucleic acid and duplex formed between the downstream nucleic acid and the target nucleic acid. An overlap occurs where one or more bases from the upstream and downstream nucleic acids occupy the same position with respect to a target nucleic acid base, whether or not the overlapping base(s) of the upstream nucleic acid are complementary with the target nucleic acid, and whether or not those bases are natural bases or non-natural bases. In some embodiments, the 3′ portion of the upstream nucleic acid that overlaps with the downstream duplex is a non-base chemical moiety such as an aromatic ring structure, e.g., as disclosed, for example, in U.S. Pat. No. 6,090,543, incorporated herein by reference in its entirety. In some embodiments, one or more of the nucleic acids may be attached to each other, e.g., through a covalent linkage such as nucleic acid stem-loop, or through a non-nucleic acid chemical linkage (e.g., a multi-carbon chain). As used herein, the term “flap endonuclease assay” includes “INVADER” invasive cleavage assays and QuARTS assays, as described above.

As used herein, the term “amplification reagents” refers to those reagents (deoxyribonucleoside triphosphates, buffer, etc.), needed for amplification except for primers, nucleic acid template, and the amplification enzyme. Typically, amplification reagents along with other reaction components are placed and contained in a reaction vessel.

A “reaction mixture” is a mixture of reagents (e.g., oligonucleotides, target nucleic acids, enzymes, etc.) in a combination and/or locus in which a reaction can occur, e.g., a mixture of reagents in a single reaction vessel, at a locus in a fluidic device, at locus on a surface, etc.

As used herein, a “multiplex” reaction is a reaction (e.g., PCR, PCR-flap assay) that operates on multiple targets (2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, etc.) in a single reaction mixture. Multiplexed reactions are distinguished from reactions that operate on one target analyte per reaction mixture. As used herein, the term “highly multiplexed” refers to reactions that operate on at least 6, preferably at least 10, more preferably at least 20 or more different targets (e.g., different genes, or regions of genes) in a single reaction mixture.

The terms “probe oligonucleotide,” “flap probe oligonucleotide” and “flap oligonucleotide” when used in reference to a flap assay, are used interchangeably and refer to an oligonucleotide that interacts with a target nucleic acid to form a cleavage structure in the presence of an invasive oligonucleotide.

As used herein, the term “label” refers to any moiety (e.g., chemical species) that can be detected or can lead to a detectable response. In some preferred embodiments, detection of a label provides quantifiable information. Labels can be any known detectable moiety, such as, for example, a sequence of nucleotides (e.g., a flap sequence), a radioactive label (e.g., radionuclides), a ligand (e.g., biotin or avidin), a chromophore (e.g., a dye or particle that imparts a detectable color), a hapten (e.g., digoxygenin), a mass label, latex beads, metal particles, a paramagnetic label, a luminescent compound (e.g., bioluminescent, phosphorescent or chemiluminescent labels) or a fluorescent compound (e.g., a compound that, upon excitation by radiation or light at one wavelength, emits energy, e.g., radiation or light, at a different wavelength). A label may be joined, directly or indirectly, to an oligonucleotide or other biological molecule. Direct labeling can occur through bonds or interactions that link the label to the oligonucleotide, including covalent bonds or non-covalent interactions such as hydrogen bonding, hydrophobic and ionic interactions, or through formation of chelates or coordination complexes. Indirect labeling can occur through use of a bridging moiety or “linker,” such as an antibody or additional oligonucleotide(s), which is/are either directly or indirectly labeled.

The term “invasive oligonucleotide” refers to an oligonucleotide that hybridizes to a target nucleic acid at a location adjacent to the region of hybridization between a probe and the target nucleic acid, wherein the 3′ end of the invasive oligonucleotide comprises a portion (e.g., a chemical moiety, or one or more nucleotides) that overlaps with the region of hybridization between the probe and target. The 3′ terminal nucleotide of the invasive oligonucleotide may or may not base pair with a nucleotide in the target. In some embodiments, the invasive oligonucleotide contains sequences at its 3′ end that are substantially the same as sequences located at the 5′ end of a portion of the probe oligonucleotide that anneals to the target strand. In some embodiments, e.g., in a PCR-flap assay, a primer used for amplification may also serve as an invasive oligonucleotide with probe.

As used herein in reference to cleavage of an invasive cleavage structure, the term “target cleavage site” refers to a preferred site (or sites) of cleavage on a nucleic acid structure (e.g., an invasive cleavage structure) by a structure-specific nuclease (e.g., a FEN-1 endonuclease) that recognizes the structure as a cleavage substrate. For example, as discussed by Kaiser, et al., 5′ flap endonucleases, including FEN-1 endonucleases, typically cleave an invasive cleavage structure in the downstream nucleic acid, generally after the first base-paired nucleotide, i.e., one nucleotide into the downstream duplex.

The term “flap endonuclease” or “FEN,” as used herein, refers to a class of nucleolytic enzymes, typically 5′ nucleases, that act as structure-specific endonucleases on DNA structures with a duplex containing a single stranded 5′ overhang, or flap, on one of the strands that is displaced by another strand of nucleic acid (e.g., such that there are overlapping nucleotides at the junction between the single and double-stranded DNA). FENs catalyze hydrolytic cleavage of the phosphodiester bond at the junction of single and double stranded DNA, releasing the overhang, or the flap. Flap endonucleases are reviewed by Ceska and Savers (Trends Biochem. Sci. 1998 23:331-336) and Liu, et al. (Annu. Rev. Biochem. 2004 73: 589-615; herein incorporated by reference in its entirety). FENs may be individual enzymes, multi-subunit enzymes, or may exist as an activity of another enzyme or protein complex (e.g., a DNA polymerase).

A flap endonuclease may be thermostable. For example, FEN-1 flap endonuclease from archival thermophiles organisms are typically thermostable. As used herein, the term “FEN-1” refers to a non-polymerase flap endonuclease from a eukaryote or archaeal organism. See, e.g., WO 02/070755, and U.S. Pat. No. 7,122,364, and Kaiser M. W., et al. (1999) J. Biol. Chem., 274:21387, which are all incorporated by reference herein in their entireties for all purposes.

As used herein, the term “cleaved flap” refers to a single-stranded oligonucleotide that is a cleavage product of a flap assay.

The term “cassette,” when used in reference to a flap cleavage reaction, refers to an oligonucleotide or combination of oligonucleotides configured to generate a detectable signal in response to cleavage of a flap or probe oligonucleotide, e.g., in a primary or first cleavage structure formed in a flap cleavage assay. In preferred embodiments, the cassette hybridizes to a non-target cleavage product produced by cleavage of a flap oligonucleotide to form a second overlapping cleavage structure, such that the cassette can then be cleaved by the same enzyme, e.g., a FEN-1 endonuclease.

In some embodiments, the cassette is a single oligonucleotide comprising a hairpin portion (i.e., a region wherein one portion of the cassette oligonucleotide hybridizes to a second portion of the same oligonucleotide under reaction conditions, to form a duplex). In other embodiments, a cassette comprises at least two oligonucleotides comprising complementary portions that can form a duplex under reaction conditions. In preferred embodiments, the cassette comprises a label, e.g., a fluorophore. In particularly preferred embodiments, a cassette comprises labeled moieties that produce a FRET effect.

As used herein, the term “FRET” refers to fluorescence resonance energy transfer, a process in which moieties (e.g., fluorophores) transfer energy e.g., among themselves, or, from a fluorophore to a non-fluorophore (e.g., a quencher molecule). In some circumstances, FRET involves an excited donor fluorophore transferring energy to a lower-energy acceptor fluorophore via a short-range (e.g., about 10 nm or less) dipole-dipole interaction. In other circumstances, FRET involves a loss of fluorescence energy from a donor and an increase in fluorescence in an acceptor fluorophore. In still other forms of FRET, energy can be exchanged from an excited donor fluorophore to a non-fluorescing molecule (e.g., a “dark” quenching molecule, e.g., “BHQ” quenchers, Biosearch Technologies). FRET is known to those of skill in the art and has been described (See, e.g., Stryer et al., 1978, Ann. Rev. Biochem., 47:819; Selvin, 1995, Methods Enzymol., 246:300; Orpana, 2004 Biomol Eng 21, 45-50; Olivier, 2005 Mutant Res 573, 103-110, each of which is incorporated herein by reference in its entirety).

As used herein, the term “FRET system” refers to a pair or group of moieties that together act as donor-acceptor or donor-quencher partners for FRET-based analysis of molecules, e.g., probe oligonucleotides, flap oligonucleotides, or other assay reporter molecules. While embodiments of the technology are illustrated with a fluorophore in one particular position and a quencher or other FRET acceptor moiety in a particular second position. For example, in some PCR probe oligonucleotides, e.g., for TAQMAN assays, the fluorophore is at or near one end of a probe oligonucleotide and the quencher moiety is at or near the other end of the probe oligonucleotide.

Suitable fluorophores include but are not limited to fluorescein, rhodamine, REDMOND RED dye, YAKIMA YELLOW dye, hexachloro-fluorescein, TAMRA dye, ROX dye, Cy3, Cy3.5, Cy5, Cy5.5, and Cy7, Quasar 570 (Q570), Quasar 670 (Q670), Quasar 705 (Q705) (Quasar dyes from LGC, Biosearch Technologies), 4,4-difluoro-5,7-diphenyl-4-bora-3a,4a-diaza- -s-indacene-3-propionic acid, 4,4-difluoro-5,p-methoxyphenyl-4-bora-3a,4a-- diaza-s-indacene-3-propionic acid, 4,4-difluoro-5-styryl-4-bora-3a,4-adiaz- a-S-indacene-propionic acid, 6-carboxy-X-rhodamine, N,N,N′,N′-tetramethyl-6-carboxyrhodamine, Texas Red, eosin, fluorescein, 4,4-difluoro-5,7-diphenyl-4-bora-3a,4a-diaza-s-indacene-3-propionic acid, 4,4-difluoro-5,p-ethoxyphenyl-4-bora-3a,4a-diaza-s-indacene 3-propionic acid and 4,4-difluoro-5-styryl-4-bora-3a,4a-diaza-S-indacene-propionic acid, 6-carboxyfluorescein (6-FAM), 2′,4′,1,4,-tetrachlorofluorescein (TET), 21,4′,51,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), 2′-chloro-7′-phenyl-1,4-dichloro-6-carboxyfluorescein (VIC), fluorescein isothiocyanate (FITC), 5,6-carboxymethyl fluorescein, Texas red, nitrobenz-2-oxa-1,3-diazol-4-yl (NBD), coumarin, dansyl chloride, amino-methyl coumarin (AMCA), Erythrosin, BODIPY dye, CASCADE BLUE dye, OREGON GREEN dye, pyrene, lissamine, xanthenes, acridines, oxazines, phycoerythrin, QUANTUM DYE, thiazole orange-ethidium heterodimer, and the like.

Suitable quenchers include, but are not limited to, cyanine dyes, e.g., Cy3, Cy3.5, Cy5, Cy5.5, and Cy7, rhodamine dyes, e.g., tetramethyl-6-carboxyrhodamine (TAMRA) and tetrapropano-6-carboxyrhodamine (ROX), DABSYL dye, DABCYL dye, cyanine dyes, nitrothiazole blue (NTB), anthraquinone, malachite green, nitrothiazole, or nitroimidazole compounds, QSY7 (Molecular Probes, Eugene, OR), ECLIPSE quencher (Nanogen, San Diego, CA), and the like. Analysis of factors such as absorbance and emission spectra of various molecules in selection of pairs or groups of moieties for use in FRET configurations is well known to those of skill in the art.

In an exemplary flap detection assay, an invasive oligonucleotide and flap oligonucleotide are hybridized to a target nucleic acid to produce a first complex having an overlap as described above. An unpaired “flap” is included on the 5′ end of the flap oligonucleotide. The first complex is a substrate for a flap endonuclease, e.g., a FEN-1 endonuclease, which cleaves the flap oligonucleotide to release the 5′ flap portion. In a secondary reaction, the released 5′ flap product serves as an invasive oligonucleotide on a FRET cassette to again create the structure recognized by the flap endonuclease, such that the FRET cassette is cleaved. When the fluorophore and the quencher are separated by cleavage of the FRET cassette, a detectable fluorescent signal above background fluorescence is produced.

As used herein, the term “PCR-flap assay” refers to an assay configuration combining PCR target amplification and detection of the amplified DNA by formation of an overlap cleavage structure comprising amplified target DNA, and, in preferred embodiments, formation of a second overlap cleavage structure comprising a cleaved 5′ flap from the first overlap cleavage structure and a labeled reporter oligonucleotide, e.g., a “FRET cassette” or 5′ hairpin FRET reporter oligonucleotide. In preferred embodiments of the PCR-flap assay as used herein, the assay reagents comprise a mixture containing DNA polymerase, FEN-1 endonuclease, a primary probe comprising a portion complementary to a target nucleic acid. Optionally, PCR-flap assay reagents further comprise a FRET cassette or 5′ hairpin FRET reporter. Typically, target nucleic acid is amplified by PCR and the amplified nucleic acid is detected simultaneously (i.e., detection occurs during the course of target amplification). PCR-flap assays include the QuARTS assays described in U.S. Pat. Nos. 8,361,720; 8,715,937; and 8,916,344; flap assay using probe oligonucleotides having a longer target-specific region (Long probe Quantitative Amplified Signal, “LQAS”) is described in U.S. Pat. No. 10,648,025; and the amplification assays of U.S. Pat. No. 9,096,893 (for example, as diagrammed in FIG. 1 of that patent), each of which is incorporated herein by reference in its entirely.

As used herein, the term “PCR-flap assay reagents” refers to one or more reagents for detecting target sequences in a PCR-flap assay, the reagents comprising nucleic acid molecules capable of participating in amplification of a target nucleic acid and in formation of a flap cleavage structure in the presence of the target sequence, in a mixture containing DNA polymerase, primers, FEN-1 endonuclease, and a probe or flap oligonucleotide, optionally a reverse transcriptase. PCR-flap assay reagents may further comprise a FRET cassette or 5′ hairpin FRET reporter.

As used herein, a “5′ hairpin FRET reporter” or “5′ hairpin FRET cassette” refers to a type of FRET probe or FRET cassette that comprises a 5′ hairpin-forming region upstream of a target cleavage site, and that comprises FRET labeling moieties that are separated upon cleavage of the reporter at the target cleavage site (see, e.g., WO 2021/055508, which is incorporated herein by reference in its entirety).

As used herein, the term “amplification bias” refers to differences in amplification efficiencies, i.e., the number of copies produced from a target nucleic acid, between and among different target nucleic acid sequences, when treated under the same amplification reaction conditions.

The term “low-bias amplification buffer” as used herein refers to an amplification buffer composed to exhibit low target-to-target variation in amplification efficiency for different targets amplified together in a multiplexed amplification reaction, e.g., a multiplexed preamplification reaction prior to a follow-on amplification reaction, e.g., a PCR-flap assay reaction. Amplification bias between different targets can be assessed by measuring the efficiencies of amplification for different targets of known concentration. In some embodiments of the present technology, low-bias amplification buffer is a buffer useful for PCR-flap assays and comprising a high magnesium concentration (e.g., at least 6 mM, preferably 6 to 10 mM, preferably, 7 to 9 mM, preferably at about 7.5 mM as a final concentration in a reaction mixture), in contrast to PCR buffers that typically comprise about 1 to 4 mM final concentration of magnesium in a reaction mixture. In preferred embodiments, a low-bias amplification buffer comprises 3-(n-morpholino) propanesulfonic acid (MOPS) buffer, and in certain preferred embodiments, a low-bias amplification buffer comprises 7.5 mM MgCl₂, 10 mM MOPS, 0.3 mM Tris-HCl, pH 8.0, 0.8 mM KCl, 0.1 μg/μL BSA, 0.0001% Tween-20, and 0.0001% IGEPAL CA-630. Herein the terms “PCR-flap assay buffer” and “low-bias amplification buffer” are used interchangeably.

Ranges for the amount of Mg⁺⁺ used in a low-bias amplification buffer include any concentration encompassed by the ranges discussed above. For example, a low-bias amplification buffer may contain 6, 6.1, 6.2, 6.5, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0 mM Mg⁺⁺, or more, or any fractional value concentration therebetween. These concentrations are recited by way of example and not of limitation.

As used herein, “low-bias amplification buffer” is distinct from PCR buffers that comprise Mg⁺⁺ above about 4 mM, but that further comprise reagents that reduce the helix-stabilizing effect of a high Mg⁺⁺ concentration, e.g., DMSO, (NH₄)₂SO₄, betaine, etc., sometimes referred to as enhancer reagents. For discussion of such enhancer reagents, see, e.g., Ralser, et al., An efficient and economic enhancer mix for PCR, Biochemical and Biophysical Research Communications 347 (2006) 747-751; O. Henegariu, et al., “Multiplex PCR: Critical Parameters and Step-by-Step Protocol,” BioTechniques 23:504-511 (September 1997); and Qiagen PCR Brochure “Maximizing PCR and RT-PCR Success,” Third Edition (Document 1104683, 10/2016), each of which is incorporated herein by reference in its entirety. In preferred embodiments, low-bias amplification buffer is essentially free of (NH₄)₂SO₄. In some embodiments, a low-bias amplification buffer is essentially free of ammonium ions. In some embodiments, low-bias amplification buffer is essentially free of DMSO and/or betaine.

As used herein, the term “essentially free of” as used in reference to a component excluded from a composition, e.g., a reaction mixture, a buffer, etc., refers to a composition that is not formulated to include the excluded component, but that may comprise trace amounts of the component, e.g., by carry-over from an earlier reaction step, or from a concentrated stock of a reaction component that is added in a small amount, e.g., an enzyme in a storage solution. For example, a small amount of glycerol, or other material may be introduced into a reaction mixture (e.g., a PCR flap assay reaction mixture) by addition of an aliquot of an enzyme (e.g., a polymerase) that includes the material for stable storage, but such materials are typically diluted sufficiently in the reaction mixture that the trace amounts of glycerol and other material do not alter the expected function of the reaction mixture, e.g., a trace amount of (NH₄)₂SO₄, betaine, or DMSO, would not be expected to alter the low-bias multiplex amplification results observed when a low-bias amplification buffer described herein is used. Such trace amounts of materials are typically not reported in literature as part of the formulation of the final reaction mixture. Thus, a composition that is “essentially free” of a recited component may contain the excluded component at a level such that, though present, it does not alter the function of the recited composition as compared to a pure composition, i.e., a composition that is completely free of the excluded component.

As used herein, the term “essentially the same amount” and “essentially the same concentration,” as used e.g., in reference to components of a mixture including but not limited to primers, probes or other oligonucleotides present in an preamplification reaction mixture, refers to amounts of reagents that are formulated to have the same concentrations in the mixture, but that may comprise minor variations in relative amounts or numbers of copies of the different components, e.g., due to carry over of some amount of one or more of the components from an earlier reaction step, or through variations in the normal course of laboratory preparation or measurement. Thus, a composition that is recited to contain “essentially equal amounts” of different components, or that contains components at “essentially the same concentration” may contain the components in amounts that vary slightly, but such that, though there may be differences in absolute amounts or concentrations, the differences do not alter the function of the recited composition as compared to a composition in which the amounts of the different components are at precisely equal amounts or concentrations. The terms “essentially the same amount” and “essentially the same concentration” are used interchangeably in reference to both liquid preparations and dried preparations. As used in reference to primers, probes or other oligonucleotides, the terms generally refer to molar amounts of the molecules, or molar amounts of specific parts of molecules (e.g., molar amounts of extendible 3′ ends of primers).

The term “real time” as used herein in reference to detection of nucleic acid amplification or signal amplification refers to the detection or measurement of the accumulation of products or signal in the reaction while the reaction is in progress, e.g., during incubation or thermal cycling. Such detection or measurement may occur continuously, or it may occur at a plurality of discrete points during the progress of the amplification reaction, or it may be a combination. For example, in a polymerase chain reaction, detection (e.g., of fluorescence) may occur continuously during all or part of thermal cycling, or it may occur transiently, at one or more points during one or more cycles. In some embodiments, real time detection of PCR or PCR-flap assay reactions is accomplished by determining a level of fluorescence at the same point (e.g., a time point in the cycle, or temperature step in the cycle) in each of a plurality of cycles, or in every cycle. Real time detection of amplification may also be referred to as detection “during” the amplification reaction.

As used herein, the terms “reverse transcription” and “reverse transcribe” refer to the use of a template-dependent polymerase to produce a DNA strand complementary to an RNA template. A polymerase capable of producing a DNA strand complementary to an RNA template is generally referred to as a “reverse transcriptase” or as a polymerase that has “reverse transcriptase activity”.

As used herein, the term “abundance of nucleic acid” refers to the amount of a particular target nucleic acid sequence present in a sample or aliquot. The amount is generally referred to in terms of mass (e.g., μg), mass per unit of volume (e.g, μg/μL); copy number (e.g., 1000 copies, 1 attomole), or copy number per unit of volume (e.g, 1000 copies per mL, 1 attomole per μL). Abundance of a nucleic acid can also be expressed as an amount relative to the amount of a standard of known concentration or copy number. Measurement of abundance of a nucleic acid may be on any basis understood by those of skill in the art as being a suitable quantitative representation of nucleic acid abundance, including physical density or the sample, optical density, refractive property, staining properties, or on the basis of the intensity of a detectable label, e.g., a fluorescent label.

The term “amplicon” or “amplified product” refers to a segment of nucleic acid, generally DNA, generated by an amplification process such as the PCR process or another replication process, e.g., rolling circle or LAMP amplification processes. The terms are also used in reference to RNA segments produced by amplification methods that employ RNA polymerases, such as NASBA, TMA, etc.

As used herein, “rolling circle amplification” refers to in vitro rolling circle replication of a circular nucleic acid using a strand-displacing DNA polymerase to form a DNA molecule comprising tandem repeats of a sequence complementary to the circular nucleic acid, as described, e.g., in U.S. Pat. Nos. 6,210,884; 6,183,960; 6,235,502; 5,942,391; 6,316,229; 7,862,999; 11,186,863; U.S. Pat. Publication US 2015/0284786; and in M. Ali, et al. “Rolling circle amplification: a versatile tool for chemical biology, materials science and medicine”. Chemical Society Reviews. 43 (10): 3324-3341.

The term “amplification plot” as used in reference to a thermal cycling amplification reaction refers to the plot of signal that is indicative of amplification, e.g., fluorescence signal, versus cycle number. When used in reference to a non-thermal cycling amplification method, an amplification plot generally refers to a plot of the accumulation of signal as a function of time.

The term “baseline” as used in reference to an amplification plot refers to the detected signal coming from assembled amplification reactions prior to incubation or, in the case of PCR, in the initial cycles, in which there is little change in signal.

The term “no template control” and “no target control” (or “NTC”) as used herein in reference to a control reaction refers to a reaction or sample that does not contain template or target nucleic acid. It is used to verify amplification quality.

As used herein, the term “quantitative amplification data set” refers to the data obtained during quantitative amplification of the target sample, e.g., target DNA. In the case of quantitative PCR or QuARTS assays, the quantitative amplification data set is a collection of fluorescence values obtained at during amplification, e.g., during a plurality of, or all of the thermal cycles. Data for quantitative amplification is not limited to data collected at any particular point in a reaction, and fluorescence may be measured at a discrete point in each cycle or continuously throughout each cycle.

The term “Ct” or “threshold cycle” as used herein in reference to real time detection during an amplification reaction that is thermal cycled refers to the fractional cycle number at which the detected signal (e.g., fluorescence) passes the fixed threshold.

The abbreviations “Ct” and “Cp” or “threshold cycle” as used herein in reference to data collected during an amplification reaction, e.g., real time PCR and PCR-flap assays refer to the cycle at which signal (e.g., fluorescent signal) crosses a predetermined threshold value indicative of positive signal. Various methods have been used to calculate the threshold that is used as a determinant of signal versus concentration, and the value is generally expressed as either the “crossing threshold” (Ct) or the “crossing point” (Cp). Either Cp values or Ct values may be used in embodiments of the methods presented herein for analysis of real-time signal for the determination of the percentage of variant and/or non-variant constituents in an assay or sample.

As used herein the term “fish DNA” refers to bulk (e.g., genomic) DNA isolated from fish, e.g., as described in U.S. Pat. No. 9,212,392. Bulk purified fish DNA is commercially available, e.g., provided in the form of cod and/or herring sperm DNA (Roche Applied Science, Mannheim, Germany) or salmon DNA (USB/Affymetrix).

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides.

The term “system” as used herein refers to a collection of articles for use for a particular purpose. In some embodiments, the articles comprise instructions for use, as information supplied on e.g., an article, on paper, or on recordable media (e.g., DVD, CD, flash drive, etc.). In some embodiments, instructions direct a user to an online location, e.g., a website.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, percentage methylation, allele frequencies, RNA expression levels, protein expression, phenotypes correlating to genotypes, etc. “Allele frequency information” refers to facts or data pertaining to allele frequencies, including, but not limited to, allele identities, statistical correlations between the presence of an allele and a characteristic of a subject (e.g., a human subject), the presence or absence of an allele in an individual or population, the percentage likelihood of an allele being present in an individual having one or more particular characteristics, etc.

### BRIEF DESCRIPTION OF THE DRAWINGS

FIG. 1 provides a schematic diagram of assays in which different target nucleic acids (e.g., different genes and gene variants, cDNAs, single nucleotide polymorphisms) are preamplified together in a multiplex reaction, then different targets are detected using the same reporter dye by conducting PCR-flap assays in separate reaction mixtures.

FIG. 2 provides a schematic diagram of assays in which different target nucleic acids are preamplified together in a multiplex reaction, then two different targets and a reference target are detected using two different reporter dyes normalized to a third reference dye in a triplex PCR-flap assay reaction mixture.

FIG. 3 provides tables showing an arrangement of 3-dye triplexed PCR-flap assays as illustrated in FIG. 2, as they may be used to detect 12 different targets, e.g., marker DNAs preamplified from a DNA sample.

FIG. 4 provides a schematic diagram of an embodiment of the present technology, in which different target nucleic acids are preamplified together in a multiplex reaction under bias-minimizing conditions, then the aggregate signal from multiple different targets is measured using a single dye, e.g., using the same FRET cassette. In some embodiments, an additional set of markers report to a FRET cassette with a second dye, and additional sets of markers report to third, fourth, fifth, etc., dyes.

FIG. 5A provides tables showing an arrangement of 2 multiplexed PCR-flap assays as illustrated in FIG. 4, as they may be used to detect 12 different targets, e.g., marker DNAs preamplified from a DNA sample along with a reference gene, using 1 dye for all 6 markers and 1 dye for the reference gene.

FIG. 5B provides tables showing an arrangement of a single multiplexed PCR-flap assays as illustrated in FIG. 4, as it may be used to detect 12 different targets, e.g., marker DNAs preamplified from a DNA sample along with a reference gene, using 2 dyes to detect each of two different sets of 6 markers and a third dye for the reference gene.

FIG. 5C provides tables showing an arrangement of a single multiplexed PCR-flap assays as illustrated in FIG. 4, as it may be used to detect 24 different targets, e.g., marker DNAs preamplified from a DNA sample along with a reference gene, using 2 dyes to detect each of two different sets of 12 markers and a third dye for the reference gene.

FIG. 6 shows results of varying MgCl₂ concentration in multiplex preamplification in a PCR-flap assay buffer on the number of strands measured for each of the indicated marker DNAs in follow-on LQAS PCR-flap assay reactions. These data show the marker-to-marker variability in the follow-on LQAS PCR-flap assay reactions when either 100 or 1000 strands of marker DNA was provided in the multiplex preamplification reaction. The calculated theoretical yield of amplified strands (81920 for 1000 strands of input target DNA material, or 8192 for 100 strands of input target DNA) is shown as a dashed horizontal line on each graph.

FIG. 7 shows results of conducting multiplex preamplification in the low-bias high Mg⁺⁺ PCR-flap assay buffer (7.5 mM MgCl₂) compared to preamplification of the same combination of target DNAs in an (NH₄)₂SO₄ PCR buffer having 6.7 mM MgCl2 in combination with additional helix-destabilizing components (e.g., dimethyl sulfoxide) of varying MgCl₂ concentration in multiplex preamplification in a PCR-flap assay buffer on the number of strands measured for each of the indicated marker DNAs in follow-on LQAS PCR-flap assay reactions. These data show the marker-to-marker variability in the follow-on LQAS PCR-flap assay reactions when either 100 or 1000 strands of marker DNA was provided in the multiplex preamplification reaction. The calculated theoretical yield of amplified strands (81920 for 1000 strands of input target DNA material, or 8192 for 100 strands of input target DNA) is shown as a dashed horizontal line on each graph.

FIG. 8 provides a table of three-dye triplex PCR-flap assay combinations for detecting the indicated marker DNAs in combinations. The “triplex name” is composed of the first letters of each marker detected in the triplex reactions.

FIGS. 9A-9G show exemplary oligonucleotide combinations (primers, probes and FRET cassette oligonucleotides) as used in combination with the indicated concentrations of dNTPs in triplex PCR-flap assays.

FIG. 10 provides exemplary combinations for multiplexed PCR-flap assays in which multiple markers generate signal using each of the dyes indicated, such that the listed markers are assayed using only four highly-multiplexed PCR-flap assays.

FIGS. 11A-11D show exemplary oligonucleotide combinations (primers, probes, and FRET cassette oligonucleotides) as used in combination with the indicated concentrations of dNTPs in the four highly-multiplexed PCR-flap assays shown in table in FIG. 10.

FIG. 12 shows a table of results from assaying the samples from individuals having cancer and healthy individuals using low-bias multiplex preamplification followed by four MAD-PCR-flap assay reactions using the combinations of oligonucleotides shown in FIG. 10.

FIGS. 13A-13D shows tables of results from assaying the samples from individuals having cancer and healthy individuals using low-bias multiplex preamplification followed by triplex PCR-flap assay reactions using the combinations of oligonucleotides shown in FIGS. 9A-9G. The percent methylation of each marker measured in the triplex reactions is compared to the combined percent methylation measured for those markers in the corresponding MAD reaction (MAD reaction 1, 2, 3, or 4, as indicated).

### DETAILED DESCRIPTION OF THE INVENTION

The technology relates to multiplexed preamplification of nucleic acids under conditions that reduce bias between the amounts of amplified product produced from different target nucleic acid sequences coamplified in a preamplification reaction mixture. For example, different target nucleic acids may amplify with different efficiencies, such that different numbers of copies are produced, even if the same number of copies of each target nucleic acid are present prior to amplification. Differences in amplification efficiencies between and among different target nucleic acid sequences under the same reaction conditions may be referred to as “amplification bias”. Provided herein are technologies relating to low-bias amplification buffers and use of low-bias amplification buffers in multiplexed preamplifcation of nucleic acids, wherein the preamplified products are further assayed in multiplexed nucleic acid detection assays, e.g., PCR or PCR-flap assays, nucleic acids sequencing assays, etc. Exemplary, non-limiting methods are described below.

In particular embodiments, the multiplex preamplified sample is further analyzed in a multiplexed nucleic acid detection assay in which two or more different target nucleic acids produce signal using the same label, e.g., the same fluorophore, such that the total signal from that label comprises signal generated by detection of multiple different nucleic acids or nucleic acid sequences, in an additive effect. Generally, when assays are configured to produce additive signals from multiple different targets, the components of the amplification mixture, e.g., the individual primer concentrations for each target, are adjusted so that the amplifications from different target nucleic acids in a multiplexed reaction have similar amplification efficiencies. See, e.g., (Sint, D., et al., Methods in Ecology and Evolution 2012, 3, 898-905, and WO 2006/050499A2). In developing the present technology, it has surprisingly been found that use of a low-bias amplification buffer during multiplexed preamplification avoids the need to use different concentrations of different primers in either the multiplexed preamplification or in the follow-on multiplexed nucleic acids detection assay.

Provided herein are technologies relating to methods of characterizing a sample or combination of samples from a subject comprising analyzing the sample(s) for amounts of different nucleic acids, e.g., different alleles, mutations, single nucleotide polymorphisms (SNPs),methylation markers, different regions of genes or chromosomes, or characteristic nucleic acids from different species or variants, e.g., in a sample comprising nucleic acids from multiple organisms or different cell types.

Provided herein is technology relating to reducing bias in amplification-based detection of nucleic acids and particularly, but not exclusively, to methods for enriching low-nucleic acid samples for analysis. In some embodiments, the nucleic acid is pretreated, e.g., DNA is pretreated with a methylation-sensitive reagent or enzyme, or RNA is reverse transcribed.

Biological samples of interest may have vastly different amounts of nucleic acid in them and even, if rich in bulk nucleic acid, may have very low amounts of particular nucleic acids of interest, e.g., non-normal DNAs or RNAs within a background of normal DNA or RNA, or human nucleic acid in a background of microbial or viral nucleic acid (or vice versa). To compensate for a low concentration of target nucleic acids, a large sample may sometimes be processed to collect sufficient nucleic acid for a particular assay. However, when it is desirable to subject a sample with a low concentration of target nucleic acids to a number of different assays in parallel, the necessary sample size may become prohibitively large. For example, circulating cell-free DNA in plasma (cfDNA), e.g., of a subject, is typically very low, as it is continuously cleared from the bloodstream, mainly by the liver, and has a half-life of only 10 to 15 minutes. The typical levels of circulating DNA are thus very low, e.g., for healthy individuals, a particular segment of DNA, e.g., from a gene of interest, may be present at about 1,500-2000 copies/mL, while a segment of DNA associated with a tumor may be present at about 5000 copies/mL in a subject with a late-stage cancer. Further, tumor-derived cfDNA in plasma is typically fragmented into short strands, e.g., of 200 or fewer base pairs (see, e.g., P. Jiang, et al., Proc. Natl Acad Sci. 112(11): E1317-E1325 (2015), incorporated herein by reference in its entirety). Fetal-derived cfDNA in maternal blood is not only small in size, it is also present as a minor fraction of the total cfDNA circulating in blood of a pregnant subject. Such small DNAs are especially hard to purify because they can be lost during typical purification steps, e.g., through inefficiencies in precipitation and/or DNA binding purification steps.

Recovery of the cfDNA from such blood fraction samples may capture 75%, but often much less is recovered. Similarly, viruses and/or their nucleic acids may be present in a sample from an infected individual at low concentration, e.g., per mL of blood or plasma. Thus, depending on the sensitivity of the particular assay for these target nucleic acids, analysis of multiple nucleic acid sequences from plasma can require large amounts of plasma from a subject. Enrichment by targeted preamplification of specific target regions can increase the number of markers that can be analyzed using the same starting sample, i.e., without the need to collect correspondingly larger samples (e.g., plasma or blood) from the subject.

The technology is further particularly suited for multiplex analysis of any sample in which individual species of target nucleic acid may be minor fractions of a total preparation of nucleic acid from the sample. For example, samples such as environmental samples may comprise a complex mixture of nucleic acids, e.g., from eukaryotic cells, bacterial cells, archaeal cells, and/or from different bacterial, fungal, archaeal and/or viral species, or mutants or variants thereof. Multiplex preamplification can increase the number of genes, species, variants, etc., that can be characterized in a single sample, e.g., a soil or water sample, facilitating, for example, microbiome analysis or detection of emergent new variants.

Provided herein are embodiments of technologies for using low-bias multiplexed preamplification particularly suited for analysis of target nucleic acids that are in low abundance and/or that are fragmented in the samples in which they are found.

In some embodiments, target nucleic acids have been treated with a methylation-sensitive conversion reagent, e.g., a bisulfite reagent or using the TAPS method combining oxidation by TET enzymes with reduction by borane derivatives, as described herein.

### EMBODIMENTS OF THE TECHNOLOGY

### Low Bias Preamplification of Target Regions

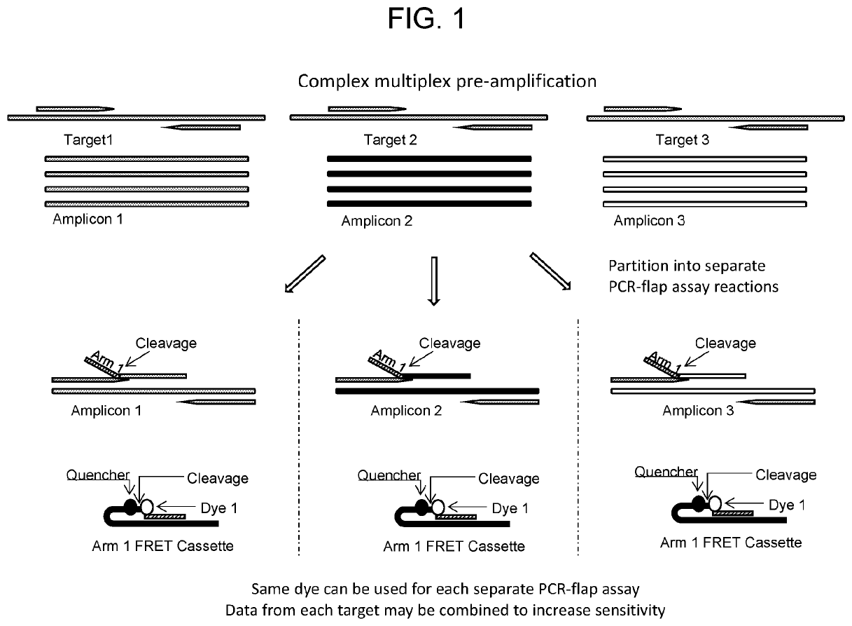

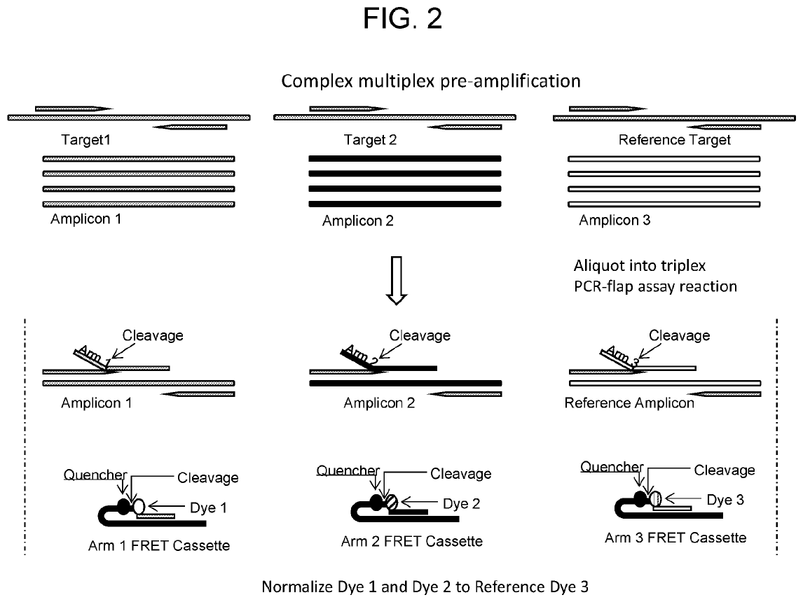

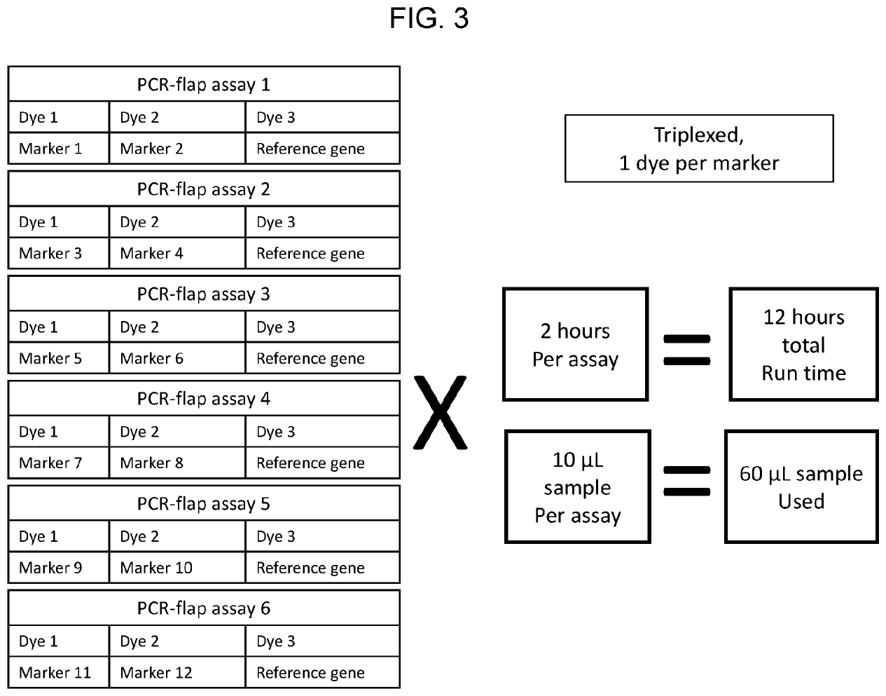

Provided herein is technology related to providing an increased amount of DNA for analysis in a follow-on nucleic acid detection assay, in particular a PCR assay such as a PCR-flap assay, e.g., a QuARTS or LQAS assay as diagramed in FIG. 1. In particular, embodiments of the methods and compositions disclosed herein provide for increasing an amount of multiple different nucleic acids targets of interest, e.g., from a low-target sample, using a low-bias multiplexed preamplification step, followed by one or more detection assays, e.g., PCR-flap assays. In some embodiments, a target nucleic acid is RNA, and preamplification comprises reverse transcription.

In preferred embodiments, the methods are conducted in reaction mixtures that comprise a low-bias amplification buffer, e.g., a PCR-flap assay buffer having high Mg⁺⁺ and low KCl relative to standard PCR buffers, (e.g, 6-10 mM, preferably 7.5 mM Mg⁺⁺, and 0.0 to 0.8 mM KCl). For example, a typical PCR buffer (final reaction concentration, or “1×”) is 1.5 mM MgCl₂, 20 mM Tris-HCl, pH 8, and 50 mM KCl, while an exemplary 1×PCR-flap assay buffer comprises 7.5 mM MgCl₂, 10 mM MOPS, 0.3 mM Tris-HCl, pH 8.0, 0.1 μg/μL BSA, 0.0001% Tween-20, and 0.0001% IGEPAL CA-630. KCl may be included, e.g., at final concentration of 0.8 mM, 25 mM, or at any other concentration that does not reduce the low-bias effect of the buffer when used in a multiplex amplification reaction. Preferably, the low-bias amplification buffer is essentially free of (NH₄)₂SO₄. In particularly preferred embodiments, the low-bias amplification buffer is essentially free of DMSO, formamide and added reducing agents, such as DTT and β-mercaptoethanol.

PCR-flap assays use different buffer and salt conditions than standard PCR (e.g., a PCR-flap assay buffer typically comprises MOPS, Tris-HCl pH 8.0, and 7.5 mM MgCl₂, and little or no added KCl or other monovalent salt, conditions typically considered unfavorable for PCR due to the low monovalent salt and the relatively high concentration of Mg¹¹ (see, e.g., “Guidelines for PCR Optimization with Taq DNA Polymerase” https://www.neb.com/tools-and-resources/usage-guidelines/guidelines-for-pcr-optimization-with-taq-dna-polymerase, which discloses 1.5 mM to 2.0 mM as the optimal Mg⁺⁺ range for Taq DNA polymerase, with optimization to be conducted by supplementing the magnesium concentration in 0.5 increments up to 4 mM. See also “Multiplex PCR: Critical Parameters and Step-by-Step Protocol” O. Henegariu, et al., BioTechniques 23:504-511 (September 1997). During development of the technology, it has been discovered that PCR-flap assay buffer reduces bias in multiplex PCR amplification, e.g., in preamplification reactions.

In certain embodiments, the technology relates to using a single label, e.g., a fluorophore to report the aggregate signal from multiplexed amplification of multiple different target nucleic acids amplified with target-specific (e.g., gene-specific) primer pairs in the same probe-based PCR assay (e.g., TAQMAN probe cleavage assay). In some embodiments, an aspect of the technology relates to using a single FRET cassette to report the aggregate signal from multiplexed amplification of multiple different target nucleic acids amplified with target-specific (e.g., gene-specific) primer pairs in the same PCR-flap assay. In preferred embodiments, preamplification conditions are selected that reduce or minimize amplification bias between the different target nucleic acids. In particularly preferred embodiments, conditions are selected that minimize amplification bias without the need to separately optimize the reactions for individual targets, e.g., without the need to adjust concentrations of the different primer pairs to make the amplification efficiencies from different targets in a multiplexed reaction more similar (see, e.g., Wu, et al, Front. Immunol. v11:1631 (2020)). Avoiding a need to separately optimize primer pair concentrations is particularly advantageous in that it simplifies the process of designing primer pairs that can work together in multiplexed assays, and makes highly complex multiplexing feasible, e.g., multiplexing of >20, >30, >40, >50 different amplification targets in a target-enhanced quantitative PCR probe assay such as a PCR-flap assay.

Embodiments of the present technology are directed to combining low-bias multiplexed PCR preamplification with multiplexed PCR-flap assay detection in which multiple markers generate signal using individual dyes, without the need to distinguish between the individual signal contributions from any of the individual targets detected in the multiplexed PCR-flap assays. The process of assaying multiple targets using a single dye in a PCR-flap assay is referred to as Multiple Analyte to one Dye or “MAD” PCR-flap assay. With the option of detecting different dyes in the same reaction (e.g., using additional channels on a fluorescence detector), multiple groups of targets that report to different dyes can be detected in the same PCR-flap assay. The technology provides methods and compositions for assaying large numbers of different target sequences in one or a few multiplexed PCR-flap assays, requiring far fewer PCR flap assay reactions than are used when PCR-flap assay are configured to detect each target sequence individually. The technology also finds use with multiplexed preamplification followed by other multiplexed PCR FRET probe cleavage assays, such a TAQMAN assays.

During development of embodiments of the technology provided herein, it was discovered that use of a preamplification buffer comprising elevated Mg⁺⁺ (e.g., >6 mM, preferably >7 mM, more preferably 7.5 mM) reduces target-to-target amplification bias in highly multiplexed PCR as compared to standard PCR assay conditions. One such buffer is a PCR-flap assay buffer comprising MOPS, Tris-HCl pH 8.0, and 7.5 mM MgCl₂, and little or no added KCl or other monovalent salt. It was further discovered that use of elevated Mg++ in combination with PCR buffers that include reagents that reduce the helix-stabilizing effect of a high Mg⁺⁺ concentration, e.g., DMSO, (NH₄)₂SO₄, betaine, etc., sometimes referred to as enhancer reagents, does not produce the same low-bias effect.

In certain preferred embodiments, the multiple different target nucleic acids are DNA, and the target DNAs are preamplified in a preamplification reaction mixture comprising a mixture of different primer pairs, with each of the primers being in essentially equal concentrations, and at least 6 mM Mg⁺⁺, in a solution comprising MOPS buffer, dNTPs, and a thermostable DNA polymerase. In particularly preferred embodiments, the preamplification reaction mixture comprises 200-600 nM each of different primer pair, with the primers being in essentially equal concentrations, 7.5 mM MgCl₂, 10 mM MOPS pH 7.5, 0.3 mM Tris-HCl, pH 8.0, 0.8 mM KCl, 0.1 μg/μL BSA, 0.0001% TWEEN-20 detergent, 0.0001% IGEPAL CA-630 detergent, 250 μM of each dNTP, and 0.025 units/μL HOTSTART GOTAQ DNA polymerase. In preferred embodiments, preamplification reactions do not contain a labeled probe oligonucleotide, e.g., a TAQMAN probe or a FRET cassette. In particularly preferred embodiments, preamplification reactions do not contain flap assay probes.

In certain preferred embodiments, the multiple different target nucleic acids are RNA, and the target RNAs are preamplified in a preamplification reaction mixture comprising a mixture of different primer pairs, with each of the primers being in essentially equal concentrations, and at least 6 mM Mg⁺⁺, in a solution comprising MOPS buffer, dNTPs, a reverse transcriptase, and a thermostable DNA polymerase. In particularly preferred embodiments, the preamplification reaction mixture comprises 200-600 nM each of different primer pairs, with the primers being in essentially equal concentrations, 0.5-1.0 units/μL of MMLV reverse transcriptase, 7.5 mM MgCl₂, 10 mM MOPS pH 7.5, 0.3 mM Tris-HCl, pH 8.0, 0.8 mM KCl, 0.1 μg/μL BSA, 0.0001% TWEEN-20 detergent, 0.0001% IGEPAL CA-630 detergent, 250 μM of each dNTP, and 0.025 units/μL HOTSTART GOTAQ DNA polymerase. In preferred embodiments, preamplification reactions do not contain a labeled probe oligonucleotide, e.g., a TAQMAN probe or a FRET cassette. In particularly preferred embodiments, preamplification reactions do not contain flap assay probes.

Embodiments of the multiplex preamplification as disclosed herein find use with amplification-based assays, e.g., PCR FRET probe assays (e.g., TAQMAN), PCR-based NGS assays, rolling circle amplification assays, and with PCR-flap assays such as the QuARTS and LQAS assays. As diagrammed in FIG. 1, the QuARTS and LQAS technologies combine a polymerase-based target DNA amplification process with an invasive cleavage-based signal amplification process. Fluorescence signal generated by the QuARTS/LQAS reaction is monitored in a fashion similar to real-time PCR. During each amplification cycle, three sequential chemical reactions occur in each assay well, with the first and second reactions occurring on target DNA templates and the third occurring on a synthetic DNA target labeled with a fluorophore and quencher dyes, thus forming a fluorescence resonance energy transfer (FRET) donor and acceptor pair. The first reaction produces amplified target with a polymerase and oligonucleotide primers, and the second reaction uses a highly structure-specific 5′-flap endonuclease-1 (FEN-1) enzyme reaction to release a 5′-flap sequence from a target-specific oligonucleotide probe that binds to the product of the polymerase reaction, forming an overlap flap substrate. In the third reaction, the cleaved flap anneals to a specially designed oligonucleotide containing a fluorophore and quencher closely linked in a FRET pair such that the fluorescence is quenched (FRET cassette). The released probe flap hybridizes in a manner that forms an overlap flap substrate that allows the FEN-1 enzyme to cleave the 5′-flap containing the fluorophore, thus releasing it from proximity to the quencher molecule. The released fluorophore generates fluorescence signal to be detected. During the second and third reactions, the FEN-1 endonuclease can cut multiple probes per target, generating multiple cleaved 5′-flaps per target, and each cleaved 5′ flap can participate in the cleavage of many FRET cassettes, giving rise to additional fluorescence signal amplification in the overall reaction.

Using a triplex format, each assay is typically designed to detect multiple targets, e.g. 3 genes reporting to 3 distinct fluorescent dyes. See, e.g., Zou, et alt, (2012) “Quantification of Methylated Markers with a Multiplex Methylation-Specific Technology”, Clinical Chemistry 58: 2, incorporated herein by reference for all purposes. In contrast, using the highly multiplexed PCR-flap assays of the present technology, multiple different targets may be assayed together, with the primary flap cleavage products (released probe flaps) generated from each amplified target all reporting to the same FRET cassette. The PCR-flap assays may be further multiplexed by having a second group of targets report to a second FRET cassette, and additional groups of targets report to additional differently labeled FRET cassettes.

### Applications of the Technology

The technology finds application for analyzing multiple nucleic acids in any type of sample or sample mixture, e.g., human or animal cells, tissues, bodily fluids, environmental samples such as soil, water, plant cell or tissue, surface matter, foods or food preparations, etc. Nucleic acids may be analyzed for any types of different number of sequences, e.g., multiple different genes or allelic or epigenetic variants in a sample from a subject, or for amounts of different nucleic acids (e.g., RNA expression products, mutant alleles, or nucleic acids from different microbial or viral strains or variants).

### Methylation and Mutation Marker Analysis

The present technology finds application in assaying differences between multiple nucleic acids, e.g., for detection and measurement of mutations, methylation status, SNPs or other variations, variations between genes or other target nucleic acids. In some embodiments, a marker is a region of 100 or fewer bases, the marker is a region of 500 or fewer bases, the marker is a region of 1000 or fewer bases, the marker is a region of 5000 or fewer bases, or, in some embodiments, the marker is one base. In some embodiments the marker is in a high CpG density promoter.

The technology is not limited by sample type. For example, in some embodiments the sample is a stool sample, a tissue sample, sputum, a blood sample (e.g., plasma, serum, whole blood), an excretion, or a urine sample.

Furthermore, the technology is not limited in the method used to determine methylation state. In some embodiments the assaying comprises using methylation-specific polymerase chain reaction, nucleic acid sequencing, mass spectrometry, methylation-specific nuclease, mass-based separation, or target capture. In some embodiments, the assaying comprises use of a methylation-specific oligonucleotide. In some embodiments, the technology uses massively parallel sequencing (e.g., next-generation sequencing) to determine methylation state, e.g., sequencing-by-synthesis, real-time (e.g., single-molecule) sequencing, bead emulsion sequencing, nanopore sequencing, etc.

In some embodiments, designs for assaying the methylation states of markers comprise analyzing background methylation at individual CpG loci in target regions of the markers to be interrogated by the assay technology. For example, in some embodiments, large numbers of individual copies of marker DNAs (e.g., >10,000, preferably >100,000 individual copies) from samples isolated from subjects diagnosed with disease, e.g., a cancer, are examined to determine frequency of methylation, and these data are compared to a similarly large numbers of individual copies of marker DNAs from samples isolated from subjects without disease. The frequencies of disease-associated methylation and of background methylation at individual CpG loci within the marker DNAs from the samples can be compared, such that CpG loci that have higher signal-to-noise, e.g., higher detectable methylation and/or reduced background methylation, may be selected for use in assay designs. See, e.g., U.S. Pat. Nos. 9,637,792 and 10,519,510, each of which is incorporated herein by reference in its entirety. In some embodiments a group of high signal-to-noise CpG loci (e.g., 2, 3, 4, 5, or more individual CpG loci in a marker region) are co-interrogated by an assay, such that all of the CpG loci must have a pre-determined methylation status (e.g., all must be methylated or none may be methylated) for the marker to be classified as “methylated” or “not methylated” on the basis of an assay result.

Upon evaluating a methylation state, the methylation state is often expressed as the fraction or percentage of individual strands of DNA that is methylated at a particular site (e.g., at a single nucleotide, at a particular region or locus, at a longer sequence of interest, e.g., up to a ˜100-bp, 200-bp, 500-bp, 1000-bp subsequence of a DNA or longer) relative to the total population of DNA in the sample comprising that particular site. Traditionally, the amount of the unmethylated nucleic acid is determined by PCR using calibrators. Then, a known amount of DNA is bisulfite treated and the resulting methylation-specific sequence is determined using either a real-time PCR or other exponential amplification, e.g., a QuARTS assay (e.g., as provided by U.S. Pat. Nos. 8,361,720; 8,715,937; 8,916,344; and 9,212,392, and U.S. patent application Ser. No. 15/841,006).

For example, in some embodiments, methods comprise generating a standard curve for the unmethylated target by using external standards. The standard curve is constructed from at least two points and relates the real-time Ct value for unmethylated DNA to known quantitative standards. Then, a second standard curve for the methylated target is constructed from at least two points and external standards. This second standard curve relates the Ct for methylated DNA to known quantitative standards. Next, the test sample Ct values are determined for the methylated and unmethylated populations and the genomic equivalents of DNA are calculated from the standard curves produced by the first two steps. The percentage of methylation at the site of interest is calculated from the amounts of methylated DNAs relative to the total amount of DNAs in the population, e.g., (number of methylated DNAs)/(the number of methylated DNAs+number of unmethylated DNAs)×100.

Also provided herein are compositions and kits for practicing the methods. For example, in some embodiments, reagents (e.g., primers, probes) specific for one or more markers are provided alone or in sets (e.g., sets of primers pairs for amplifying a plurality of markers). Additional reagents for conducting a detection assay may also be provided (e.g., enzymes, buffers, positive and negative controls for conducting QuARTS, PCR, sequencing, bisulfite, or other assays). In some embodiments, the kits containing one or more reagents necessary, sufficient, or useful for conducting a method are provided. Also provided are reaction mixtures containing the reagents. Further provided are master mix reagent sets containing a plurality of reagents that may be added to each other and/or to a test sample to complete a reaction mixture.

Methods for isolating DNA suitable for these assay technologies are known in the art. In particular, some embodiments comprise isolation of nucleic acids as described in U.S. patent application Ser. No. 13/470,251 (“Isolation of Nucleic Acids”), incorporated herein by reference in its entirely.

Genomic DNA may be isolated by any means, including the use of commercially available kits. Briefly, wherein the DNA of interest is encapsulated by a cellular membrane the biological sample generally is disrupted and lysed by enzymatic, chemical, or mechanical means. The DNA solution may then be cleared of proteins and other contaminants, e.g., by digestion with proteinase K. The genomic DNA is then recovered from the solution. This may be carried out by means of a variety of methods including salting out, organic extraction, or binding of the DNA to a solid phase support. The choice of method will be affected by several factors including time, expense, and required quantity of DNA. All clinical sample types comprising neoplastic matter or pre-neoplastic matter are suitable for use in the present method, e.g., cell lines, histological slides, biopsies, paraffin-embedded tissue, body fluids, stool, colonic effluent, urine, blood plasma, blood serum, whole blood, isolated blood cells, cells isolated from the blood, and combinations thereof.

The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Appl. Ser. No. 61/485,386 or by a related method.

The technology relates to the analysis of any sample that may be associated with cancer, or that may be examined to establish the absence of cancer. For example, in some embodiments the sample comprises a tissue and/or biological fluid obtained from a patient. In some embodiments, the sample comprises a secretion. In some embodiments, the sample comprises sputum, blood, serum, plasma, gastric secretions, lung tissue samples, lung cells or lung DNA recovered from stool. In some embodiments, the subject is human. Such samples can be obtained by any number of means known in the art, such as will be apparent to the skilled person.

While discussed above in regard to analysis of methylation markers, the technology is not limited to methylation analysis, and it is equally applicable to analysis of mutations, allelic variants, or any type of differences between nucleic acids that may be present in a sample comprising or suspected of comprising multiple different nucleic acid targets.

### Detection Assays and Kits

In some embodiments, the markers described herein find use in QUARTS assays performed on stool samples. In some embodiments, methods for producing DNA samples and, in particular, to methods for producing DNA samples that comprise highly purified, low-abundance nucleic acids in a small volume (e.g., less than 100, less than 60 microliters) and that are substantially and/or effectively free of substances that inhibit assays used to test the DNA samples (e.g., PCR, INVADER, QuARTS assays, etc.) are provided. Such DNA samples find use in diagnostic assays that qualitatively detect the presence of, or quantitatively measure the activity, expression, or amount of, a gene, a gene variant (e.g., an allele), or a gene modification (e.g., methylation) present in a sample taken from a patient. For example, some cancers are correlated with the presence of particular mutant alleles or particular methylation states, and thus detecting and/or quantifying such mutant alleles or methylation states has predictive value in the diagnosis and treatment of cancer.

In some embodiments, the sample comprises blood, serum, plasma, or saliva. In some embodiments, the subject is human. Such samples can be obtained by any number of means known in the art, such as will be apparent to the skilled person. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens. The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Nos. 8,808,990 and 9,169,511, and in WO 2012/155072, or by a related method.

### Applications

In some embodiments, nucleic acids present in samples are analyzed to determine relative amounts of nucleic acids from different cells, cell types, microbes, viruses that may be present in a sample, e.g., in an environmental sample such as a soil or water (e.g., fresh water, sea water, wastewater) sample, or other environmental sample. Profiling nucleic acids in such samples finds application in assessing soil and water quality, and for monitoring contamination and the presence or spread of disease. Thus, in some embodiments, the technology finds application in assaying samples for the presence or for relative amounts of nucleic acids by multiplex assaying of a combination of environmental target nucleic acids (e.g., bacterial, viral, etc.), e.g., for detecting the presence, absence, increase, or decrease of particular bacterial or viral species, or genetic variants thereof.

In some embodiments, diagnostic assays identify the presence of a disease or condition in an individual. In some embodiments, the disease is cancer (e.g., lung, pancreatic, HCC, esophageal, stomach, ovarian, etc.).

In some embodiments, the technology finds application in treating a patient (e.g., a patient with cancer, with early-stage cancer, or who may develop cancer), the method comprising determining the methylation state of a multiplex combination of markers as provided herein and administering a treatment to the patient based on the results of determining the methylation state. The treatment may be administration of a pharmaceutical compound, a vaccine, performing a surgery, imaging the patient, performing another test. Preferably, said use is in a method of clinical screening, a method of prognosis assessment, a method of monitoring the results of therapy, a method to identify patients most likely to respond to a particular therapeutic treatment, a method of imaging a patient or subject, and a method for drug screening and development.

In some embodiments, the technology finds application in methods for diagnosing cancer in a subject. The terms “diagnosing” and “diagnosis” as used herein refer to methods by which the skilled artisan can estimate and even determine whether or not a subject is suffering from a given disease or condition or may develop a given disease or condition in the future. The skilled artisan often makes a diagnosis on the basis of one or more diagnostic indicators, such as for example a biomarker, the methylation state of which is indicative of the presence, severity, or absence of the condition.

Along with diagnosis, clinical cancer prognosis relates to determining the aggressiveness of the cancer and the likelihood of tumor recurrence to plan the most effective therapy. If a more accurate prognosis can be made or even a potential risk for developing the cancer can be assessed, appropriate therapy, and in some instances less severe therapy for the patient can be chosen. Assessment (e.g., determining methylation state, the presence of mutations) of cancer biomarkers is useful to separate subjects with good prognosis and/or low risk of developing cancer who will need no therapy or limited therapy from those more likely to develop cancer or suffer a recurrence of cancer who might benefit from more intensive treatments.

As such, “making a diagnosis” or “diagnosing”, as used herein, is further inclusive of determining a risk of developing cancer or determining a prognosis, which can provide for predicting a clinical outcome (with or without medical treatment), selecting an appropriate treatment (or whether treatment would be effective), or monitoring a current treatment and potentially changing the treatment, based on the measure of the diagnostic biomarkers disclosed herein.

Further, in some embodiments of the technology, multiple determinations of the biomarkers over time can be made to facilitate diagnosis and/or prognosis. A temporal change in the biomarker can be used to predict a clinical outcome, monitor the progression of cancer, and/or monitor the efficacy of appropriate therapies directed against the cancer. In such an embodiment for example, one might expect to see a change in the methylation state of one or more biomarkers disclosed herein (and potentially one or more additional biomarker(s), if monitored) in a biological sample over time during the course of an effective therapy.

The technology further finds application in methods for determining whether to initiate or continue prophylaxis or treatment of a cancer in a subject. In some embodiments, the method comprises providing a series of biological samples over a time period from the subject; analyzing the series of biological samples to determine a methylation state of at least one biomarker disclosed herein in each of the biological samples; and comparing any measurable change in the methylation states of one or more of the biomarkers in each of the biological samples. Any changes in the methylation states of biomarkers over the time period can be used to predict risk of developing cancer, predict clinical outcome, determine whether to initiate or continue the prophylaxis or therapy of the cancer, and whether a current therapy is effectively treating the cancer. For example, a first time point can be selected prior to initiation of a treatment and a second time point can be selected at some time after initiation of the treatment. Methylation states can be measured in each of the samples taken from different time points and qualitative and/or quantitative differences noted. A change in the methylation states of the biomarker levels from the different samples can be correlated with risk for developing cancer, prognosis, determining treatment efficacy, and/or progression of the cancer in the subject.

In preferred embodiments, the methods and compositions of the invention are for treatment or diagnosis of disease at an early stage, for example, before symptoms of the disease appear. In some embodiments, the methods and compositions of the invention are for treatment or diagnosis of disease at a clinical stage.

As noted above, in some embodiments, multiple determinations of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular type or severity of cancer, or a given prognosis. Likewise, a decrease in the marker from the initial time to the second time can be indicative of a particular type or severity of cancer, or a given prognosis. Furthermore, the degree of change of one or more markers can be related to the severity of the cancer and future adverse events. The skilled artisan will understand that, while in certain embodiments comparative measurements can be made of the same biomarker at multiple time points, one can also measure a given biomarker at one time point, and a second biomarker at a second time point, and a comparison of these markers can provide diagnostic information.

As used herein, the phrase “determining the prognosis” refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the methylation state of a biomarker. Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition, the chance of a given outcome (e.g., suffering from cancer) may be very low.

In some embodiments, a statistical analysis associates a prognostic indicator with a predisposition to an adverse outcome. For example, in some embodiments, a methylation state different from that in a normal control sample obtained from a patient who does not have a cancer can signal that a subject is more likely to suffer from a cancer than subjects with a level that is more similar to the methylation state in the control sample, as determined by a level of statistical significance. Additionally, a change in methylation state from a baseline (e.g., “normal”) level can be reflective of subject prognosis, and the degree of change in methylation state can be related to the severity of adverse events. Statistical significance is often determined by comparing two or more populations and determining a confidence interval and/or ap value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In other embodiments, a threshold degree of change in the methylation state of a prognostic or diagnostic biomarker disclosed herein can be established, and the degree of change in the methylation state of the biomarker in a biological sample is simply compared to the threshold degree of change in the methylation state. A preferred threshold change in the methylation state for biomarkers provided herein is about 5%, about 10%, about 15%, about 20%, about 25%, about 30%, about 50%, about 75%, about 100%, and about 150%. In yet other embodiments, a “nomogram” can be established, by which a methylation state of a prognostic or diagnostic indicator (biomarker or combination of biomarkers) is directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments, a control sample is analyzed concurrently with the biological sample, such that the results obtained from the biological sample can be compared to the results obtained from the control sample. Additionally, it is contemplated that standard curves can be provided, with which assay results for the biological sample may be compared. Such standard curves present methylation states of a biomarker as a function of assay units, e.g., fluorescent signal intensity, if a fluorescent label is used. Using samples taken from multiple donors, standard curves can be provided for control methylation states of the one or more biomarkers in normal tissue, as well as for “at-risk” levels of the one or more biomarkers in tissue taken from donors with cancer.

Assays of the technology can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

In some embodiments, the subject is diagnosed as having cancer if, when compared to a control methylation state, there is a measurable difference in the methylation state of at least one biomarker in the sample. Conversely, when no change in methylation state is identified in the biological sample, the subject can be identified as not having cancer, not being at risk for the cancer, or as having a low risk of the cancer. In this regard, subjects having cancer or risk thereof can be differentiated from subjects having low to substantially no cancer or risk thereof. Those subjects having a risk of developing cancer can be placed on a more intensive and/or regular screening schedule. On the other hand, those subjects having low to substantially no risk may avoid being subjected to screening procedures, until such time as a future screening, for example, a screening conducted in accordance with the present technology, indicates that a risk of cancer has appeared in those subjects.

As mentioned above, depending on the embodiment of the method of the present technology, detecting a change in methylation state of the one or more biomarkers can be a qualitative determination or it can be a quantitative determination. As such, the step of diagnosing a subject as having, or at risk of developing, cancer indicates that certain threshold measurements are made, e.g., the methylation state of the one or more biomarkers in the biological sample varies from a predetermined control methylation state. In some embodiments of the method, the control methylation state is any detectable methylation state of the biomarker. In other embodiments of the method where a control sample is tested concurrently with the biological sample, the predetermined methylation state is the methylation state in the control sample. In other embodiments of the method, the predetermined methylation state is based upon and/or identified by a standard curve. In other embodiments of the method, the predetermined methylation state is a specific state or range of state. As such, the predetermined methylation state can be chosen, within acceptable limits that will be apparent to those skilled in the art, based in part on the embodiment of the method being practiced and the desired specificity, etc.

### Liquid Biopsy

Over recent years, it has become apparent that circulating epithelial cells, representing metastatic tumor cells, can be detected in the blood of many patients with cancer. Molecular profiling of rare cells is important in biological and clinical studies. Applications range from characterization of circulating epithelial cells (CEpCs) in the peripheral blood of cancer patients for disease prognosis and personalized treatment (See e.g., Cristofanilli M, et al. (2004) N Engl J Med 351:781-791; Hayes D F, et al. (2006) Clin Cancer Res 12:4218-4224; Budd G T, et al., (2006) Clin Cancer Res 12:6403-6409; Moreno J G, et al. (2005) Urology 65:713-718; Pantel et al., (2008) Nat Rev 8:329-340; and Cohen S J, et al. (2008) J Clin Oncol 26:3213-3221). Accordingly, embodiments of the present disclosure provide compositions and methods for detecting the presence of metastatic cancer in a subject by identifying the presence of methylation markers in plasma or whole blood.

### EXPERIMENTAL EXAMPLES

The following examples are offered to illustrate but not to limit the invention. In order to facilitate understanding, the specific embodiments are provided to help interpret the technical proposal, that is, these embodiments are only for illustrative purposes, but not in any way to limit the scope of the invention. Unless otherwise specified, embodiments that do not indicate the specific conditions, are in accordance with the conventional conditions or the manufacturer's recommended conditions.

### Example 1

### Sample Preparation, Preamplification, And PCR-Flap Assays

### Sample Preparation Methods

Exemplary methods of isolating RNA and DNA from samples, e.g., cell, plasma, or blood cells, and optionally treating DNA, are described in detail in WO 2021/041726, filed 27 Aug. 2020, which is incorporated herein by reference for all purposes. Optionally, DNA from samples may be treated with a methylation-specific reagent, e.g., a bisulfite reagent or using the TAPS method combining oxidation by TET enzymes with reduction by borane derivatives, as described herein above. The converted DNA is then used in a detection assay, e.g., a preamplification and/or flap endonuclease assays, as described below. For additional embodiments of bisulfite treatment of nucleic acids, also U.S. Pat. No. 10,704,081, and U.S. Patent Appl. Ser. Nos. 63/058,179, filed Jul. 29, 2020, each of which is incorporated herein by reference in its entirety, for all purposes, and which may be applied in the technology described herein.

In some embodiments, RNA and DNA are isolated from different samples of blood from a subject. For example, blood may be collected in a first collection tube configured for optimal preservation and/or isolation of RNA and in a second collection tube configured to optimal preservation and isolation of DNA, and the RNA and DNA may be extracted from portions of blood collected in this fashion. In other embodiments, RNA and DNA are both extracted from a single collected blood sample, using, e.g., a collection tube configured to optimal preservation and isolation of both DNA and RNA (e.g., cf-DNA/cf-RNA Preservative Tubes (Cat. 63950) from NORGEN Biotek Corp., for preservation and isolation of both cell-free DNA and cell-free RNA).

In some embodiments, RNA and DNA are assayed together, e.g., in an RT-LQAS/RT-TELQAS reaction. In some embodiments, the RNA and DNA are separately isolated and/or separately treated, e.g., with bisulfite, as described above, while in some embodiments, RNA and DNA are processed together, e.g., both being present during bisulfite treatment and subsequent purification, and added together to the assay reactions.

### Flap Endonuclease Assays

The QuARTS and LQAS flap assay technologies combine a polymerase-based target DNA amplification process with an invasive cleavage-based signal amplification process. The QuARTS technology is described, e.g., in U.S. Pat. Nos. 8,361,720; 8,715,937; 8,916,344; and 9,212,392, and a flap assay using probe oligonucleotides having a longer target-specific region (Long probe Quantitative Amplified Signal, “LQAS”) is described in U.S. Pat. No. 10,648,025, each of which is incorporated herein by reference in its entirety for all purposes. A combined preamplification and LQAS assay is referred to as the “TELQAS” assay (for “Target Enrichment Long probe Quantitative Amplified Signal”).

In QuARTS assays, the flap probe oligonucleotides have a target-specific region of 12 bases, while the LQAS assays use flap oligonucleotides having a target specific region of at least 13 bases, and use different thermal cycling procedures for amplification. Fluorescence signal generated by the QuARTS and LQAS reactions are monitored in a fashion similar to real-time PCR, permitting quantitation of the amount of a target nucleic acid in a sample.

An exemplary QuARTS reaction typically comprises approximately 200-600 nmol/L (e.g., 500 nmol/L) of each primer and detection probe, approximately 100 nmol/L of the invasive oligonucleotide, approximately 600-700 nmol/L of each FRET cassette (FAM, e.g., as supplied commercially by Hologic, Inc.; HEX, e.g., as supplied commercially by BioSearch Technologies; and Quasar 670 (“Q670”), e.g., as supplied commercially by BioSearch Technologies, and comprising a “black hole” quencher, e.g., BHQ-1, BHQ-2, or BHQ-3, BioSearch Technologies), 6.675 ng/μL FEN-1 endonuclease (e.g., Cleavase® 2.0, Hologic, Inc.), 1 unit Taq DNA polymerase in a 30 μL reaction volume (e.g., GoTaq® DNA polymerase, Promega Corp., Madison, WI), 10 mmol/μL 3-(n-morpholino) propanesulfonic acid (MOPS), 7.5 mmol/L MgCl₂, and 250 μmol/L of each dNTP. Exemplary QuARTS cycling conditions are as shown in the table below. In some applications, analysis of the quantification cycle (C_{q}) provides a measure of the initial number of target DNA strands (e.g., copy number) in the sample.

| Stage | Temp/Time | # of Cycles |
|---|---|---|
| Denaturation/Polymerase activation | 95° C./3′ | 1 |
| Amplification Phase 1 | 95° C./20″ | 10 |
| | 67° C./30″ | |
| | 70° C./30″ | |
| Amplification Phase 2 | 95° C./20″ | 37 |
| | 53° C./1′ | |
| | 70° C./30″ | |
| Cooling | 40° C./30″ | 1 |

It should be noted that the Amplification phases 1 and 2 are not separate amplification reactions, but describe the incubation of a single reaction mixture that is cycled through one thermal profile for the first 10 cycles, then a different thermal profile for the following thermal cycles (37 cycles, in this example). The two amplification phases are conducted in sequence on the same reaction mixture without, for example, making additions to or other alterations to the contents of reaction mixture.

An exemplary LQAS reaction typically comprises approximately 200-600 nmol/L of each primer, approximately 100 nmol/L of the invasive oligonucleotide, approximately 500 nmol/L of each flap oligonucleotide probe and FRET cassette. LQAS reactions may, for example, be subjected to the following thermocycling conditions:

| Stage | Temp/Time | # of Cycles |
|---|---|---|
| Denaturation/Polymerase activation | 95° C./3′ | 1 |
| Amplification | 95° C./20″ | 40 |
| | 63° C./1′ | |
| | 70° C./30″ | |
| Cooling | 40° C./30″ | 1 |

WO 2021/041726 further describes exemplary QuARTS and LQAS/TELQAS flap assay methods that combine a polymerase-based target DNA amplification process with an invasive cleavage-based signal amplification process. Fluorescence signal generated by the QuARTS and LQAS reactions are monitored in a fashion similar to real-time PCR, permitting quantitation of the amount of a target nucleic acid in a sample.

Also contemplated are assays comprising multiplex reverse transcription and preamplification, followed by LQAS PCR-flap assays (a combined reverse transcription and preamplification with an LQAS assay is referred to as the RT-TELQAS assay for “Reverse Transcription-Target Enrichment Long probe Quantitative Amplified Signal”). In RT-TELQAS assays, target RNAs, e.g., total RNA from a sample, is treated in an RT-preamplification reaction containing, e.g., 20 U of MMLV reverse transcriptase, 1.5 U of GoTaq® DNA Polymerase,10 mM MOPS buffer, pH7.5, 7.5 mM MgCl₂, 250 μM each dNTP, and oligonucleotide primers (e.g., for 12 targets, 12 primer pairs/24 primers, in equimolar amounts (e.g., 200 nM each primer) or in amounts modified to adjust amplification efficiencies of different target RNAs, and is incubated at a moderate temperature (e.g., 42° C.) for reverse transcription, followed by a limited number of thermal cycles (e.g., 10 cycles of 95° C., 63° C., 70° C.) to provide preamplification of target sequences corresponding to the included primers pairs. After thermal cycling, aliquots of the RT-preamplification reaction (e.g., 10 μL) are used in LQAS PCR-flap assays, as described below. RNAs suitable for detection in RT-TELQAS and RT-LQAS assays are not limited to any particular types of RNA targets. For example all manner of RNAs from tissues, cells or circulating cell-free RNAs from blood, such as protein-coding messenger RNAs (mRNA), microRNAs (miRNAs), piRNAs, tRNAs, and other non-coding RNA molecules (ncRNAs) (see, e.g., SU Umu, et al. “A comprehensive profile of circulating RNAs in human serum,” RNA Biology 15(2):242-250 (2018), which is incorporated herein by reference in its entirety) may be assayed using the RT-TELQAS and RT-LQAS methods described hereinbelow.

### Multiplexed Preamplification of Sample DNA

To preamplify most or all of the target DNA from an input sample (e.g., DNA isolated from a sample, or cDNA produced from an isolated RNA), a large volume of the isolated DNA may be used in a single multiplex amplification reaction. A preamplification is conducted, for example, in a reaction mixture containing 7.5 mM MgCl₂, 10 mM MOPS, 0.3 mM Tris-HCl, pH 8.0, 0.8 mM KCl, 0.1 μg/μL BSA, 0.0001% Tween-20, 0.0001% IGEPAL CA-630, 250 μM each dNTP, oligonucleotide primers, (e.g., for 20 targets, 20 primer pairs/40 primers, in essentially equimolar amounts, including but not limited to the ranges of, e.g., 200-600 nM each primer), 0.025 units/μL HotStart GoTaq concentration, and 20 to 50% by volume of sample DNA (e.g., 10 μL of target DNA into a 50 μL reaction mixture, or 50 μL of target DNA into a 125 μL reaction mixture).

Thermal cycling times and temperatures are selected to be appropriate for the volume of the reaction and the amplification vessel. For example, the reactions may be cycled as follows:

| Stage | Temp/Time | #of Cycles |
|---|---|---|
| Pre-incubation | 95° C./5 min. | 1 |
| Amplification | 95° C./30 sec. | 10-12 |
| | 64° C./30 sec. | |
| | 72° C./30 sec. | |
| Cooling | 4° C./Hold | 1 |

After thermal cycling, aliquots of the preamplification reaction (e.g., 10 μL) are typically diluted (e.g., into 500 μL in 10 mM Tris, 0.1 mM EDTA), with or without bulk endogenous DNA (e.g., fish DNA for minimizing variation in polymerase activity). Aliquots of the diluted preamplified DNA (e.g., 10 μL) or of the undiluted preamplification reaction are used in a multiplex PCR-flap assay using all of, or a subset of, the same primer pairs.

### Multiplexed Preamplification of Sample RNAs

A preamplification from isolated RNA or mixed RNA+DNA sample is conducted for example, in a reaction mixture containing 7.5 mM MgCl₂, 10 mM MOPS, 0.3 mM Tris-HCl, pH 8.0, 0.8 mM KCl, 0.1 μg/μL BSA, 0.0001% Tween-20, 0.0001% IGEPAL CA-630, 250 μM each dNTP, oligonucleotide primers, (e.g., for 20 targets, 20 primer pairs/40 primers, in essentially equimolar amounts, including but not limited to the ranges of, e.g., 200-600 nM each primer), 0.025 units/μL HOTSTART GOTAQ DNA polymerase, 0.67 units/μL of MMLV reverse transcriptase, and 20 to 50% by volume of sample RNA (e.g., 10 μL of target RNA into a 50 μL reaction mixture, or 50 μL of target RNA into a 125 μL reaction mixture).

Thermal cycling times and temperatures are selected to be appropriate for the volume of the reaction and the amplification vessel. For example, the reactions may be cycled as follows:

RT-Preamplification Reaction Thermal Profile

| Stage | Temp/Time | #of Cycles |
|---|---|---|
| Reverse transcription | 42° C./30′ | 1 |
| | 95° C./3′ | 1 |
| Amplification | 95° C./20″ | 10 |
| | 63° C./30″ | |
| | 70° C./30″ | |
| Cooling | 4° C./Hold | 1 |

After thermal cycling, aliquots of the preamplification reaction (e.g., 10 μL) are typically diluted (e.g., into 500 μL in 10 mM Tris, 0.1 mM EDTA), with or without bulk endogenous DNA (e.g., fish DNA for minimizing variation in polymerase activity). Aliquots of the diluted preamplified DNA (e.g., 10 μL) or of the undiluted preamplification reaction are used in a multiplex PCR-flap assay using all of, or a subset of, the same primer pairs. Diluted and undiluted preamplified samples may be stored at −20° C.

PCR-Flap Assays from Multiplexed Preamplified DNA

Using a preamplified sample as discussed above, QuARTS and LQAS PCR-flap assay reactions are typically set up as follows:

| | Volume per |
|---|---|
| Master Mix (per reaction) | reaction (μL) |
| Water (mol. biol. grade) | 15.50 |
| 10X Oligo Mix* | 3.00 |
| 20X QuARTS/LQAS Enzyme Mix** | 1.50 |
| Total Master mix volume | 20.0 |

Reaction Mix

| | |
|---|---|
| Master mix | 20 |
| Diluted preamplified sample | 10 |
| Final Reaction volume | 30 |

*10X oligonucleotide mix = 2 μM each primer and 5 μM each probe and FRET oligonucleotide
**20X enzyme mix contains 1 unit/μL GoTaq Hot start polymerase (Promega Corp.), 292 ng/μL Cleavase 2.0 FEN-1 flap endonuclease (Hologic, Inc.), 200 mM MOPS, pH 7.5, 150 mM MgCl2, 6.38 mM Tris-HCl, pH 8.0, 15.95 mM KCl, 2 μg/μL BSA, 0.16% Tween-20, 0.16% IGEPAL CA-630, 25% Glycerol.

As noted above, the flap oligonucleotides in the QuARTS assays have a target-specific region of 12 bases, while the LQAS assays use flap oligonucleotides have a target-specific region of at least 13 bases and are subjected to different thermal cycling conditions. In some embodiments, QuARTS reactions are subjected to the following thermocycling conditions:

| QuARTS Assay Reaction Cycle: | | | | |
|---|---|---|---|---|
| | | Ramp | | |
| | | Rate | Number | |
| | | (° C. per | of | Signal |
| Stage | Temp/Time | second) | Cycles | Acquisition |
| Pre-incubation | 95° C./3 min | 4.4 | 1 | No |
| Amplification | 95° C./20 sec | 4.4 | 5 | No |
| Phase 1 | 63° C./30 sec | 2.2 | | No |
| | 70° C./30 sec | 4.4 | | No |
| Amplification | 95° C./20 sec | 4.4 | 40 | No |
| Phase 2 | 53° C./1 min | 2.2 | | Yes |
| | 70° C./30 sec | 4.4 | | No |
| Cooling | 40° C./30 sec | 2.2 | 1 | No |

In some embodiments, LQAS PCR-flap assay reactions are subjected to the following thermocycling conditions:

| LQAS Assay Reaction Cycle: | | | | |
|---|---|---|---|---|
| | | Ramp | | |
| | | Rate | Number | |
| | | (° C. per | of | Signal |
| Stage | Temp/Time | second) | Cycles | Acquisition |
| Pre-incubation | 95° C./3 min | 4.4 | 1 | No |
| Amplification | 95° C./20 sec | 4.4 | 40 | No |
| | 63° C./1 min | 2.2 | | Yes |
| | 70° C./30 sec | 4.4 | | No |
| Cooling | 40° C./30 sec | 2.2 | 1 | No |

### Example 2

### Effect of Magnesium Concentration of PCR-Flap Assay Buffer on Amplification Bias in Multiplexed Preamplification

The effect of using buffers having different concentrations of Mg⁺⁺ (provided, e.g., as MgCl₂) on amplification bias in multiplexed reactions was investigated. A PCR-flap assay buffer modified for multiplexing was used as the basis for testing different concentrations of Mg⁺⁺. 10× concentrated multiplex PCR-flap assay buffers were prepared with different concentrations of MgCl₂, to produce final concentrations of either 7.5 mM MgCl₂ (the standard high-Mg⁺⁺ concentration of PCR-flap assay buffer) or to produce a final concentration of 2.5 mM MgCl₂ (the low-Mg⁺⁺ concentration in the range typically used in PCR assays). The 10× buffers were otherwise identical and contained 100 mM MOPS, pH 7.5; 0.08% Tween 20; 0.08% IGEPAL-CA630 and 2.5 mM each dNTP, in addition to either 75 or 25 mM MgCl₂.

A multiplex calibrator containing equal amounts of 37 different markers (a plasmid comprising the different target sequences, digested to provide equimolar amounts of 37 individual marker DNAs) was diluted in 10 mM Tris HCl, 0.1 mM EDTA to produce preparations containing 2 or 20 strands of target DNA per μL, to provide either 100 strands of target DNA or 1000 strands of target DNA per 50 μL of diluted stock. The target DNAs were preamplified at using the high or low concentrations of MgCl₂.

Each preamplification reaction contained 25 μL of the following Master Mix, which was combined with 50 μL of the diluted sample DNA for a final preamplification reaction mixture volume of 75.0 μL. The sample DNA aliquots contained either 1000 strands per reaction of the calibrator plasmid or 100 strands per reaction of the calibrator plasmid, as described above.

| | Vol. per Reaction |
|---|---|
| Master Mix Component | (μL) |
| Molecular Biol. Grade H₂O | 8.50 |
| 10X multiplex PCR-flap assay buffer | 7.50 |
| 0.75 μM Primer Mix | 7.50 |
| 1.67M KCl with 1.67 mg/mL BSA | 1.12 |
| HotStart GoTaq (5 units/μL) | 0.375 |
| Total Vol. Master Mix (μL) | 25.0 |
| Sample DNA (calibrator plasmids at 2 or 20 | 50 |
| strands/μL) | |
| Or No Target Control (“NTC”; 20 ng/μL PolyA | |
| (Roche PN #: 10108626001) in Tris-EDTA. ) | |
| Final PreAMP Rxn Vol | 75.0 |

*The Primer Mix formulation contains 0.75 μM of each of the forward (FP) and reverse primer (RP) for each of the marker DNAs listed below:

| | Marker DNA |
|---|---|
| 1 | ARHGEF4 |
| 2 | B3GALT6 |
| 3 | CAPN2 |
| 4 | CDO1 |
| 5 | CHST2 |
| 6 | EMX1 |
| 7 | FAIM2 |
| 8 | FAM59B |
| 9 | GPRIN1 |
| 10 | HOXA9 |
| 11 | NDRG4 |
| 12 | PPP2R5C |
| 13 | PRKCB |
| 14 | QKI |
| 15 | SFMBT2 |
| 16 | SIM2 |
| 17 | TSPYL5 |
| 18 | ZNF671 |
| 19 | ZNF781 |

Each reaction was performed in duplicate.

The preamplification reactions were assembled in a 96 well and amplified using the following thermal profile:

Preamplification Reaction Thermal Profile:

| Stage | Temp/Time | # Cycles |
|---|---|---|
| Denaturation/Polymerase | 95° C./5′ | 1 |
| activation | | |
| Amplification | 95° C./30″ | 12 |
| | 64° C./1′ | |
| Cooling | 4° C./Hold | 1 |

20 μL aliquots of preamplification reactions were diluted into 180 μL of 10 mM Tris-Cl, 0.1 mM EDTA, and the undiluted and diluted preamplification products were stored at−20° C.

### LQAS PCR-Flap Assay Reactions

After preamplification in the two different buffers described above, the preamplified product as assayed using triplex PCR-flap assays. The B3GALT6 marker was used as an internal reference target for quantifying the 18 other marker DNAs in triplex reactions described below.

Ten μL of diluted preamplified sample were analyzed in each of 14 different Triplex PCR flap assays, each assembled as follows:

| | Vol. per Reaction |
|---|---|
| Master Mix Component | (μL) |
| Molecular Biol. Grade H₂O | 15.5 |
| 10X Triplex Oligo Mix | 3 |
| 20X Enzyme Mix 2X Cleavase | 1.5 |
| Total Vol. Master Mix (μL) | 20.0 |
| Sample DNA (Preamplified product) | 10 |
| Final LQAS Rxn Vol | 30.0 |

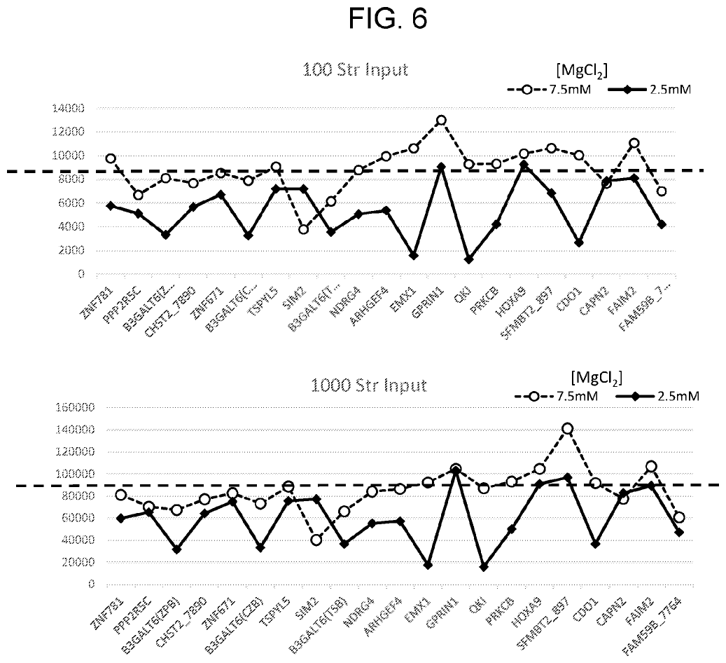

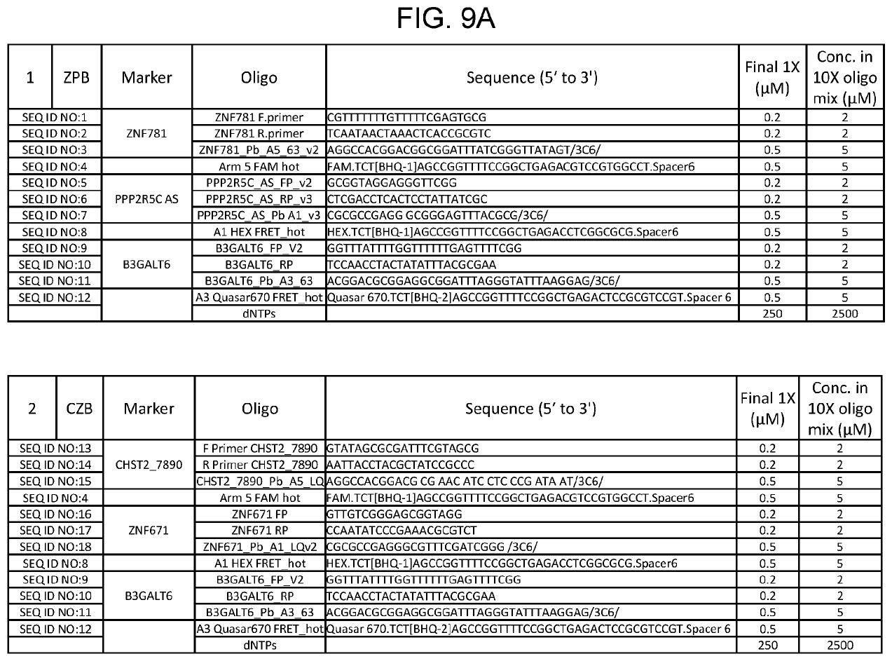

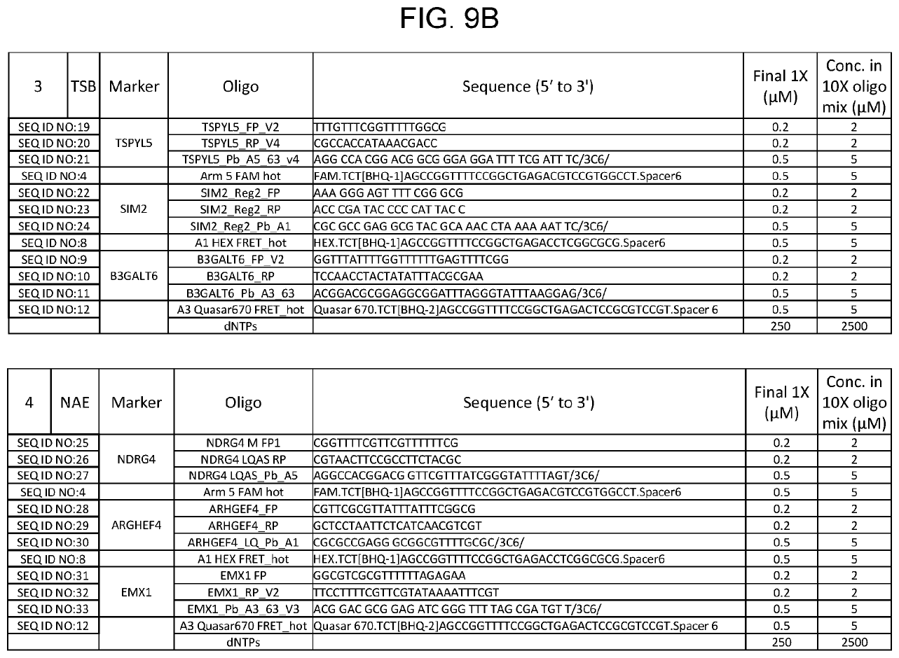

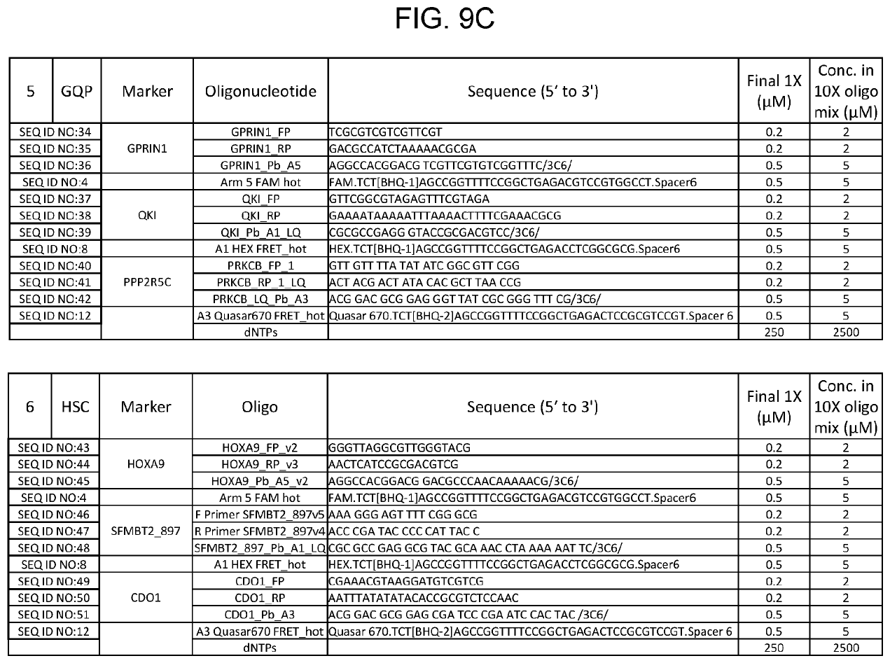

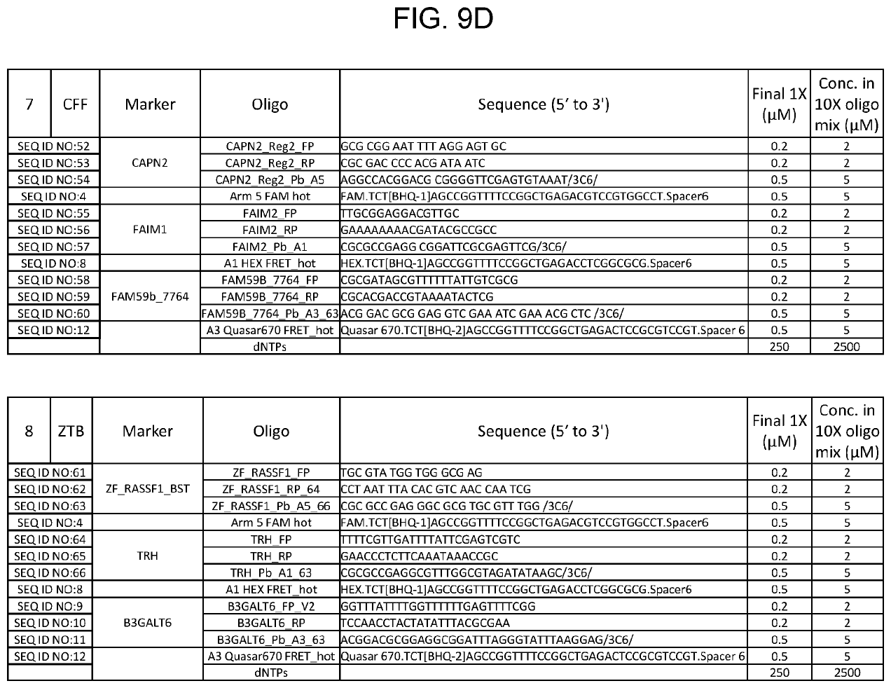

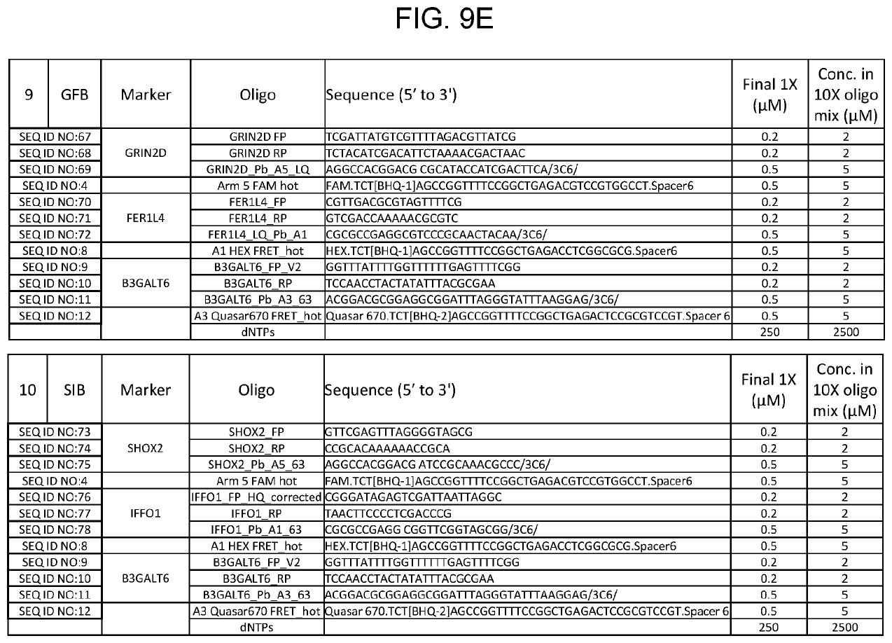

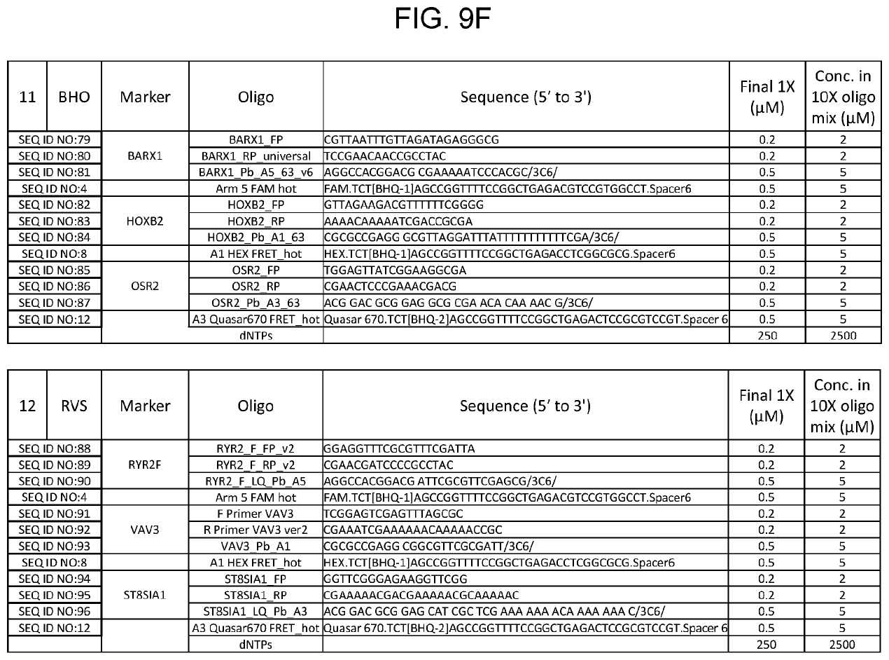

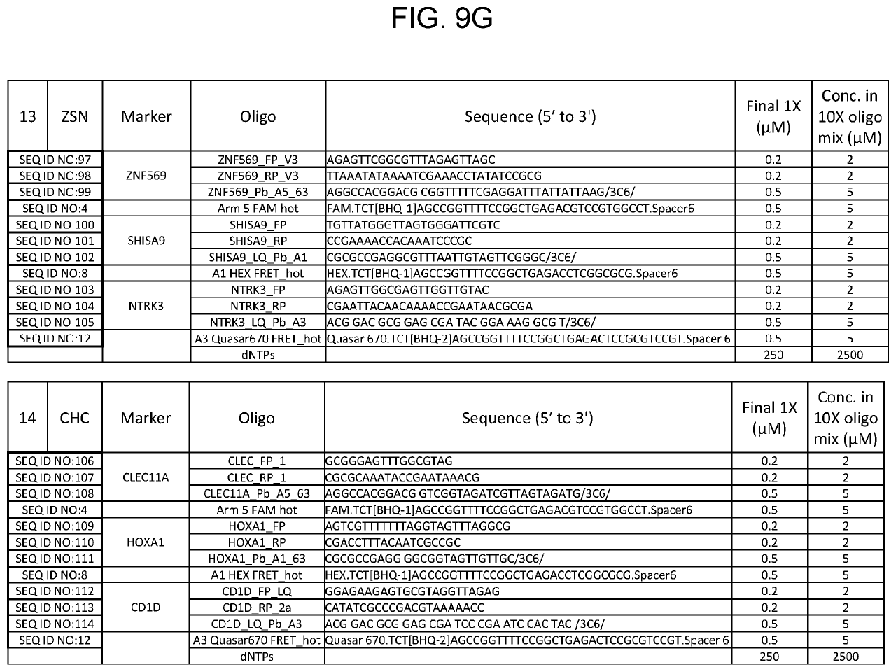

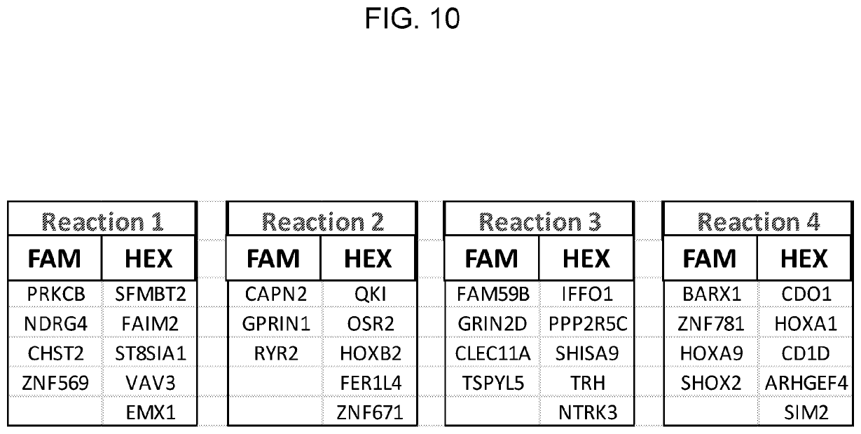

*10X oligonucleotide mix = 2 μM each primer and 5 μM each probe and FRET oligonucleotide. See FIG. 6 for the different combinations of markers in each triplex and FIG. 9A-9G for formulations of each 10X triplex oligonucleotide mixes listed in the table below.
**20X enzyme mix contains 1 unit/μL GoTaq Hot start polymerase (Promega Corp.), 292 ng/μL Cleavase 2.0 FEN-1 flap endonuclease (Hologic, Inc.), 200 mM MOPS, pH 7.5, 150 mM MgCl2, 6.38 mM Tris-HCl, pH 8.0, 15.94 mM KCl, 2 μg/μL BSA, 0.16% Tween-20, 0.16% IGEPAL CA-630, 25% Glycerol.

Reactions were assembled to include primer pairs for the markers in the following triplex combinations:

Triplex reactions

| Assay | A5-FAM | A1-HEX | A3-Q670 |
|---|---|---|---|
| Name | FRET Cassette | FRET Cassette | FRET Cassette |
| ZPB | ZNF781 | PPP2R5C | B3GALT6 |
| CZB | CHST2 | ZNF671 | B3GALT6 |
| TSB | TSPYL5 | SIM2 | B3GALT6 |
| NAE | NDRG4 | ARHGEF4 | EMX1 |
| GQP | GPRIN1 | QKI | PRKCB |
| HSC | HOXA9 | SFMBT2 | CDO1 |
| CFF | CAPN2 | FAIM2 | FAM59B |

Each reaction was performed in duplicate. Results are shown in FIG. 6, with calculated theoretical yield being indicated by the horizontal straight dashed line. The amount of deviation above or below the dashed line indicates the amount of variation in signal seen for each marker at each concentration of Mg⁺⁺.

These data show that the average coefficient of variance of the strands was reduced from 43% when 2.5 mM MgCl₂ was used to 22% when 7.5 mM MgCl₂ was used in the preamplification reactions. Amplification efficiency was increased from an average of 70% at 2.5 mM MgCl₂ to an average of 106% when 7.5 mM MgCl₂ was used in the preamplification reactions. These data show that use of high Mg⁺⁺ PCR-flap assay buffer in the multiplex PCR preamplification both reduced amplification bias between the different targets and increased the signal from each target in follow-on LQAS PCR-flap assay reactions.

### Example 3

### Comparing Amplification Bias in Multiplexed Preamplification in Low-Bias PCR-Flap Assay Buffer or (NH₄)₂SO₄ PCR Buffer

Some methods use (NH₄)₂SO₄ in PCR buffer to reduce the duplex-stabilizing effect of excess Mg⁺⁺ ions to, for example, broaden the range of magnesium that can be used without increasing the background in the PCR that is typically seen when too much Mg⁺⁺ is present in the amplification reaction. See, e.g., MM Blanchard, et al., PCR buffer optimization with uniform temperature regimen to facilitate automation. Genome Res. 2: 234-240 (1993). PCR buffers that comprise (NH₄)₂SO₄ alone or with other helix-destabilizing additives may sometimes comprise Mg⁺⁺ at concentrations above the typical 1 to 4 mM range commonly used for PCR. One such buffer consists of 16.6 mM (NH₄)₂SO₄, 67 mM Tris-Cl pH 8.8, 6.7 mM MgCl₂, 10 mM β-mercaptoethanol, and 0.1% DMSO. See, e.g., Sukumar, et al., US 2005/0239101A1; Fackler et al., Cancer Research 64: 4442-44452 (2004); Herman et al., Proc. Natl. Acad. Sci. USA Vol. 93, pp. 9821-9826, September 1996). This buffer is disclosed for use with 1.25 mM dNTPs, 2.5 to 5 units Platinum Taq polymerase, 100 ng of each forward and reverse primer in a 25 μL reaction with bisulfite treated DNA. (see, e.g., Sukumar, supra)

Multiplex preamplification in the low-bias PCR-flap assay buffer discussed above was compared to multiplex preamplification in the (NH₄)₂SO₄ PCR buffer described above, with all preamplification reactions assayed using triplex PCR-flap assays in PCR-flap assay buffer, as described above.

Two multiplex master mixes were assembled as follows:

| | |
|---|---|
| | Final Reaction |
| Master Mix Component | Concentration |

(NH₄)₂SO₄ PCR buffer preamplifications:

| | |
|---|---|
| Primer Mix (μM) | 0.075 |
| Total dNTPs (μM) | 1250.0 |
| MgCl₂ (mM) | 6.70 |
| (NH₄)₂SO₄ | 0.017 |
| Tris-HCL (mM) (pH 8.8) | 67.000 |
| B-mercaptoethanol* (M) | 0.010 |
| DMSO (% v:v) | 0.100 |
| HotStart GoTaq (units/μL) | 0.033 |

Low-bias PCR-flap assay buffer preamplifications:

| | |
|---|---|
| Primer Mix (μM) | 0.075 |
| Total dNTPs (μM) | 1.0 |
| MgCl₂ (mM) | 7.5 |
| MOPS (mM) | 10 |
| KCl (mM) | 25 |
| BSA (% w:v) | 0.025 |
| Tween 20 (% v:v) | 0.008% |
| IGEPAL-CA-630 (% v:v) | 0.008% |
| HotStart GoTaq(units/μL) | 0.025 |

For each preamplification reaction, a 3× master mix was assembled in a volume of 25 μL, which was combined with 50 μL of target DNA for a final reaction volume of 75 μL having the final reaction concentrations shown above.

The reactions were amplified using the following thermal profile:

Preamplification Reaction Thermal Profile:

| Stage | Temp/Time | # Cycles |
|---|---|---|
| Denaturation/Polymerase | 95° C./5′ | 1 |
| activation | | |
| Amplification | 95° C./30″ | 12 |
| | 64° C./1′ | |
| Cooling | 4° C./Hold | 1 |

20 μL aliquots of preamplification reactions were diluted into 180 μL 10 mM Tris-Cl, 0.1 mM EDTA and the undiluted and diluted preamplification products were stored at −20° C.

### LQAS PCR-Flap Assay Reactions

Eighteen different methylation markers and B3GALT6 internal reference marker were quantified using the triplex LQAS format described above. Ten μL aliquots of diluted preamplified sample were analyzed in each of 14 different Triplex PCR flap assays, each assembled as follows

| | Vol. per Reaction |
|---|---|
| Master Mix Component | (μL) |
| Molecular Biol. Grade H₂O | 15.5 |
| 10X Triplex Oligo Mix | 3 |
| 20X Enzyme Mix 2X Cleavase | 1.5 |
| Total Vol. Master Mix (μL) | 20.0 |
| Sample DNA (Preamplified product) | 10 |
| Final LQAS Rxn Vol | 30.0 |

*10X oligonucleotide mix = 2 μM each primer and 5 μM each probe and FRET oligonucleotide; see FIGS. 9A-9D for formulations of each 10X triplex oligonucleotide mix named in the table below.
**20X enzyme mix contains 1 unit/μL GoTaq Hot start polymerase (Promega Corp.), 292 ng/μL Cleavase 2.0 FEN-1 flap endonuclease (Hologic, Inc.), 200 mM MOPS, pH 7.5, 150 mM MgCl2, 6.38 mM Tris-HCl, pH 8.0, 15.94 mM KCl, 2 μg/μL BSA, 0.16% Tween-20, 0.16% IGEPAL CA-630, 25% Glycerol.

Reactions were assembled to include primer pairs for the markers in the following triplex combinations:

Triplex reactions

| Assay | A5-FAM | A1-HEX | A3-Q670 |
|---|---|---|---|
| Name | FRET Cassette | FRET Cassette | FRET Cassette |
| ZPB | ZNF781 | PPP2R5C | B3GALT6 |
| CZB | CHST2 | ZNF671 | B3GALT6 |
| TSB | TSPYL5 | SIM2 | B3GALT6 |
| NAE | NDRG4 | ARHGEF4 | EMX1 |
| GQP | GPRIN1 | QKI | PRKCB |
| HSC | HOXA9 | SFMBT2 | CDO1 |
| CFF | CAPN2 | FAIM2 | FAM59B |

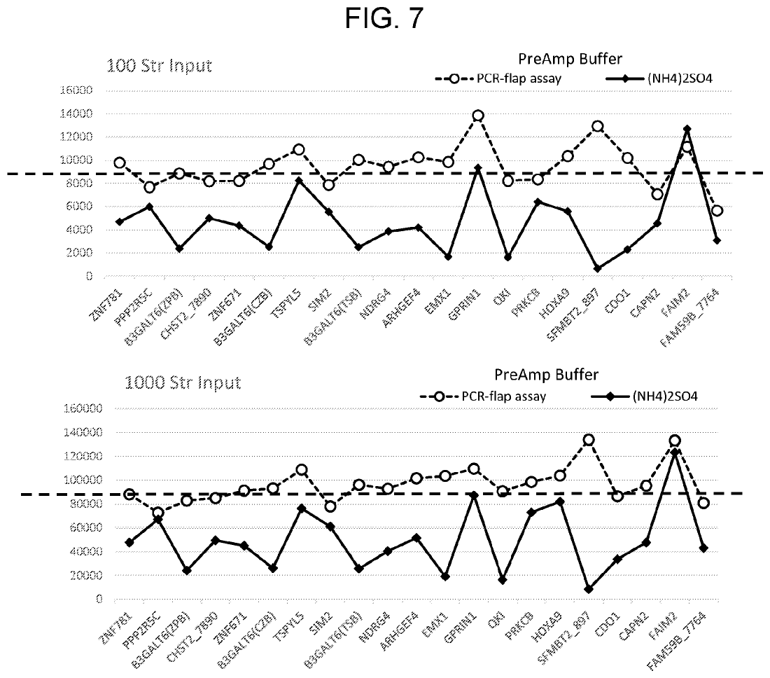

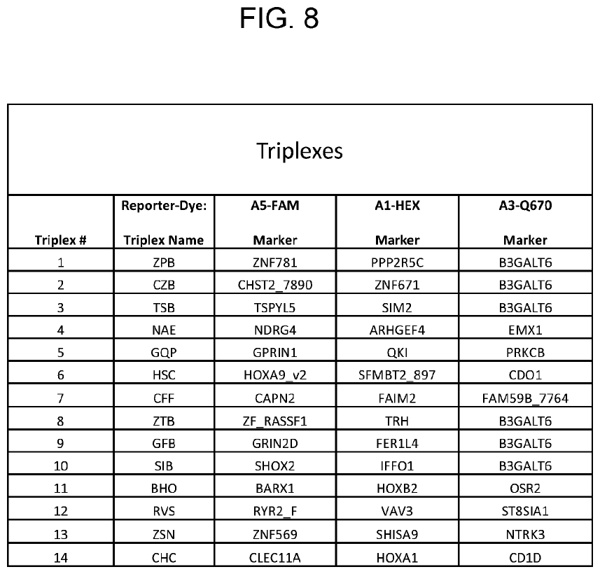

Each reaction was performed in duplicate. Results are shown in FIG. 7, with calculated theoretical yield (81920 strands for the 1000 strand input reactions; 8190 strands for the 100 strand input reactions) being indicated by the horizontal straight dashed line. The amount of deviation above or below the dashed line indicates the signal variation seen for each marker in the two different preamplification buffers. These data show that, although the (NH₄)₂SO₄ PCR buffer includes 6.7 mM MgCl₂, which is relatively high for a PCR buffer, the final results do not match results achieved when the low-bias PCR-flap assay buffer having 7.5 mM MgCl₂ is used in the preamplification reaction. The final PCR-flap assay results after preamplification with the low-bias buffer showed much more consistency of signal (i.e., reduced bias) across all 18 markers, and greater signal overall, as compared to the results measured from the same markers preamplified together in the (NH₄)₂SO₄ PCR buffer.

These data show that the average coefficient of variance across the set of markers tested was reduced from 59% when the (NH₄)₂SO₄ PCR buffer was used to 18% when low-bias PCR-flap assay buffer was used in the preamplification reactions. Amplification efficiency was increased from an average of 59% when the (NH₄)₂SO₄ PCR buffer was used to an average of 117% when low-bias PCR-flap assay buffer was used in the preamplification reactions. These data show that use of low bias (high Mg⁺⁺ PCR-flap assay buffer in the multiplex PCR preamplification both reduced amplification bias between the different targets and increased the signal from each target in follow-on LQAS PCR-flap assay reactions as compared to results observed when the (NH₄)₂SO₄ PCR buffer was used in the preamplification.

### Example 4

### Low-Bias Multiplexed Preamplification with Multiplexed PCR-Flap Assays: Multiple Analytes Reporting One Dye (MAD)

The data above show that using the MOPS buffer with high Mg⁺⁺ for highly multiplexed preamplification allows more uniform co-amplification of the different targets without the need to adjust the primer or primer pair concentrations to reduced marker-to-marker bias. Development of this technology has shown that using these low-bias preamplification conditions allows highly multiplexed analysis of markers without the need to resolve the signals from each individual target.

In the examples above, each preamplified target DNA was subsequently measured in a triplex PCR-flap assay in which each marker in the triplex reports to an individual FRET cassette, such that signal from each individual target nucleic acid can be distinguished from the other two targets in each triplex reaction.

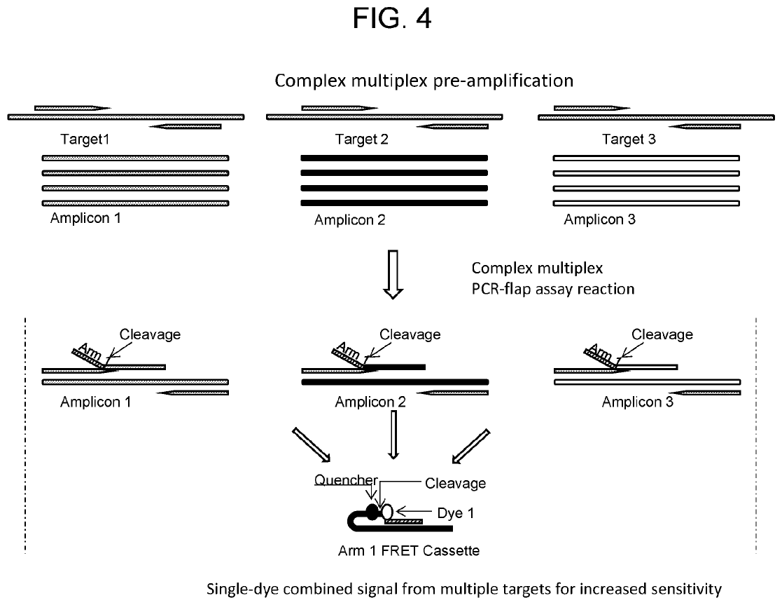

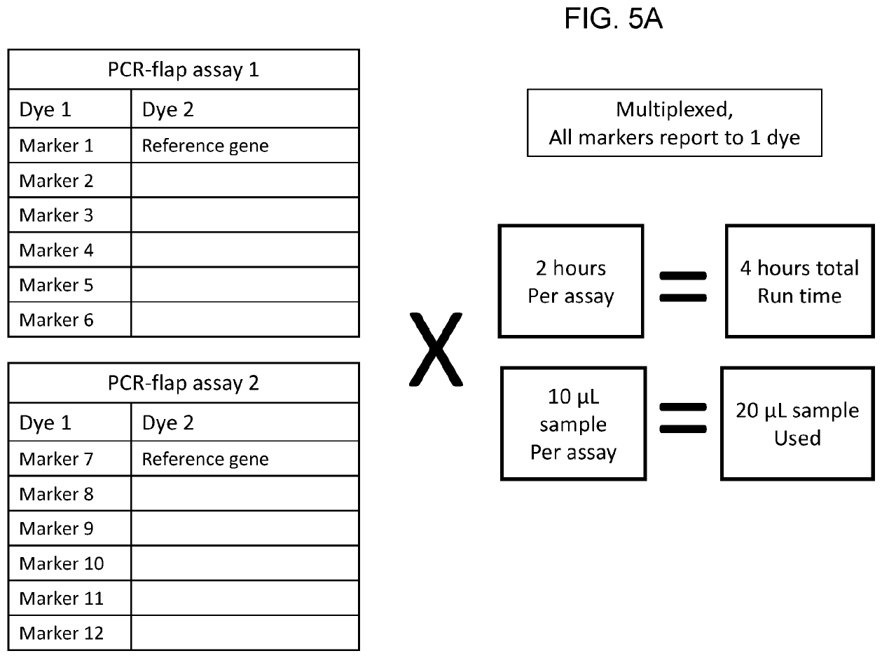

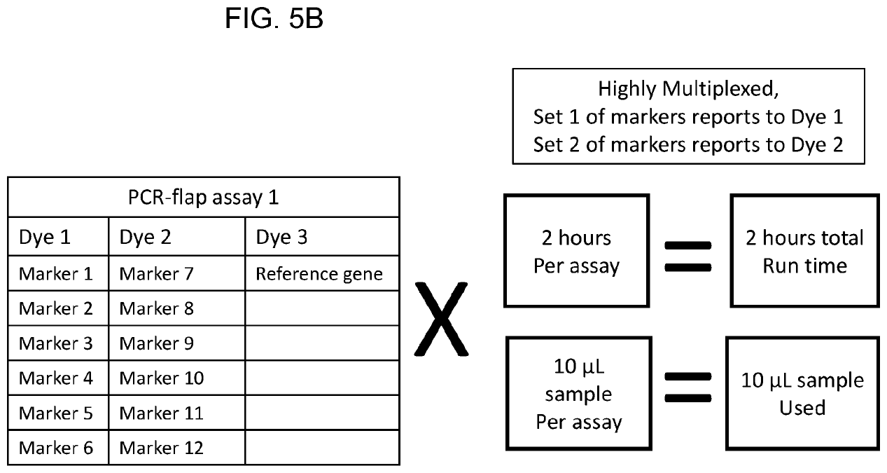

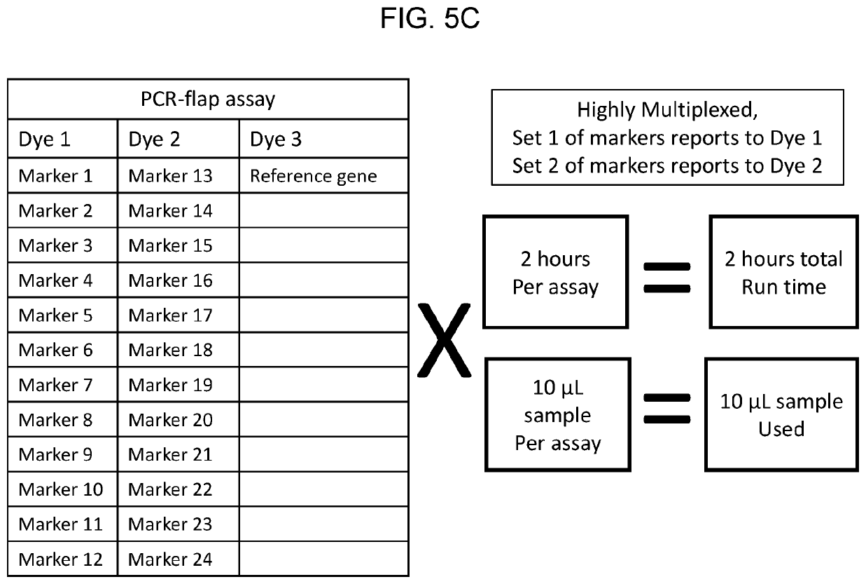

The low-bias preamplification reactions were tested in a configuration in which 3 to 5 different markers in a reaction used the same FRET cassette reporter (Multiple Analytes per Dye, or “MAD” multiplexed reactions), such that the individual signal from any one marker could not be distinguished from each other. Such assay configurations are shown schematically in FIGS. 4 and 5A-5C. The sensitivity and specificity for detecting these markers in cancer samples (DNAs isolated from plasma from subjects known to have cancer) was compared to the results achieved using the triplex configuration, in which the results from different markers are resolved from each other but are combined mathematically to achieve greater sensitivity than is achievable using any single marker.

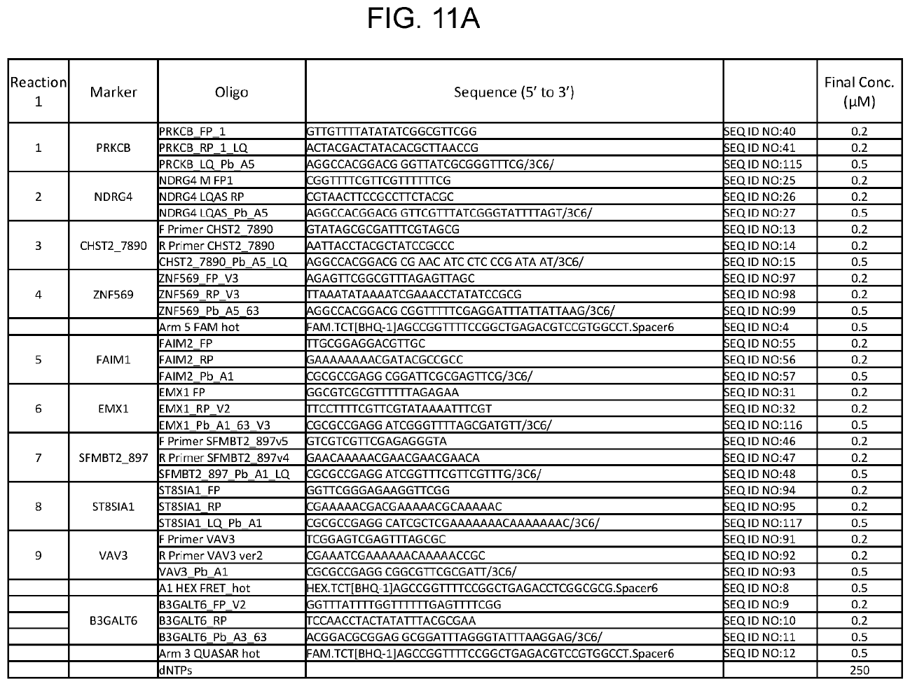

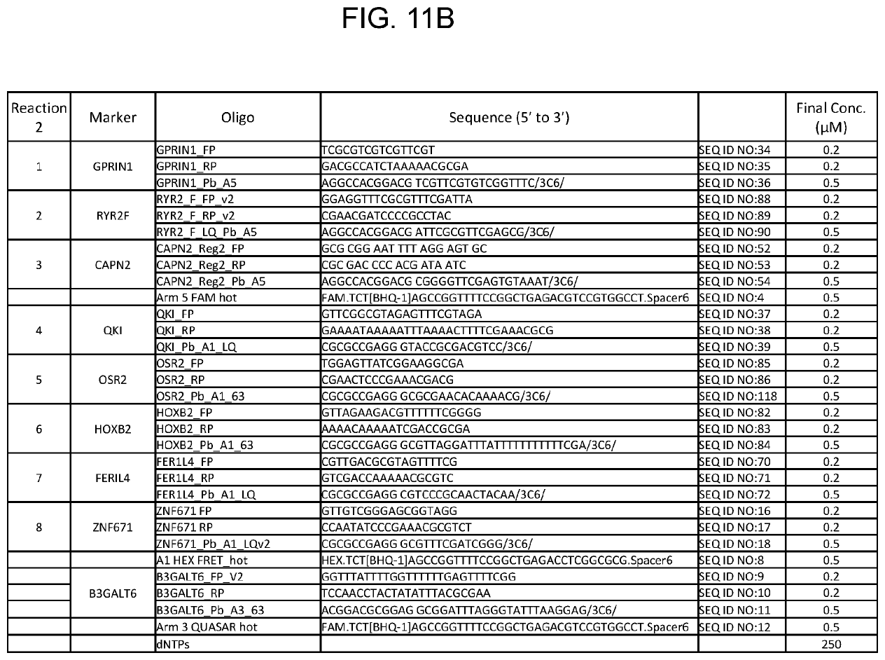

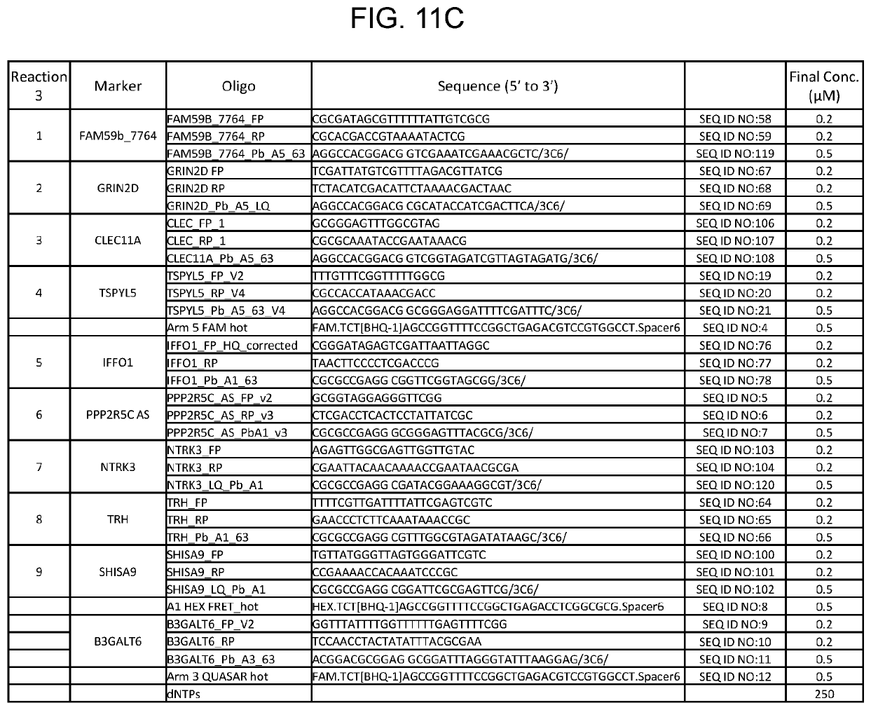

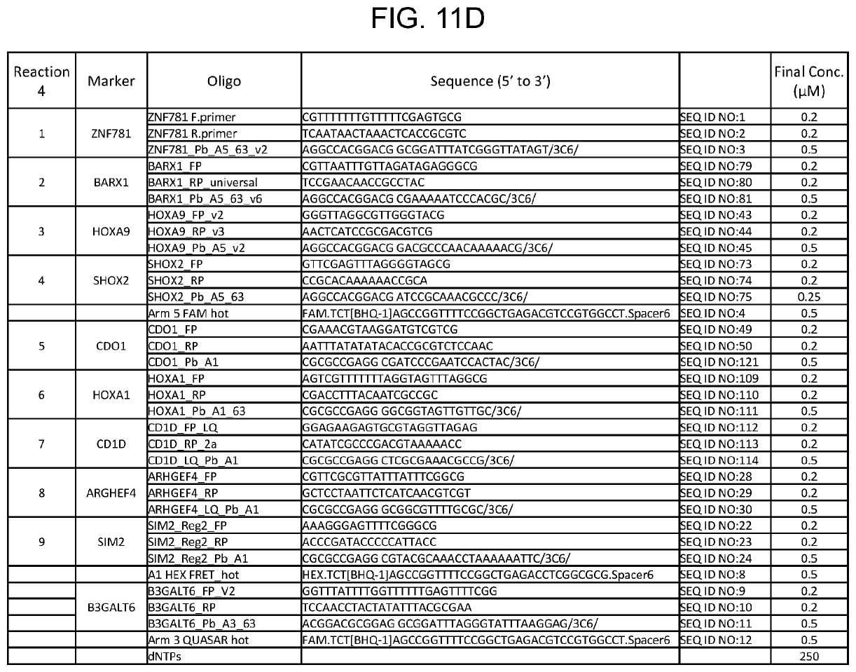

In the MAD multiplexing step, each reaction contained a second FRET cassette for detection of combined signal from a second set of 3-5 markers, and a third FRET cassette to detect reference marker B3GALT6. DNAs from healthy subjects or from subjects having cancer were preamplified in low-bias multiplex conditions described above using the PCR-flap assay buffer described above. The 35 markers plus B3GALT6 reference DNA in the preamplified reaction mixtures were then measured using either the 14 triplex PCR-flap assay reactions using the oligonucleotide combinations shown in FIGS. 9A-9G, or using the 4 MAD multiplexed PCR-flap assays using the oligonucleotide combinations shown in FIGS. 11A-11D.

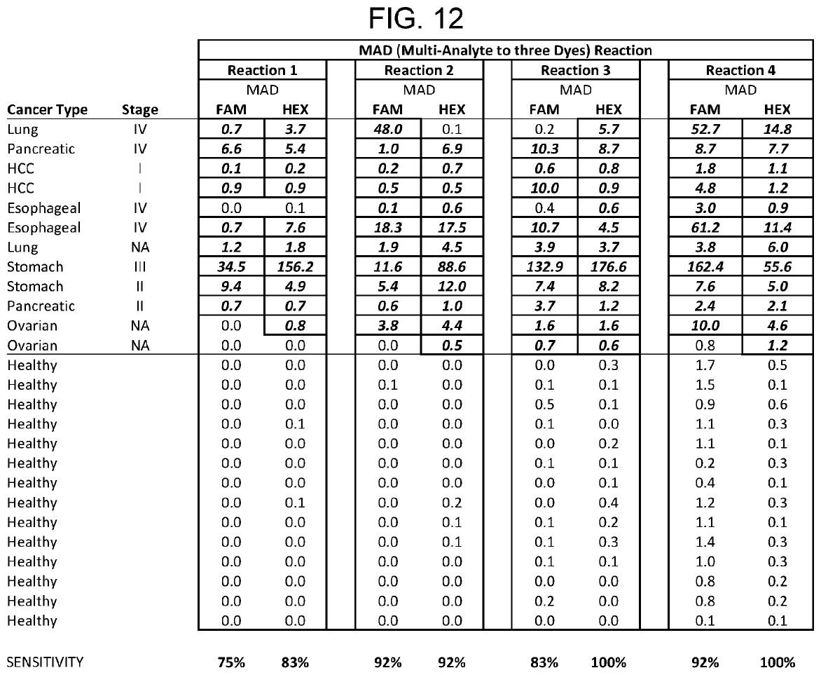

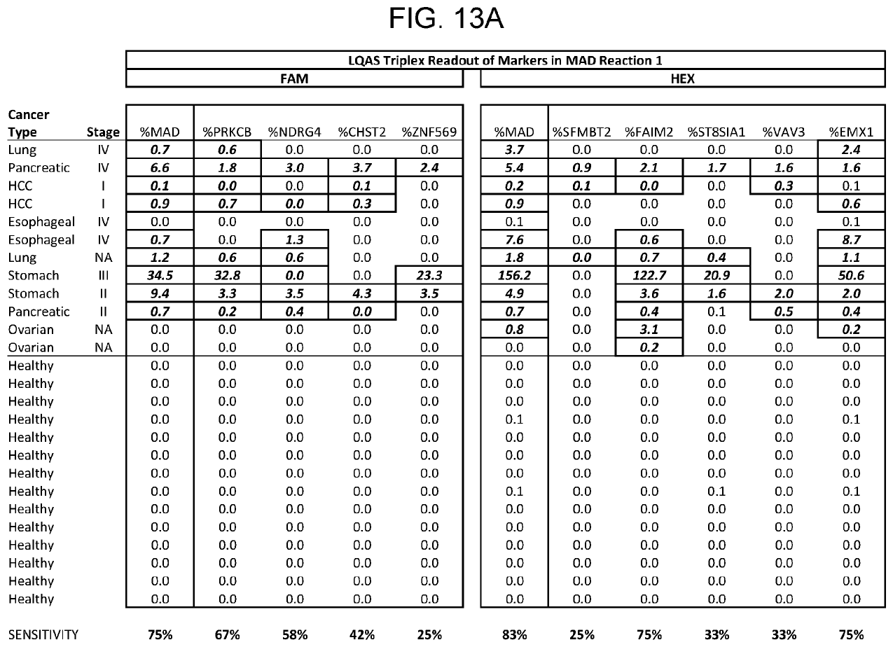

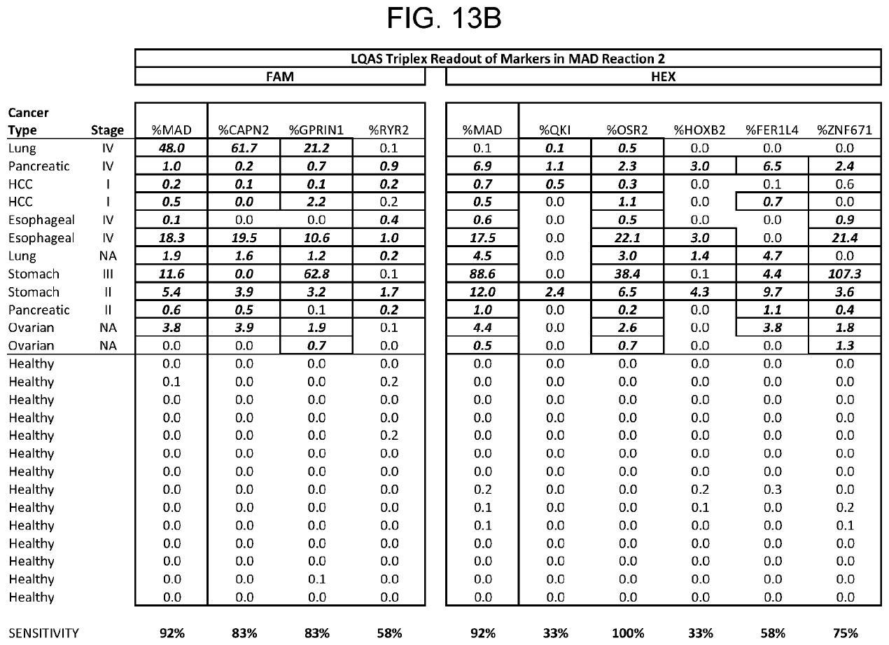

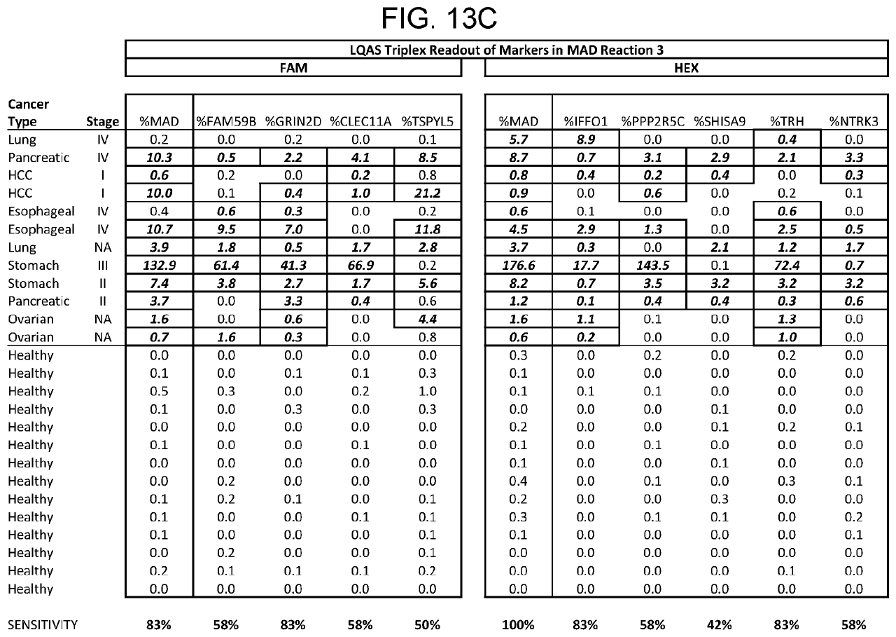

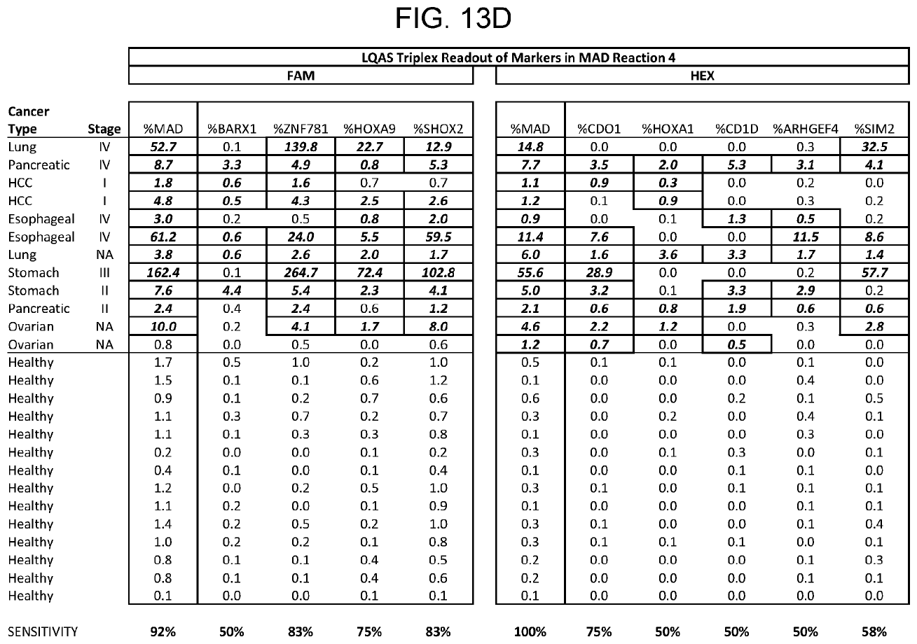

FIG. 12 shows the assay sensitivity results for the four MAD PCR-flap assays covering 35 different methylation marker DNAs and the results for the triplex PCR-flap assays using the same markers is shown in tables in FIGS. 13A-13D. These data show that individual markers measured in the triplex LQAS assays showed maximum % methylation in healthy subjects ranging from 0 to 1.2%, with the maximum % methylation of the markers measured in the MAD groupings in healthy subjects ranging from 0 to 1.7%. The average sensitivity for detecting cancer samples for the individual markers measured using the triplex configuration was 60%, while the average sensitivity for detection of cancer samples using the MAD groupings of markers was 90%.

### Example 5

### Multiplex Analysis Target Nucleic Acids from an Environmental Sample

Environmental samples such as soil and wastewater typically contain diverse populations of viruses and microbes. The present technology finds application in characterizing the microbial and viral populations of such samples.

### Sample Preparation

Nucleic acid is prepared from an environmental sample by standard methods, e.g., using a commercial kit appropriate for extraction of DNA and/or RNA from a particular type of sample. Examples of suitable kits include but are not limited to the MAGMAX Wastewater Ultra Nucleic Acid Isolation kit from ThermoFisher Scientific (MagMAX™ Wastewater Ultra Nucleic Acid Isolation Kit User Guide, 03/2022); RNeasy PowerSoil Total RNA kit from Qiagen (RNeasy PowerSoil Total RNA Kit Handbook 06/2017), and DNeasy PowerSoil Pro kit from Qiagen (DNeasy PowerSoil Pro Kit Handbook 03/2021), the handbooks for which are incorporated by reference herein in their entireties, for all purposes.

### Multiplexed Preamplification of Sample DNA

For preamplification of 30 different target DNAs and a reference DNA, 50 μL of a prepared DNA sample is combined with 200-600 nM each of 31 different primer pairs (62 primers), with the different primers being in essentially equal concentrations, in 75 μL of a preamplification reaction mixture that further comprises 7.5 mM MgCl₂, 10 mM MOPS pH 7.5, 0.3 mM Tris-HCl, pH 8.0, 0.8 mM KCl, 0.1 μg/μL BSA, 0.0001% TWEEN-20 detergent, 0.00010% IGEPAL CA-630 detergent, 250 μM of each dNTP, and 0.025 units/μL HOTSTART GOTAQ DNA polymerase.

The reaction mixture is subjected to thermal cycling as follows:

Pre-Amplification Reaction Thermal Profile:

| Stage | Temp/Time | # Cycles |
|---|---|---|
| Denaturation/Polymerase | 95° C./5′ | 1 |
| activation | | |
| Amplification | 95° C./30″ | 12 |
| | 64° C./1′ | |
| Cooling | 4° C./Hold | 1 |

### Multiplexed Preamplification of Sample RNA

For preamplification of 30 different target RNAs and a reference RNA, 50 μL of an isolated RNA or mixed RNA+DNA sample is combined with 200-600 nM each of 31 different primer pairs (62 primers), with the primers being in essentially equal concentrations, in 75 μL of a pre-amplification reaction mixture that further comprises 0.67 units/μL of MMLV reverse transcriptase, 7.5 mM MgCl₂, 10 mM MOPS pH 7.5, 0.3 mM Tris-HCl, pH 8.0, 0.8 mM KCl, 0.1 μg/μL BSA, 0.0001% TWEEN-20 detergent, 0.0001% IGEPAL CA-630 detergent, 250 μM of each dNTP, and 0.025 units/μL HOTSTART GOTAQ DNA polymerase.

The RT-preamplification reaction mixture is subjected to thermal cycling as follows:

RT-Pre-Amplification Reaction Thermal Profile:

| Stage | Temp/Time | # Cycles |
|---|---|---|
| Reverse Transcription | 42° C./30′ | |
| Denaturation/Polymerase | 95° C./5′ | 1 |
| activation | | |
| Amplification | 95° C./30″ | 12 |
| | 64° C./1′ | |
| Cooling | 4° C./Hold | 1 |

After thermal cycling, the preamplification reaction vessel is centrifuged to collect condensation. A 10 μL aliquot of the preamplification reaction is diluted to 500 μL with 10 mM Tris, 0.1 mM EDTA, optionally comprising bulk fish DNA. Diluted and undiluted preamplified samples may be stored at −20° C.

PCR-Flap Assays from Preamplified Product

### Multiple Different Targets Reporting to Each Dye

Three different PCR-flap assay reactions are performed. Each PCR flap assay reaction mixture comprises primer pairs for 10 different preamplified targets plus one reference or control amplicon. For the 30 different targets plus the reference, three 11-plex flap assay reactions are performed, with the primers, probes, and FRET cassettes combined as follows:

| REACTION 1 | Oligonucleotides | Signal Channel |
|---|---|---|
| Target 1 | Forward primer 1 | FAM |
| | Reverse primer 1 | |
| | Flap probe 1 Arm 5 | |
| Target 2 | Forward primer 2 | |
| | Reverse primer 2 | |
| | Flap probe 2 Arm 5 | |
| Target 3 | Forward primer 3 | |
| | Reverse primer 3 | |
| | Flap probe 3 Arm 5 | |
| Target 4 | Forward primer 4 | |
| | Reverse primer 4 | |
| | Flap probe 4 Arm 5 | |
| Target 5 | Forward primer 5 | |
| | Reverse primer 5 | |
| | Flap probe 5 Arm 5 | |
| | Arm 5 FAM FRET cassette | |
| Target 6 | Forward primer 6 | HEX |
| | Reverse primer 6 | |
| | Flap probe 6 Arm 1 | |
| Target 7 | Forward primer 7 | |
| | Reverse primer 7 | |
| | Flap probe 7 Arm 1 | |
| Target 8 | Forward primer 8 | |
| | Reverse primer 8 | |
| | Flap probe 8 Arm 1 | |
| Target 9 | Forward primer 9 | |
| | Reverse primer 9 | |
| | Flap probe 9 Arm 1 | |
| Target 10 | Forward primer 10 | |
| | Reverse primer 10 | |
| | Flap probe 10 Arm 1 | |
| | Arm 1 HEX FRET cassette | |
| Reference | Forward Primer REF | QUASAR 670 |
| Target | Reverse primer REF | |
| | Flap probe REF Arm 3 | |
| | Arm 3 Q670 FRET cassette | |

Similarly, in Reaction 2, the flap probes for targets 11-15 report to the Arm 5 FAM FRET cassette, the flap probes for targets 16-20 report to the Arm 1 HEX FRET cassette, and the reference target flap probe reports to the Arm 3 Q670 FRET cassette, and in Reaction 3, the flap probes for targets 21-25 report to the Arm 5 FAM FRET cassette, the flap probes for targets 26-30 report to the Arm 1 HEX FRET cassette, and the reference target flap probe reports to the Arm 3 Q670 FRET cassette.

A separate PCR-flap assay master mix is made for each of Reactions 1, 2, and 3, combined as follows:

| | Volume per | For 15 |
|---|---|---|
| Master Mix (per reaction) | reaction (μL) | reactions |
| Water (mol. biol. grade) | 15.50 | 232.5 |
| 10X Oligo Mix 1, 2, or 3* | 3.00 | 45 |
| 20X Enzyme Mix** | 1.50 | 22.5 |
| Total Master mix volume | 20.0 | 300 |

*each 10X oligonucleotide mix = 2 μM each primer, and 5 μM each probe and FRET cassette oligonucleotide.
**20X Enzyme Mix contains 1 unit/μL GOTAQ HOT START DNA polymerase (Promega Corp.), 292 ng/μL Cleavase 2.0 FEN-1 flap endonuclease (Hologic, Inc.), 200 mM MOPS pH 7.5, 150 mM MgCl₂, 6.38 mM Tris-HCl, pH 8.0, 15.95 mM KCl, 2 μg/μL BSA, 0.16% Tween-20 detergent, 0.16% IGEPAL CA-630 detergent, and 25% Glycerol.

For each of the PCR-flap assay reactions 1, 2, and 3, 10 μL of the diluted preamplification reaction product is mixed with 20 μL of the PCR-flap assay master mix, and the reaction mixture is sealed in a reaction vessel (e.g., microtube or well of an assay plate).

The PCR-flap assay reaction mixtures are subjected to thermal cycling as follows, with FAM, HEX, and Q670 fluorescence signal acquired at the indicated points in the amplification cycling:

| | | Ramp | | |
|---|---|---|---|---|
| | | Rate | | FAM/HEX/Q670 |
| Stage | Temp/Time | (° C./sec) | # of Cycles | Acquisition |
| Denaturation/Polymerase | 95° C./3′ | 1.6 | 1 | None |
| activation | | | | |
| Amplification | 92.5° C./10″ | 1.6 | 45 | None |
| | 62.5° C./50″ | 1.6 | | Single |
| Cooling | 40° C./30″ | 1.6 | 1 | None |

In the arrangement for Reaction 1 above, the FAM signal measured during the PCR-flap assay thermal cycling is the total signal from preamplified targets 1-5; the HEX signal is the total signal from preamplified targets 6-10, and the Q670 signal is from the preamplified reference target nucleic acid. Similarly, for Reaction 2, the FAM signal measured during the PCR-flap assay thermal cycling is the total signal from preamplified targets 11-15 and the HEX signal is the total signal from preamplified targets 16-20, and for Reaction 3, the FAM signal measured during the PCR-flap assay thermal cycling is the total signal from preamplified targets 21-25 and the HEX signal is the total signal from preamplified targets 26-30.

### Each Target Reporting to a Different Dye

Signals specific for each of the individual 30 preamplified target DNAs (preamplified from DNA or RNA sample material) are measured in triplex reactions, configured as follows;

| Reaction 1 | Oligonucleotides | Signal Channel |
|---|---|---|
| Target 1 | Forward primer 1 | FAM |
| | Reverse primer 1 | |
| | Flap probe 1 Arm 5 | |
| | Arm 5 FAM FRET cassette | |
| Target 2 | Forward primer 2 | HEX |
| | Reverse primer 2 | |
| | Flap probe 2 Arm 1 | |
| | Arm 1 HEX FRET cassette | |
| Reference | Forward Primer REF | QUASAR 670 |
| Target | Reverse primer REF | |
| | Flap probe REF Arm 3 | |
| | Arm 3 Q670 FRET cassette | |

In a similar arrangement, targets 3-30 are measured pairwise (e.g., 3 and 4, 5 and 6, 7 and 8, etc.), each pair with the reference target, in 14 additional triplex reactions. Results measured for HEX and FAM signals can be normalized to the Q670 signal measured from the reference target so that relative amounts of all markers can be compared across the 15 triplex reactions.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

While certain embodiments of the inventions have been described, these embodiments have been presented by way of example only, and are not intended to limit the scope of the disclosure. Indeed, the novel methods and systems described herein may be embodied in a variety of other forms. Further, various modifications, omissions, substitutions, and variations of the described compositions, methods, systems, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in molecular biology, biochemistry, medical science, or related fields are intended to be within the scope of the following claims. The accompanying claims and their equivalents are intended to cover such forms or modifications as would fall within the scope and spirit of the disclosure. Accordingly, the scope of the present inventions is defined only by reference to the appended claims.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

We claim:

1. A method of analyzing a mixture suspected of comprising multiple target nucleic acids, comprising:
a) treating a sample suspected of comprising multiple different target nucleic acids in a preamplification reaction mixture to produce a multiplex preamplified mixture,
wherein the preamplification reaction mixture comprises at least 4 different target-specific primer pairs for producing amplified regions from at least 4 different target nucleic acids, if present in the sample, and at least one reference primer pair for producing an amplified region from a reference nucleic acid;
wherein each of the primers in the at least 4 different target-specific primer pairs and the at least one reference primer pair are in essentially the same concentrations;
b) adding a portion of the multiplex preamplified mixture to a multiplex PCR assay reaction mixture comprising:
i) additional amounts of each of the at least 4 different target-specific primer pairs and the reference primer pair, wherein the primers in the additional amounts of the at least 4 different target-specific primer pairs and the at least one reference primer pair are added in essentially the same amounts;
ii) at least 4 different target-specific probe oligonucleotides, wherein each target-specific probe oligonucleotide specifically hybridizes to a different one of the amplified regions from the at least 4 different target nucleic acids, if amplified in step a), wherein the at least 4 different target-specific probe oligonucleotides comprise a first label; and
iii) a reference probe flap oligonucleotide that specifically hybridizes to the amplified region from the reference nucleic acid, wherein the reference probe flap oligonucleotide comprises a second label;
and
c) conducting a PCR assay with said multiplex PCR assay reaction mixture, wherein the reference nucleic acid region and each of said at least 4 different target regions, if amplified in step a), are amplified in the PCR assay reaction mixture, wherein target-specific probe oligonucleotides and the reference probe oligonucleotide specifically hybridize to target regions and the reference nucleic acid region amplified in the multiplex PCR assay reaction mixture and are cleaved to release first and second labels, wherein said released first and second labels are measured.

2. The method of claim 1, wherein the preamplification reaction mixture comprises a low-bias amplification buffer.

3. The method of claim 2, wherein the multiplex PCR assay reaction mixture comprises a low-bias amplification buffer, preferably the same low-bias amplification buffer used in the preamplification reaction mixture.

4. The method of any one of claims 1-3, wherein in step b) the at least four different target-specific probe oligonucleotides and the reference probe flap oligonucleotide are present in said multiplex PCR assay reaction mixture in essentially the same concentrations.

5. The method of any one of claims 1-4, wherein said first label comprises a first 5′ flap sequence, wherein the first 5′ flap sequence is not substantially complementary to any of the amplified regions from the at least 4 different target nucleic acids.

6. The method of claim 5, wherein said second label comprises a second 5′ flap sequence, wherein the second 5′ flap sequence is different than the first 5′ flap sequence and is not substantially complementary to the amplified region from the reference nucleic acid.

7. The method of claim 5 or claim 6, wherein the PCR assay reaction mixture further comprises a first FRET cassette labeled with a first fluorophore, the first FRET cassette comprising a sequence complementary to the first 5′ flap sequence, and/or a second FRET cassette labeled with a second fluorophore, the second FRET cassette comprising a sequence complementary to the second 5′ flap sequence.

8. The method of any one of claims 1-7, wherein the PCR assay reaction mixture further comprises a flap endonuclease, preferably a FEN-1 endonuclease, preferably an archaeal FEN-1 endonuclease.

9. The method of any one of claims 1-8, wherein said first label comprises a first FRET system comprising a first fluorophore, and wherein said second label comprises a second FRET system comprising a second fluorophore, and wherein fluorescence from the first fluorophore and the second fluorphore is measured during said PCR assay.

10. The method of any one of claims 1-9, wherein the at least 4 different target-specific primer pairs comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different target-specific primer pairs.

11. The method of any one of claims 1-10, wherein the at least 4 different target-specific probe oligonucleotides comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different target-specific probe oligonucleotides.

12. The method of any one of claims 1-11, wherein the concentrations of the at least four different target-specific primer pairs and the reference primer pair in the PCR assay reaction mixture are essentially the same as the concentrations of the at least four different target-specific primer pairs and the reference primer pair in the preamplification reaction mixture.

13. The method of any one of claims 1-12, wherein the low-bias amplification buffer comprises 3-(n-morpholino) propanesulfonic acid (MOPS) buffer and at least about 6 mM, preferably 6.1, 6.2, 6.5, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, or 11.0 mM Mg⁺⁺.

14. The method of claim 13, wherein the low-bias amplification buffer comprises about 7.5 mM Mg⁺⁺.

15. The method of any one of claims 1-14, wherein the preamplification reaction mixture comprises at least one additional target-specific primer pair for producing an amplified region from an additional target nucleic acid, if present in the sample, that is different from the at least four different target nucleic acids and from the reference nucleic acid, and wherein the multiplex PCR assay reaction mixture further comprises:
i) an additional amount of at the at least 1 additional target-specific primer pair in essentially the same amount as the additional amounts of the at least four different target-specific primer pairs; and
ii) at least 1 additional target-specific probe oligonucleotide that specifically hybridizes to an amplified region from the at least 1 additional target nucleic acid, if amplified in step a), the at least 1 additional target-specific probe oligonucleotide having a third label, wherein the third label is different than the first and the second label.

16. The method of claim 15, wherein said third label comprises a third 5′ flap sequence, wherein the third 5′ flap sequence is different than the first 5′ flap sequence and the second 5′ flap sequence and is not substantially complementary to the amplified region from the additional target nucleic acid.

17. The method of claim 16, wherein the PCR assay reaction mixture further comprises a third FRET cassette labeled with a third fluorophore, the third FRET cassette comprising a sequence complementary to the third 5′ flap sequence.

18. The method of any one of claims 14-17, wherein the at least 1 additional target-specific primer pair comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different additional target-specific primer pairs for producing amplified regions from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different additional target nucleic acids, if present in the sample; and
wherein the multiplex PCR assay reaction mixture further comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 additional target-specific probe oligonucleotides comprising the third label, wherein the additional target-specific probe oligonucleotides specifically hybridize to amplified regions from the 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different additional target nucleic acids, if amplified in step a).

19. A method of analyzing a sample for multiple target nucleic acids in a PCR-flap assay, comprising:
a) treating nucleic acid sample in a preamplification reaction mixture, to produce a multiplex preamplified mixture,
wherein the preamplification reaction mixture comprises at least 4 different target-specific primer pairs for producing amplified regions from at least 4 different target nucleic acids, if present in the sample, and at least one reference primer pair for producing an amplified region from a reference nucleic acid;
wherein each of the primers in the at least 4 different target-specific primer pairs and the at least one reference primer pair are in essentially the same concentrations;
b) adding a portion of the multiplex preamplified mixture to a multiplex PCR-flap assay reaction mixture comprising:
i) additional amounts of each of the at least 4 different target-specific primer pairs and the reference primer pair, wherein the primers in the additional amounts of the at least 4 different target-specific primer pairs and the at least one reference primer pair are added in essentially the same amounts;
ii) at least 4 different target-specific flap probe oligonucleotides, wherein each target-specific flap probe oligonucleotide specifically hybridizes to a different one of the amplified regions from the at least 4 different target nucleic acids, if amplified in step a),
wherein each one of the at least 4 different target-specific flap probe oligonucleotides comprises a first 5′ flap sequence, wherein the first 5′ flap sequence is not substantially complementary to any of the amplified regions from the at least 4 different target nucleic acids;
iii) a reference flap probe oligonucleotide that specifically hybridizes to the amplified region from the reference nucleic acid, the reference flap probe oligonucleotide having a second 5′ flap sequence, wherein the second 5′ flap sequence is different than the first 5′ flap sequence and is not substantially complementary to the amplified region from the reference nucleic acid;
iv) a first FRET cassette labeled with a first fluorophore, the first FRET cassette comprising a sequence complementary to the first 5′ flap sequence;
v) a second FRET cassette labeled with a second fluorophore, the second FRET cassette comprising a sequence complementary to the second 5′ flap sequence; and
vi) a PCR-flap assay buffer;
and
c) conducting a PCR-flap assay with said multiplex PCR-flap assay reaction mixture, wherein the reference nucleic acid and each of said at least 4 different target regions, if amplified in step a), are amplified in the PCR-flap assay reaction mixture, and fluorescence from the first fluorophore and the second fluorphore are measured.

20. The method of claim 19, wherein the preamplification reaction mixture comprises a low-bias amplification buffer.

21. The method of claim 19 or claim 20, wherein in step b) the at least 4 different target-specific flap probe oligonucleotides and the reference flap probe oligonucleotide present in said multiplex PCR-flap assay reaction mixture are in essentially the same concentrations.

22. The method of any one of claims claim 1-21, wherein treating the nucleic acid in the preamplification reaction mixture comprises thermal cycling the preamplification reaction mixture for fewer than 20 thermal cycles, preferably fewer than 19, 18, 17, 16, 15, 14, 13, 12, 11, 10, 9, 8, 7, 6, or 5 thermal cycles.

23. The method of any one of claims 19-21, wherein the concentrations of the at least four different target-specific primer pairs and the reference primer pair in the PCR-flap assay reaction mixture are essentially the same as the concentrations of the at least four different target-specific primer pairs and the reference primer pair in the preamplification reaction mixture.

24. The method of any one of claims 1-23, further comprising diluting at least a portion of the multiplex preamplified mixture prior to step b).

25. The method of any one of claims 19-24, wherein the preamplification reaction mixture comprises at least one additional target-specific primer pair for producing an amplified region from an additional target nucleic acid, if present in the sample, that is different from the at least four different target nucleic acids and from the reference nucleic acid, and wherein the multiplex PCR-flap assay reaction mixture further comprises:
i) an additional amount of the at least 1 additional target-specific primer pair in essentially the same amount as the additional amounts of the at least four different target-specific primer pairs;
ii) at least 1 additional target-specific flap probe oligonucleotide that specifically hybridizes to an amplified region from the at least 1 additional target nucleic acid, if amplified in step a), the at least 1 additional target-specific flap probe oligonucleotide having a third 5′ flap sequence, wherein the third 5′ flap sequence is different than the first and the second 5′ flap sequences and is not substantially complementary to the amplified region from the at least 1 additional target nucleic acid; and
iii) a third FRET cassette labeled with a third fluorophore, the third FRET cassette comprising a sequence complementary to the third 5′ flap sequence.

26. The method of any one of claims 19-25, wherein the at least 1 additional target-specific primer pair comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different additional target-specific primer pairs for producing amplified regions from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different additional target nucleic acids, if present in the sample; and
wherein the multiplex PCR-flap assay reaction mixture further comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 additional target-specific flap probe oligonucleotides comprising the third label, wherein the additional target-specific flap probe oligonucleotides specifically hybridize to amplified regions from the 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different additional target nucleic acids, if amplified in step a).

27. The method of any one of claims 1-26, wherein the sample suspected of comprising multiple different target nucleic acids is prepared from a sample comprising one or more of soil, water, plant material, an animal or human sample comprising one or more of stool, tissue, sputum, mucus, blood or a blood product selected from plasma, serum, whole blood, an organ excretion, and urine.

28. The method of claim 27, wherein the sample suspected of comprising multiple different target nucleic acids comprises cell-free DNA isolated from plasma.

29. The method of claim 28, wherein the sample suspected of comprising multiple different target nucleic acids comprises cDNA prepared from RNA isolated from plasma.

30. The method of claim 28 or claim 29, wherein the preamplification reaction mixture of step a) has a total volume, wherein the sample suspected of comprising multiple different target nucleic acids is prepared from at least one mL of plasma, and is at least 20 to 50% of the total volume of the preamplification reaction mixture of step a).

31. A method of analyzing a sample for at least 10 different target nucleic acids in a single PCR-flap assay reaction, the method comprising:
a) treating nucleic acid comprising multiple different target nucleic acids in a preamplification reaction mixture comprising a PCR-flap assay buffer, to produce a multiplex preamplified mixture,
wherein the preamplification reaction mixture comprises at least 10 different target-specific primer pairs for producing amplified regions from at least 10 different target nucleic acids in the sample and at least one reference primer pair for producing an amplified region from a reference nucleic acid, wherein each of the primers in the at least 10 different target-specific primer pairs and the at least one reference primer pair are in essentially the same concentration in the preamplification reaction mixture;
b) adding a portion of the multiplex preamplified mixture to a multiplex PCR-flap assay reaction mixture comprising:
i) additional amounts of each of the at least 10 different target-specific primer pairs and the reference primer pair, wherein the primers in the additional amounts of the at least 10 different target-specific primer pairs and the at least one reference primer pair are in essentially the same concentration in the multiplex PCR-flap assay reaction mixture;
ii) at least 10 different target-specific flap oligonucleotides, wherein each target-specific flap oligonucleotide specifically hybridizes to a different one of the amplified regions from the at least 10 different target nucleic acids,
wherein the at least 10 different target-specific flap oligonucleotides are divided into a first group and a second group, wherein each one of the flap oligonucleotides in the first group comprises a first 5′ flap sequence, and wherein each one of the flap oligonucleotides in the second group comprises a second 5′ flap sequence;
iii) a reference flap oligonucleotide that specifically hybridizes to the amplified region from the reference nucleic acid, the reference flap oligonucleotide having a third 5′ flap sequence, wherein the third 5′ flap sequence is different than the first 5′ flap sequence and the second 5′ flap sequence;
iv) a first FRET cassette labeled with a first fluorophore, the first FRET cassette comprising a sequence complementary to the first 5′ flap sequence;
v) a second FRET cassette labeled with a second fluorophore, the second FRET cassette comprising a sequence complementary to the second 5′ flap sequence;
vi) a third FRET cassette labeled with a third fluorophore, the third FRET cassette comprising a sequence complementary to the third 5′ flap sequence; and
vi) PCR-flap assay buffer;
and
c) conducting a PCR-flap assay with said multiplex PCR-flap assay reaction mixture, wherein the reference nucleic acid and each of said at least 10 different target regions are amplified in the PCR-flap assay reaction mixture, and fluorescence from the first fluorophore, the second fluorphore, and the third fluorphore are measured.

32. The method of claim 31, wherein conducting the PCR-flap assay with said multiplex PCR-flap assay reaction mixture comprises thermal cycling the multiplex PCR-flap assay reaction mixture for at least 25 thermal cycles, preferably more than 30, 31, 32, 33, 34, 35, 36, 37, 38, 39, 40, 41, 42, 43, 44, or 45 thermal cycles.

33. The method of claim 31 or claim 32, wherein fluorescence from the first fluorophore, the second fluorphore, and the third fluorphore is measured during thermal cycling.

34. A composition comprising in a mixture:
a) a group of oligonucleotides comprising:
i) a first set of at least four different target-specific primer pairs for producing amplified regions from a first group of at least four different target nucleic acids;
ii) at least one reference primer pair for producing an amplified region from a reference nucleic acid;
wherein each of the primers in the at least four different target-specific primer pairs and the at least one reference primer pair are in essentially the same concentration;
iii) a first set of at least four different target-specific flap oligonucleotides, wherein each target-specific flap oligonucleotide specifically hybridizes to a different one of the amplified regions from the group of at least four different target nucleic acids,
wherein each one of the at least four different flap oligonucleotides comprises a first 5′ flap sequence;
iv) a reference flap oligonucleotide that specifically hybridizes to the amplified region from the reference nucleic acid, the reference flap oligonucleotide having a second 5′ flap sequence,
wherein the second 5′ flap sequence is different than the first 5′ flap sequence;
v) a first FRET cassette labeled with a first fluorophore, the first FRET cassette comprising a sequence complementary to the first 5′ flap sequence and not substantially complementary to the second 5′ flap sequence; and
vi) a second FRET cassette labeled with a second fluorophore, the second FRET cassette comprising a sequence complementary to the second 5′ flap sequence and not substantially complementary to the first 5′ flap sequence; and
b) dNTPs.

35. The composition of claim 34, wherein the first set of at least four different target-specific flap oligonucleotides comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different flap oligonucleotides.

36. The composition of claim 34 or claim 35, wherein the first set of at least four different target-specific primer pairs comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different primer pairs.

37. The composition of any one of claims 34-36, further comprising:
vii) a second set of different target-specific primer pairs for producing amplified regions from a second group of different target nucleic acids;
wherein each of the primers in the second set of different target-specific primer pairs are in essentially the same amount or concentration as the amount or concentration of the primers in the first set of target-specific primer pairs;
viii) a second set of different target-specific flap oligonucleotides, wherein each target-specific flap oligonucleotide specifically hybridizes to a different one of the amplified regions from the second group of different target nucleic acids,
wherein each one of the flap oligonucleotides in the second set of target-specific flap oligonucleotides comprises a third 5′ flap sequence; and
ix) a third FRET cassette labeled with a third fluorophore, the third FRET cassette comprising a sequence complementary to the third 5′ flap sequence and not substantially complementary to the first 5′ flap sequence or the second 5′ flap sequence.

38. The composition of claim 37, wherein the second set of different target-specific flap oligonucleotides comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different flap oligonucleotides.

39. The composition of claim 37 or claim 38, wherein the second set of different target-specific primer pairs comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different primer pairs.

40. The composition of any one of claims 37-39, further comprising one or more of:
x) a DNA polymerase, preferably a thermostable DNA polymerase;
xi) a flap endonuclease, preferably a FEN-1 endonuclease, preferably an archaeal FEN-1 endonuclease; and
xii) a low-bias amplification buffer.

41. The composition of any one of claims 37-40, further comprising:
xiii) a portion of a multiplex preamplified mixture amplified in a low-bias amplification buffer using at least four different target-specific primer pairs and at least one reference primer pair in essentially the same concentrations, the multiplex preamplified mixture comprising amplified regions of the first group of at least four different target nucleic acids and the reference nucleic acid.

42. A kit comprising:
a) a mixture comprising a group of oligonucleotides comprising:
i) a first set of at least four different target-specific primer pairs for producing amplified regions from a first group of at least four different target nucleic acids in the sample;
ii) at least one reference primer pair for producing an amplified region from a reference nucleic acid in the sample;
wherein each of the primers in the at least four different target-specific primer pairs and the at least one reference primer pair are in essentially the same amount or concentration;
iii) a first set of at least four different target-specific flap oligonucleotides, wherein each target-specific flap oligonucleotide specifically hybridizes to a different one of the amplified regions from the group of at least four different target nucleic acids,
wherein each one of the at least four different flap oligonucleotides comprises a first 5′ flap sequence;
iv) a reference flap oligonucleotide that specifically hybridizes to the amplified region from the reference nucleic acid, the reference flap oligonucleotide having a second 5′ flap sequence,
wherein the second 5′ flap sequence is different than the first 5′ flap sequence;
v) a first FRET cassette labeled with a first fluorophore, the first FRET cassette comprising a sequence complementary to the first 5′ flap sequence and not substantially complementary to the second 5′ flap sequence; and
vi) a second FRET cassette labeled with a second fluorophore, the second FRET cassette comprising a sequence complementary to the second 5′ flap sequence and not substantially complementary to the first 5′ flap sequence.

43. The kit of claim 42, wherein the first set of at least four different target-specific flap oligonucleotides comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different flap oligonucleotides.

44. The kit of claim 42 or claim 43, wherein the first set of at least four different target-specific primer pairs comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different primer pairs.

45. The kit of any one of claims 42-44, wherein the mixture further comprising:
vii) a second set of different target-specific primer pairs for producing amplified regions from a second group of different target nucleic acids in a sample;
wherein each of the primers in the second set of different target-specific primer pairs are in essentially the same amount or concentration as the amount or concentration of the primers in the first set of target-specific primer pairs;
viii) a second set of different target-specific flap oligonucleotides, wherein each target-specific flap oligonucleotide specifically hybridizes to a different one of the amplified regions from the second group of different target nucleic acids,
wherein each one of the flap oligonucleotides in the second set of target-specific flap oligonucleotides comprises a third 5′ flap sequence; and
ix) a third FRET cassette labeled with a third fluorophore, the third FRET cassette comprising a sequence complementary to the third 5′ flap sequence and not substantially complementary to the first 5′ flap sequence or the second 5′ flap sequence.

46. The kit of claim 45, wherein the second set of different target-specific flap oligonucleotides comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different flap oligonucleotides.

47. The kit of claim 45 or claim 46, wherein the second set of different target-specific primer pairs comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different primer pairs.

48. The kit of any one of claims 42-47, further comprising one or more of:
b) a DNA polymerase, preferably a thermostable DNA polymerase;
c) a flap endonuclease, preferably a FEN-1 endonuclease, preferably an archaeal FEN-1 endonuclease;
d) a low-bias amplification buffer; and
e) dNTPs.

49. The kit of any one of claims 42-48, further comprising:
f) in a second mixture, portions of the group of oligonucleotides comprising:
i) the first set of at least four different target-specific primer pairs for producing amplified regions from a first group of at least four different target nucleic acids in a sample;
ii) the at least one reference primer pair for producing an amplified region from a reference nucleic acid in the sample;
wherein each of the primers in the at least four different target-specific primer pairs and the at least one reference primer pair are in essentially the same amount or concentration.

50. The kit of any one of claims 42-49, wherein the mixture of a) is in dried form or in the form of a solution.

51. The kit of claim 49 or 50, wherein the mixture of f) is in dried form or in the form of a solution.

52. The kit of any one of claims 48-51, wherein the low-bias amplification buffer comprises 3-(n-morpholino) propanesulfonic acid (MOPS) buffer and a concentration of Mg⁺⁺ to provide a final concentration of Mg⁺⁺ in a PCR reaction mixture of at least about 6 mM, preferably 6.1, 6.2, 6.5, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, or 11.0 mM Mg⁺⁺.

53. The method of claim 52, wherein the low-bias amplification buffer comprises a concentration of Mg⁺⁺ to provide a final concentration of Mg⁺⁺ in a PCR reaction mixture of about 7.5 mM Mg^{++.}
